(19) United States
(12) Patent Application Publication
(10) Pub. No.: US 2010/0172927 A1
(43) Pub. Date: Jul. 8, 2010

(54) Vaccines Against Chlamydial Infection

(76) Inventors: Mark Alderson, Bainbridge, WA (US); Rhea Coler, Seattle, WA (US); Yves Lobet, Rixensart (BE); Jean-Francois L. Maisonneuve, Federal Way, WA (US); Pascal Mettens, Rixensart (BE); Peter Probst, Seattle, WA (US); Steven Reed, Seattle, WA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/442,538

(22) PCT Filed: Oct. 3, 2007
(86) PCT No.: PCT/EP2007/060500

§ 371 Date: Jan. 22, 2010

Related U.S. Application Data
(60) Provisional application No. 60/828,092, filed on Oct. 4, 2006.

(51) Int. Cl. A61K 39/118; A61P 31/04; A61P 27/00; C07K 14/295

(52) U.S. Cl. 424/190.1; 530/350

(57) ABSTRACT

Methods and compositions for the treatment or prevention of ocular Chlamydia trachomatis infection. Compositions comprise one or more Chlamydia trachomatis proteins, immunogenic fragments thereof or polynucleotides encoding such proteins or fragments.

### FIELD OF THE INVENTION

The present invention relates generally to the treatment or prevention of Chlamydial infection. In particular, the invention relates to a method for the treatment or prevention of ocular Chlamydia trachomatis infection and related aspects.

### BACKGROUND OF THE INVENTION

Chlamydiae are intracellular bacterial pathogens that are responsible for a wide variety of important human and animal infections.

Chlamydia trachomatis is transmitted between human beings through social or sexual contact. A number of Chlamydia trachomatis serovars exist, and although the identification and classification of serovars continues to evolve, at least 18 have been reported to date. Serovars A to C are primarily associated with ocular trachoma, serovars D to K with oculogenital disease and serovars L1 to L3 with lymphogranuloma venereum (LGV) (Brunham, R C et al. J. Nat. Rev. Immunol. 2005 5:149-161). However, such disease associations are not absolute, for example, serovar B has been found in genital tract isolates (Caldwell, H B et al. J. Clin. Invest. 2003 111(11):1757-1769).

Chlamydia trachomatis is one of the most common causes of sexually transmitted diseases and can lead to pelvic inflammatory disease (PID), resulting in tubal obstruction and infertility. Chlamydia trachomatis may also play a role in male infertility. In 1990, the cost of treating PID in the US was estimated to be $4 billion. The World Health Organisation estimated that in 1999 over 90 million new cases of sexually transmitted Chlamydia trachomatis occurred worldwide (Global Prevalence and Incidence of Selected Curable Sexually Transmitted Infections, World Health Organisation, Geneva, 2001). Furthermore, ulcerative sexually transmitted diseases such as Chlamydia trachomatis infection are a major risk factor for HIV acquisition (Brunham, R C et al. J. Nat. Rev. Immunol. 2005 5:149-161; Igietseme, J U et al. Expert Rev. Vaccines 2003 2(1):129-146).

Often Chlamydia trachomatis infection is asymptomatic and subclinical, such that severe and often irreversible complications may present as the first symptoms of genital infection. Infants born from a mother with a genital Chlamydia trachomatis infection may develop pneumonia and Chlamydia trachomatis is considered the most common causative agent of pneumonia during the first six months of life (de la Maza, L M et al. Curr. Opin. Investig. Drugs 2002 3(7):980-986). Trachoma, due to ocular infection with Chlamydia trachomatis, is the leading cause of preventable blindness worldwide and is estimated to affect 300-500 million people (West, S K Prog. Ret. Eye Res. 2004 23:381-401). Current treatment involves the use of antibiotics such as tetracycline (daily, for a period of 4 to 6 weeks) or azithromycin (single dose). Although effective in combating infection, re-infection generally occurs due to the endemic nature of the infection. Repeated infection over many years leads to scarring of the eyelid, distortion of the lid margin and rubbing of the eye lashes against the cornea (trichiasis). Constant trauma to the cornea is both painful and leads to corneal opacity and blindness (Mabey, D C W et al. The Lancet 2003 362:223-229).

Individuals who have been exposed to Chlamydia trachomatis have been shown to develop some degree of natural immunity to re-infection, at least in the case of the same serovar (Katz, B P et al. Sex. Transm. Dis. 1987 14:160-164), although the extent of protection may depend upon the time elapsed since the prior infection occurred. Age has also been shown to be important in the duration of infection, with older individuals demonstrating a shorter duration of infection by ocular Chlamydia trachomatis (Bailey, R et al. Epidemiol. Infect. 1999 123:479-486), again suggesting the existence of adaptive immunological protection. It has been suggested that the use of antibiotics may in fact hamper the development of natural immunity to Chlamydia trachomatis (Brunham, R C et al. J. Nat. Rev. Immunol. 2005 5:149-161; Atik, B et al. J.A.M.A. 2006 296(12): 1488-1497).

Chlamydia trachomatis infection thus constitutes a significant health problem both in developed and developing countries. In light of the public health concerns, and the fact that the cost of current treatments is excessive in many developing countries, the development of vaccines for Chlamydia species has been an important research target. As the genomic make-up of Chlamydia trachomatis is relatively stable, and since the presence of animal reservoirs is negligible, even vaccines with limited efficacy may have a significant impact on the prevalence of infections.

The major outer membrane protein (Momp) constitutes approximately 60% of the protein mass of the bacterial outer membrane and is believed to be important in the determination of serotype specificity. The amino acid sequence contains four regions which are externally exposed and in which the majority of sequence variations occur. Of the ca. 400 amino acids in the Momp sequence, up to 70 amino acids differ between Momp from different serovars. Particularly surprising is the finding that serovar grouping based on amino acid sequence identity does not correspond to the serovar grouping based on the associated disease states (i.e. ocular, oculogenital and LGV) (Stothard, D R et al. Infect. Immun. 1998 66(8):3618-3625). Similarly, nucleotide sequence identity comparisons for the ompA gene which encodes Momp do not correspond to disease states (Meijer, A et al. J. Bateriol. 1999 181(15):4469-4475; Lysen, M et al. J. Clin. Microbiol. 2004 42(4):1641-1647). Monoclonal antibodies for Momp are effective in culture and in some animal models, however, protection can be limited and is generally serovar specific.

Mice immunised subcutaneously or orally with a monoclonal anti-idiotypic antibody to the exoglycolipid antigen developed a protective response to serovar C, though remained susceptible to challenge with serovar K (Whittum-Hudson, J A et al. Nat. Med. 1996 2(10):1116-1121).

One protein which has been disclosed to date and which shows a high level of sequence homology among different serovars, namely class I accessible protein-1 (referred to as Cap1, or Ct-529). Such proteins have potential use in the development of vaccines which stimulate protection against more than one serovar (Fling, S P et al. PNAS 2001 98(3):1160-1165). However, in addition to the requirement for high levels of sequence homology between serovars, proteins of use in vaccines must also elicit sufficient immune response.

Lyons, J M et al. BMC Infectious Diseases 2005 5:105 describes the acquisition of homotypic and heterotypic immunity against oculogenital Chlamydia trachomatis serovars following genital tract infection in mice.

Patel, H C et al. Genitourin. Med. 1995 71:2 94-97 found that patients with dual Chlamydial infection of the conjunctiva and genital tract had a higher IgG titre than those with ocular or genital infection alone.

Ogra, P L et al. Clin. Microbiol. Rev. 2001 14(2):430-445 discusses general vaccination strategies for obtaining mucosal immune responses.

International patent application number PCT/US2006/010793, publication number WO2006/104890, discloses combinations of Chlamydial antigens of use in the prevention and/or treatment of Chlamydial infection, although does not specifically disclose their use in the treatment of ocular infections.

There remains a need in the art for effective methods for the treatment and prevention of ocular Chlamydia trachomatis infections. There also remains a need for effective methods for the treatment and prevention of ocular Chlamydia trachomatis infections resulting from a range of serovars. The present invention fulfils these needs and further provides other related advantages.

The present inventors have surprisingly found administration of certain immunogenic compositions to be an effective method of inducing an immune response which is protective against ocular Chlamydia trachomatis infection.

Furthermore, it has been found that Chlamydia trachomatis proteins Ct-089, Ct-858 and Ct-875 in particular are both highly antigenic and have a high degree of sequence identity across the different Chlamydia trachomatis serovars. There is particularly high conservation in the region of the predicted epitopes. In light of this finding, the possibility exists for the development of vaccines against ocular Chlamydia trachomatis infection which are effective against a broad range of Chlamydia trachomatis serovars (i.e. which may be of use in cross-protection).

### SUMMARY OF THE INVENTION

According to the present invention there is provided a method for the treatment or prevention of ocular Chlamydia trachomatis infection by the administration of a safe and effective amount of an immunogenic composition comprising one or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd).

Additionally provided is an immunogenic composition comprising one or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd), for use in the treatment or prevention of ocular Chlamydia trachomatis infection.

There is also provided the use of one or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd), in the manufacture of an immunogenic composition for the treatment or prevention of ocular Chlamydia trachomatis infection.

According to the present invention there is provided a method for the treatment or prevention of ocular Chlamydia trachomatis infection by the administration of a safe and effective amount of an immunogenic composition comprising two or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd).

Additionally provided is an immunogenic composition comprising two or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd), for use in the treatment or prevention of ocular Chlamydia trachomatis infection.

There is also provided the use of two or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd), in the manufacture of an immunogenic composition for the treatment or prevention of ocular Chlamydia trachomatis infection.

In a further aspect of the present invention, there is provided a method for the treatment or prevention of ocular Chlamydia trachomatis infection by the ocular administration of a safe and effective amount of an immunogenic composition comprising one or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd).

Also provided is a method for the treatment or prevention of ocular Chlamydia trachomatis infection by the non-ocular administration of a safe and effective amount of an immunogenic composition comprising one or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd).

Suitably, the immunogenic composition comprises a Ct-089, Ct-858 or Ct-875 protein, immunogenic fragment thereof or polynucleotide encoding said protein or fragment.

In a further aspect of the present invention there is provided a method for the treatment or prevention of ocular Chlamydia trachomatis infection by a second Chlamydia trachomatis serovar, comprising the administration of an immunogenic composition comprising a protein selected from the list consisting of Ct-089, Ct-858 or Ct-875, an immunogenic fragment thereof or polynucleotide encoding said protein or fragment, which is derived from a first Chlamydia trachomatis serovar.

There is also provided the use of a protein selected from the list consisting of Ct-089, Ct-858 or Ct-875, an immunogenic fragment thereof or polynucleotide encoding said protein or fragment, which is derived from a first Chlamydia trachomatis serovar, in the manufacture of an immunogenic composition for the treatment or prevention of ocular Chlamydia trachomatis infection by a second Chlamydia trachomatis serovar.

Additionally provided is an immunogenic composition comprising a protein selected from the list consisting of Ct-089, Ct-858 or Ct-875, an immunogenic fragment thereof or polynucleotide encoding said protein or fragment, which is derived from a first Chlamydia trachomatis serovar, for use in the treatment or prevention of ocular Chlamydia trachomatis infection by a second Chlamydia trachomatis serovar.

Suitably, the immunogenic composition comprises two or more Chlamydia trachomatis proteins, immunogenic fragments thereof or polynucleotides encoding said proteins or fragments selected from the list consisting of Swib, Momp, Ct-858, Ct-875, Ct-622, Ct-089, passenger domain of PmpG (PmpGpd) and passenger domain of PmpD (PmpDpd) (in particular two or more Chlamydia trachomatis proteins, immunogenic fragments thereof or polynucleotides encoding said proteins or fragments selected from the list consisting of Ct-089, Ct-858 and Ct-875, e.g. Ct-858 and Ct-875). In particular, the immunogenic composition comprises Chlamydia trachomatis proteins, immunogenic fragments thereof or polynucleotides encoding said proteins or fragments, relating to each of Ct-089, Ct-858 and Ct-875.

In a specific embodiment, the immunogenic composition may be formulated as a pharmaceutical composition, further comprising a pharmaceutically acceptable diluent or carrier.

The immunogenicity of the immunogenic composition may be enhanced by formulation as a vaccine composition which further comprises an adjuvant.

### BRIEF DESCRIPTION OF THE FIGURES

FIG. 1 shows the sequence alignment for Ct-089 from Chlamydia trachomatis serovar E with Ct-089 from a range of other Chlamydia trachomatis serovars.

FIGS. 2a and 2b show the sequence alignment for Ct-858 from Chlamydia trachomatis serovar E with Ct-858 from a range of other Chlamydia trachomatis serovars.

FIGS. 3a and 3b show the sequence alignment for Ct-875 from Chlamydia trachomatis serovar E with Ct-875 from a range of other Chlamydia trachomatis serovars.

FIG. 4 shows the results of ocular swabs taken from immunised mice on day 7 following ocular challenge with Chlamydia trachomatis elementary bodies.

FIG. 5 shows the results of ocular swabs taken from immunised mice on day 14 following ocular challenge with Chlamydia trachomatis elementary bodies.

FIG. 6 shows the results of ocular swabs taken from immunised mice on day 21 following ocular challenge with Chlamydia trachomatis elementary bodies.

### DETAILED DESCRIPTION OF THE INVENTION

By the term ‘two or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd)’ is meant comprises at least one component (i.e. protein, immunogenic fragment thereof or polynucleotide encoding said protein or fragment) relating to a first Chlamydial antigen from the aforementioned list and at least one component (i.e. protein, immunogenic fragment thereof or polynucleotide encoding said protein or fragment) relating to a second Chlamydial antigen from the aforementioned list. References to ‘three or more’ and such like are to be construed accordingly.

The following provides polynucleotide and polypeptide sequences for certain antigens which have been listed above and which may be used in the compositions of the invention:

### BRIEF DESCRIPTION OF SEQUENCE IDENTIFIERS

SEQ ID NO: 1 is the cDNA sequence of Ct-460, also known as Swib, from Chlamydia trachomatis serovar LGVII (serovar LGVII is may also be referred to as serovar LII or L2).

SEQ ID NO: 2 is the protein sequence of Ct-460, also known as Swib, from Chlamydia trachomatis serovar LGVII which protein is encoded by SEQ ID NO: 1.

SEQ ID NO: 3 is the cDNA sequence of the Chlamydia antigen known as Major Outer Membrane Protein (Momp) from Chlamydia trachomatis serovar F.

SEQ ID NO: 4 is the protein sequence of the Chlamydia antigen known as Major Outer Membrane Protein (Momp) from Chlamydia trachomatis serovar F, which protein is encoded by SEQ ID NO: 3.

SEQ ID NO: 5 is the cDNA sequence of Ct-858 from Chlamydia trachomatis serovar E.

SEQ ID NO: 6 is the protein sequence of Ct-858 Chlamydia trachomatis serovar E, which protein is encoded by SEQ ID NO: 5.

SEQ ID NO: 7 is the cDNA sequence of Ct-875 from Chlamydia trachomatis serovar E.

SEQ ID NO: 8 is the protein sequence of Ct-875 from Chlamydia trachomatis serovar E, which protein is encoded by SEQ ID NO: 7.

SEQ ID NO: 9 is the cDNA sequence of Ct-622 from Chlamydia trachomatis serovar E.

SEQ ID NO: 10 is the protein sequence of Ct-622 from Chlamydia trachomatis serovar E, which protein is encoded by SEQ ID NO: 9.

SEQ ID NO: 11 is the cDNA sequence of the passenger domain of PmpG also known as Ct-871 from Chlamydia trachomatis serovar LGVII.

SEQ ID NO: 12 is the protein sequence of the passenger domain of PmpG, also known as Ct-871 from Chlamydia trachomatis serovar LGVII, which protein is encoded by SEQ ID NO: 11.

SEQ ID NO: 13 is the cDNA sequence of the passenger domain of PmpD, also known as Ct-812, from Chlamydia trachomatis serovar LGVII.

SEQ ID NO: 14 is the protein sequence of the passenger domain of PmpD, also known as Ct-812, from Chlamydia trachomatis serovar LGVII, which protein is encoded by SEQ ID NO: 13.

SEQ ID NO: 15 is the cDNA sequence of the Ct-089 from Chlamydia trachomatis serovar E.

SEQ ID NO: 16 is the protein sequence of Ct-089 from Chlamydia trachomatis serovar E, which protein is encoded by SEQ ID NO: 15.

SEQ ID NO: 21 is the cDNA sequence of Ct-875 from Chlamydia trachomatis serovar D.

SEQ ID NO: 22 is the protein sequence of Ct-875 from Chlamydia trachomatis serovar D, which protein is encoded by SEQ ID NO: 21.

SEQ ID NO: 27 is the cDNA sequence PmpG also known as Ct-871 from Chlamydia trachomatis serovar D.

SEQ ID NO: 28 is the protein sequence of PmpG, also known as Ct-871 from Chlamydia trachomatis serovar D, which protein is encoded by SEQ ID NO: 27.

SEQ ID NO: 33 is the cDNA sequence of Ct-858 from Chlamydia trachomatis serovar D.

SEQ ID NO: 34 is the protein sequence of Ct-858 Chlamydia trachomatis serovar D, which protein is encoded by SEQ ID NO: 33.

SEQ ID NO: 41 is the cDNA sequence of PmpD, also known as Ct-812, from Chlamydia trachomatis serovar D.

SEQ ID NO: 42 is the protein sequence of PmpD, also known as Ct-812, from Chlamydia trachomatis serovar D, which protein is encoded by SEQ ID NO: 41. The passenger domain spans amino acids 31 to 1203.

SEQ ID NO: 47 is the cDNA sequence of the Chlamydia antigen known as Major Outer Membrane Protein (Momp), also known as Ct-681 from Chlamydia trachomatis serovar LGVII.

SEQ ID NO: 48 is the protein sequence of the Chlamydia antigen known as Major Outer Membrane Protein (Momp), also known as Ct-681 from Chlamydia trachomatis serovar LGVII, which protein is encoded by SEQ ID NO: 47.

SEQ ID NO: 49 is the cDNA sequence of the Chlamydia antigen known as Major Outer Membrane Protein (Momp), also known as Ct-681 from Chlamydia trachomatis serovar J.

SEQ ID NO: 50 is the protein sequence of the Chlamydia antigen known as Major Outer Membrane Protein (Momp), also known as Ct-681 from Chlamydia trachomatis serovar J, which protein is encoded by SEQ ID NO: 49.

SEQ ID NO: 51 is the cDNA sequence of the Chlamydia antigen known as Major Outer Membrane Protein (Momp), also known as Ct-681 from Chlamydia trachomatis serovar H.

SEQ ID NO: 52 is the protein sequence of the Chlamydia antigen known as Major Outer Membrane Protein (Momp), also known as Ct-681 from Chlamydia trachomatis serovar H, which protein is encoded by SEQ ID NO: 51.

SEQ ID NO: 53 is the cDNA sequence of the Chlamydia antigen known as Major Outer Membrane Protein (Momp), also known as Ct-681 from Chlamydia trachomatis serovar E.

SEQ ID NO: 54 is the protein sequence of the Chlamydia antigen known as Major Outer Membrane Protein (Momp), also known as Ct-681 from Chlamydia trachomatis serovar E, which protein is encoded by SEQ ID NO: 53.

SEQ ID NO: 55 is the cDNA sequence of the Chlamydia antigen known as Major Outer Membrane Protein (Momp), also known as Ct-681 from Chlamydia trachomatis serovar D.

SEQ ID NO: 56 is the protein sequence of the Chlamydia antigen known as Major Outer Membrane Protein (Momp), also known as Ct-681 from Chlamydia trachomatis serovar D, which protein is encoded by SEQ ID NO: 55.

SEQ ID NO: 57 is the cDNA sequence of Ct-622 from Chlamydia trachomatis serovar D.

SEQ ID NO: 58 is the protein sequence of Ct-622 from Chlamydia trachomatis serovar D, which protein is encoded by SEQ ID NO: 57.

SEQ ID NO: 63 is the cDNA sequence of Ct-460, also known as Swib from Chlamydia trachomatis serovar D.

SEQ ID NO: 64 is the protein sequence of Ct-460, also known as Swib from Chlamydia trachomatis serovar D, which protein is encoded by SEQ ID NO: 63.

SEQ ID NO: 71 is the cDNA sequence of Ct-089 from Chlamydia trachomatis serovar D.

SEQ ID NO: 72 is the protein sequence of Ct-089 from Chlamydia trachomatis serovar D, which protein is encoded by SEQ ID NO: 71.

SEQ ID NO: 79 is the cDNA sequence of the Ct-089 from Chlamydia trachomatis serovar A.

SEQ ID NO: 80 is the protein sequence of Ct-089 from Chlamydia trachomatis serovar A, which protein is encoded by SEQ ID NO: 79.

SEQ ID NO: 81 is the cDNA sequence of Ct-089 from Chlamydia trachomatis serovar B.

SEQ ID NO: 82 is the protein sequence of Ct-089 from Chlamydia trachomatis serovar B, which protein is encoded by SEQ ID NO: 81.

SEQ ID NO: 83 is the cDNA sequence of Ct-089 from Chlamydia trachomatis serovar G.

SEQ ID NO: 84 is the protein sequence of Ct-089 from Chlamydia trachomatis serovar G, which protein is encoded by SEQ ID NO: 83.

SEQ ID NO: 85 is the cDNA sequence of Ct-089 from Chlamydia trachomatis serovar H.

SEQ ID NO: 86 is the protein sequence of Ct-089 from Chlamydia trachomatis serovar H, which protein is encoded by SEQ ID NO: 85.

SEQ ID NO: 87 is the cDNA sequence of Ct-089 from Chlamydia trachomatis serovar I.

SEQ ID NO: 88 is the protein sequence of Ct-089 from Chlamydia trachomatis serovar I, which protein is encoded by SEQ ID NO: 87.

SEQ ID NO: 89 is the cDNA sequence of Ct-089 from Chlamydia trachomatis serovar J.

SEQ ID NO: 90 is the protein sequence of Ct-089 from Chlamydia trachomatis serovar J, which protein is encoded by SEQ ID NO: 89.

SEQ ID NO: 91 is the cDNA sequence of Ct-089 from Chlamydia trachomatis serovar K.

SEQ ID NO: 92 is the protein sequence of Ct-089 from Chlamydia trachomatis serovar K, which protein is encoded by SEQ ID NO: 91.

SEQ ID NO: 93 is the cDNA sequence of Ct-089 from Chlamydia trachomatis serovar L2.

SEQ ID NO: 94 is the protein sequence of Ct-089 from Chlamydia trachomatis serovar L2, which protein is encoded by SEQ ID NO: 93.

SEQ ID NO: 95 is the cDNA sequence of Ct-858 from Chlamydia trachomatis serovar A.

SEQ ID NO: 96 is the protein sequence of Ct-858 from Chlamydia trachomatis serovar A, which protein is encoded by SEQ ID NO: 95.

SEQ ID NO: 97 is the cDNA sequence of Ct-858 from Chlamydia trachomatis serovar B.

SEQ ID NO: 98 is the protein sequence of Ct-858 from Chlamydia trachomatis serovar B, which protein is encoded by SEQ ID NO: 97.

SEQ ID NO: 99 is the cDNA sequence of Ct-858 from Chlamydia trachomatis serovar G.

SEQ ID NO: 100 is the protein sequence of Ct-858 from Chlamydia trachomatis serovar G, which protein is encoded by SEQ ID NO: 99.

SEQ ID NO: 101 is the cDNA sequence of Ct-858 from Chlamydia trachomatis serovar H.

SEQ ID NO: 102 is the protein sequence of Ct-858 from Chlamydia trachomatis serovar H, which protein is encoded by SEQ ID NO: 101.

SEQ ID NO: 103 is the cDNA sequence of Ct-858 from Chlamydia trachomatis serovar I.

SEQ ID NO: 104 is the protein sequence of Ct-858 from Chlamydia trachomatis serovar I, which protein is encoded by SEQ ID NO: 103.

SEQ ID NO: 105 is the cDNA sequence of Ct-858 from Chlamydia trachomatis serovar J.

SEQ ID NO: 106 is the protein sequence of Ct-858 from Chlamydia trachomatis serovar J, which protein is encoded by SEQ ID NO: 105.

SEQ ID NO: 107 is the cDNA sequence of Ct-858 from Chlamydia trachomatis serovar K.

SEQ ID NO: 108 is the protein sequence of Ct-858 from Chlamydia trachomatis serovar K, which protein is encoded by SEQ ID NO: 107.

SEQ ID NO: 109 is the cDNA sequence of Ct-858 from Chlamydia trachomatis serovar L2.

SEQ ID NO: 110 is the protein sequence of Ct-858 from Chlamydia trachomatis serovar L2, which protein is encoded by SEQ ID NO: 109.

SEQ ID NO: 111 is the cDNA sequence of Ct-875 from Chlamydia trachomatis serovar A.

SEQ ID NO: 112 is the protein sequence of Ct-875 from Chlamydia trachomatis serovar A, which protein is encoded by SEQ ID NO: 111.

SEQ ID NO: 113 is the cDNA sequence of Ct-875 from Chlamydia trachomatis serovar B.

SEQ ID NO: 114 is the protein sequence of Ct-875 from Chlamydia trachomatis serovar B, which protein is encoded by SEQ ID NO: 113.

SEQ ID NO: 115 is the cDNA sequence of Ct-875 from Chlamydia trachomatis serovar G.

SEQ ID NO: 116 is the protein sequence of Ct-875 from Chlamydia trachomatis serovar G, which protein is encoded by SEQ ID NO: 115.

SEQ ID NO: 117 is the cDNA sequence of Ct-875 from Chlamydia trachomatis serovar H.

SEQ ID NO: 118 is the protein sequence of Ct-875 from Chlamydia trachomatis serovar H, which protein is encoded by SEQ ID NO: 117.

SEQ ID NO: 119 is the cDNA sequence of Ct-875 from Chlamydia trachomatis serovar I.

SEQ ID NO: 120 is the protein sequence of Ct-875 from Chlamydia trachomatis serovar I, which protein is encoded by SEQ ID NO: 119.

SEQ ID NO: 121 is the cDNA sequence of Ct-875 from Chlamydia trachomatis serovar J.

SEQ ID NO: 122 is the protein sequence of Ct-875 from Chlamydia trachomatis serovar J, which protein is encoded by SEQ ID NO: 121.

SEQ ID NO: 123 is the cDNA sequence of Ct-875 from Chlamydia trachomatis serovar K.

SEQ ID NO: 124 is the protein sequence of Ct-875 from Chlamydia trachomatis serovar K, which protein is encoded by SEQ ID NO: 123.

SEQ ID NO: 125 is the cDNA sequence of Ct-875 from Chlamydia trachomatis serovar L2.

SEQ ID NO: 126 is the protein sequence of Ct-875 from Chlamydia trachomatis serovar L2, which protein is encoded by SEQ ID NO: 125.

Certain of the above sequences and other related Chlamydia polypeptides and polynucleotides from a number of serovars are known and available in the art. Further related sequences can be found in issued U.S. Pat. Nos. 6,447,779, 6,166,177, 6,565,856, 6,555,115, 6,432,916, and 6,448,234 and are also disclosed in U.S. patent applications Nos. 10/197,220, 10/762,058 and 10/872,155, each of which is herein incorporated by reference.

The sequence of Ct-089 from serovar D and the potential application of this protein as an antigen has been publicly disclosed, for example in WO02/08267 (Corixa Corporation). The sequence of Ct-089 from serovar L2 was disclosed in WO99/28475 (Genset). The role of CopN (also known as Ct-089) as a putative exported regulator of type III protein secretion systems is discussed in Fields, K A and Hackstadt, T Mol. Microbiol. 2000 38(5):1048-1060. The sequences of Ct-858 and Ct-875 from serovar D are available from the Swiss-Prot database, primary accession numbers 084866 and 084883 respectively. For further information see Stephens, R S et al. Science 1998 282:754-759. The use of Ct-858 as an antigen is disclosed, for example, in WO02/08267 (Corixa Corporation). The sequence of Ct-875 from serovar E (incorporating a His-tag) and its use as an antigen is disclosed, for example, in US 20040137007. However, the document incorrectly refers to sequence number 139 as being Ct-875, when it is in fact sequence number 140 therein.

Suitably the immunogenic composition of use in the present invention will comprise three or more Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd), for example three, four, five or six Chlamydia trachomatis proteins, immunogenic fragments thereof or polynucleotides encoding said proteins or fragments, selected from the list consisting of Swib, Momp, Ct-858, Ct-875, Ct-622, Ct-089, passenger domain of PmpG (PmpGpd) and passenger domain of PmpD (PmpDpd).

One skilled in the art will recognise that the each component in an immunogenic composition may independently be a protein, immunogenic fragment thereof or polynucleotide encoding said protein or fragment. Additionally, one skilled in the art will recognise that a number of proteins or immunogenic fragments thereof may be contained within a single fusion protein and need not be provided separately (and correspondingly a number of polynucleotides encoding specific proteins and/or immunogenic fragments thereof may be contained within a single polynucleotide sequence, for example a polynucleotide sequence encoding a fusion protein). In one embodiment of the invention all of the Chlamydia trachomatis proteins, immunogenic fragments thereof or polynucleotides encoding said proteins or fragments are provided as polypeptides (such as a single fusion protein). In a second embodiment of the invention all of the Chlamydia trachomatis proteins, immunogenic fragments thereof or polynucleotides encoding said proteins or fragments are provided as polynucleotides (for example a single polynucleotide sequence, such as a polynucleotide sequence encoding a fusion protein). It will be recognised that a polypeptide component (i.e. a protein or immunogenic fragment thereof) may be comprised within a larger polypeptide which contains additional residues. Similarly, a polynucleotide encoding a protein or immunogenic fragment thereof may be comprised within a larger polynucleotide.

In addition to the proteins, immunogenic fragments thereof or polynucleotides encoding said proteins or fragments selected from the list consisting of Swib, Momp, Ct-858, Ct-875, Ct-622, Ct-089, passenger domain of PmpG (PmpGpd) and passenger domain of PmpD (PmpDpd), the immunogenic compositions may comprise other proteins, immunogenic fragments thereof or polynucleotides encoding said proteins or fragments relating to any other Chlamydial antigen (for example proteins, immunogenic fragments thereof or polynucleotides encoding said proteins or fragments relating to one, two or three other Chlamydial antigens).

In order to obtain effective immune responses across a diverse out-bred human population, it is advantageous to utilise combinations of antigens. Not all antigen combinations are complementary. Certain combinations of antigens have been found by the present inventors to have broad recognition by human subjects with a history of Chlamydial infection.

Suitably, the immunogenic composition comprises a Ct-089, Ct-858 or Ct-875 protein, immunogenic fragment thereof or polynucleotide encoding said protein or fragment. More suitably, the immunogenic composition comprises two or more Chlamydia trachomatis proteins, immunogenic fragments thereof or polynucleotides encoding said proteins or fragments, selected from the list consisting of Ct-089, Ct-858 and Ct-875 (e.g. Ct-089 and Ct-858; Ct-089 and Ct-875; or Ct-858 and Ct-875). In particular, the immunogenic composition may comprise Chlamydia trachomatis proteins, immunogenic fragments thereof or polynucleotides encoding said proteins or fragments, relating to each of Ct-089, Ct-858 and Ct-875.

For example, the immunogenic composition may comprise components relating to one of the following combinations, provided that all of the combinations comprise Ct-858 and Ct-875 components:
1. Five out of: Swib, Momp, PmpDpd, Ct-858, PmpGpd and Ct-875
2. Three out of: PmpDpd, Ct-858, Ct-0875, Swib
3. Five out of: Momp, PmpDpd, Ct-858, Ct-622, Ct-875 and Swib
4. Five out of: Momp, PmpDpd, Ct-858, PmpGpd, Ct-622 and Ct-875
5. Three out of: Ct-858, Ct-875, Ct-622 and Ct-089
6. Three out of: PmpDpd, Ct-858, Ct-875, Ct-089
7. Four out of: Momp, PmpD, Ct-858, PmpGpd and Ct-875

Specific immunogenic compositions may comprise components relating to one of the following combinations (which each contain Ct-858 and Ct-875 components):
1a. Momp, PmpDpd, Ct-858, Ct-875, Swib, Ct-089
2a. PmpDpd, Ct-858, Ct-875, Swib, Ct-089
3a. Momp, PmpDpd, Ct-858, Ct-622, Ct-875, Swib, Ct-089
4a. Momp, PmpDpd, Ct-858, PmpGpd, Ct-622, Ct-875, Ct-089
5a. Ct-858, Ct-875
6a. Momp, Ct-858, Ct-875, Ct-089
7a. Momp, Ct-858, Ct-875
8a. Momp, PmpD, Ct-858, PmpGpd, Ct-875, Ct-089
9a. PmpDpd, Ct-858, Ct-875, Ct-089

An alternative immunogenic composition may comprise components relating to Momp, Ct-089, Ct-858, Swib and PmpDpd.

The immunogenic compositions of use in the present invention may be administered by any appropriate vaccination route. In one embodiment of the invention the immunogenic composition is administered ocularly. In a second embodiment of the invention the immunogenic composition is administered non-ocularly.

Non-ocular administration routes include administration via mucosal surfaces other than the eye. In one embodiment of the invention non-ocular administration is via a mucosal surface (e.g. intranasal, oral or vaginal). In a second embodiment of the invention non-ocular administration is via injection (e.g. intradermal injection, subcutaneous injection, intramuscular injection or intravenous injection, in particular intramuscular injection).

In a further aspect of the present invention there is provided a method for the treatment or prevention of ocular Chlamydia trachomatis infection by a second Chlamydia trachomatis serovar, comprising the administration of an immunogenic composition comprising a protein selected from the list consisting of Ct-089, Ct-858 or Ct-875, an immunogenic fragment thereof or polynucleotide encoding said protein or fragment, which is derived from a first Chlamydia trachomatis serovar.

There is also provided the use of a protein selected from the list consisting of Ct-089, Ct-858 or Ct-875, an immunogenic fragment thereof or polynucleotide encoding said protein or fragment, which is derived from a first Chlamydia trachomatis serovar, in the manufacture of an immunogenic composition for the treatment or prevention of ocular Chlamydia trachomatis infection by a second Chlamydia trachomatis serovar.

Additionally provided is an immunogenic composition comprising a protein selected from the list consisting of Ct-089, Ct-858 or Ct-875, an immunogenic fragment thereof or polynucleotide encoding said protein or fragment, which is derived from a first Chlamydia trachomatis serovar, for use in the treatment or prevention of ocular Chlamydia trachomatis infection by a second Chlamydia trachomatis serovar.

In one embodiment of the invention the immunogenic composition of use in cross-protection comprises one protein, immunogenic fragment thereof or polynucleotide encoding said protein or fragment, selected from the list consisting of Ct-089, Ct-858 and Ct-875. Immunogenic compositions which comprise only one protein, immunogenic fragment thereof or polynucleotide encoding said protein or fragment, selected from the list consisting of Ct-089, Ct-858 and Ct-875 will suitably further comprise at least one additional Chlamydial antigen (for example one, two, three or four additional antigens).

In a second embodiment of the invention the immunogenic composition of use in cross-protection comprises two proteins, immunogenic fragments thereof or polynucleotides encoding said proteins or fragments, selected from the list consisting of Ct-089, Ct-858 and Ct-875. For example: Ct-089 and Ct-858; Ct-089 and Ct-875; or Ct-858 and Ct-875. Such compositions may further comprise additional Chlamydial antigens (for example one, two or three additional antigens).

In a third embodiment of the invention the immunogenic composition of use in cross-protection comprises three proteins, immunogenic fragments thereof or polynucleotides encoding said proteins or fragments, selected from the list consisting of Ct-089, Ct-858 and Ct-875. Such compositions also may further comprise additional Chlamydial antigens (for example one or two additional antigens).

Typically, additional Chlamydial antigens (which may be in the form of proteins, immunogenic fragments thereof or polynucleotides encoding said proteins or fragments) of use in immunogenic compositions of use in cross-protection will be selected from the list consisting of Swib, Momp, Ct-622, passenger domain of PmpG (PmpGpd) and passenger domain of PmpD (PmpDpd), in particular from Swib, Momp and passenger domain of PmpD.

The first Chlamydia trachomatis serovar may be any Chlamydia trachomatis serovar. The second Chlamydia trachomatis serovar may be any Chlamydia trachomatis serovar, excluding that of the first Chlamydia trachomatis serovar.

In one embodiment of the invention the first Chlamydia trachomatis serovar is selected from the list consisting of Chlamydia trachomatis serovars A, B, Ba, C, D, Da, E, F, G, H, I, Ia, J, Ja, K, L1, L2 and L3. In a second embodiment of the invention the first Chlamydia trachomatis serovar is selected from the Chlamydia trachomatis ocular serovars (for example A, B, Ba and C). In another embodiment of the invention the first Chlamydia trachomatis serovar is selected from the Chlamydia trachomatis oculogenital serovars (for example D, Da, E, F, G, H, I, Ia, J, Ja and K). In a further embodiment of the invention the first Chlamydia trachomatis serovar is selected from the Chlamydia trachomatis LGV serovars (for example L1, L2 and L3).

In one embodiment of the invention the second Chlamydia trachomatis serovar is selected from the list consisting of Chlamydia trachomatis serovars A, B, Ba, C, D, Da, E, F, G, H, I, Ia, J, Ja, K, L1, L2 and L3. In a second embodiment of the invention the second Chlamydia trachomatis serovar is selected from the Chlamydia trachomatis ocular serovars (for example A, B, Ba and C). In another embodiment of the invention the second Chlamydia trachomatis serovar is selected from the Chlamydia trachomatis oculogenital serovars (for example D, Da, E, F, G, H, I, Ia, J, Ja and K). In a further embodiment of the invention the second Chlamydia trachomatis serovar is selected from the Chlamydia trachomatis LGV serovars (for example L1, L2 and L3).

In order to maximise the breadth of action of the cross-protection methods and uses, it may be desirable that the first Chlamydia trachomatis serovar is selected such that there is a high level of sequence identity (for example at least 90%, especially at least 95%, in particular at least 98%, more particularly at least 99% sequence identity) with the majority of other Chlamydia trachomatis serovars (for example at least 50%, especially at least 70%, in particular at least 80%, more particularly at least 90% of other Chlamydia trachomatis serovars).

In order to maximise the practical application of the method and use of the present invention, it may be desirable that the first Chlamydia trachomatis serovar is selected such that there is a high level of sequence identity (for example at least 90%, especially at least 95%, in particular at least 98%, more particularly at least 99% sequence identity) with the majority (for example at least 50%, especially at least 70%, in particular at least 80%, more particularly at least 90%) of common Chlamydia trachomatis serovars (such as the common ocular serovars, the common oculogenital serovars, the common LGV serovars, or a combination of any two of these serovar groups, for example, the common ocular and oculogentical serovars). Common Chlamydia trachomatis ocular serovars include A and B. Common Chlamydia trachomatis oculogenital serovars include D, E, F and I (Lan, J et al. J. Clin. Microbiol. 1995 33(12):3194-3197; Singh, V et al. J. Clin. Microbiol. 2003 41(6):2700-2702). Common Chlamydia trachomatis LGV serovars include L2.

In one embodiment of the present invention the first Chlamydia trachomatis serovar is Chlamydia trachomatis serovar E.

In one embodiment of the invention the second Chlamydia trachomatis serovar is selected from Chlamydia trachomatis serovars A, B and K.

In one example of the present invention, where the immunogenic composition comprises Ct-089 protein, immunogenic fragment thereof or polynucleotide encoding said protein or fragment, derived from Chlamydia trachomatis serovar E, the immunogenic composition may be used in the treatment or prophylaxis of infections arising from Chlamydia trachomatis serovars A, B, D, G, H, I, J, K or L2; in particular A, B, D, G, H, I or K; especially A or B.

In a second example of the present invention, where the immunogenic composition comprises Ct-858 protein, immunogenic fragment thereof or polynucleotide encoding said protein or fragment, an immunogenic fragment thereof or polynucleotide encoding it, derived from Chlamydia trachomatis serovar E, the immunogenic composition may be used in the treatment or prophylaxis of infections arising from Chlamydia trachomatis serovars A, B, D, G, H, I, J, K or L2; in particular J or L2.

In a further example of the present invention, where the immunogenic composition comprises Ct-875 protein, immunogenic fragment thereof or polynucleotide encoding said protein or fragment, an immunogenic fragment thereof or polynucleotide encoding it, derived from Chlamydia trachomatis serovar E, the immunogenic composition may be used in the treatment or prophylaxis of infections arising from Chlamydia trachomatis serovars A, B, D, G, H, I, J, K or L2; in particular A, B, D, G, H, I or K.

The first and second Chlamydia trachomatis serovars may be associated with the same disease state (for example they may both be ocular serovars or both be oculogenital serovars), or the first and second Chlamydia trachomatis serovars may be associated with different disease states (for example the first Chlamydia trachomatis serovar may an oculogenital serovar and the second Chlamydia trachomatis serovar may be an ocular serovar, or vice versa).

In the event that the immunogenic composition of use in the present invention comprises more than one protein, immunogenic fragment thereof or polynucleotide encoding said protein or fragment, selected from the list consisting of Ct-089, Ct-858 and Ct-875, it should be noted that each protein, immunogenic fragment thereof or polynucleotide encoding them, may optionally be derived from a different first Chlamydia trachomatis serovar which may be independently selected. Although, one skilled in the art will recognise that the immunogenic compositions may also include additional Ct-089, Ct-858 and Ct-875 proteins, immunogenic fragments thereof or polynucleotides encoding said proteins or fragments which are derived from the second Chlamydia trachomatis serovar.

Thus the immunogenic compositions of use in the present invention may employ the polypeptide sequences provided in the sequence listing or variants thereof, immunogenic fragments of these, or polynucleotide sequences encoding these (which may be, for example, the polynucleotide sequences provided in the sequence listing or fragments of these which encode immunogenic fragments of the polypeptides).

The protein antigens described herein may be in the form of fusion proteins. The fusion proteins may also contain additional polypeptides, optionally heterologous peptides from Chlamydia or other sources. Antigens within fusion sequences may be modified, for example, by adding linker peptide sequences as described below. These linker peptides may be inserted between one or more polypeptides which make up each of the fusion proteins. The antigens described herein may also be in the form of chemical conjugates.

It will be evident that in the case of the passenger domains of PmpD and PmpG, these may be present in the context of a larger portion of the PmpD or PmpG protein or polynucleotide, for example full length PmpD or PmpG or a fragment thereof, provided that the fragment comprises the passenger domain.

In particular embodiments:

(i) the Ct-089 component will typically be a polypeptide having at least 90% homology (for example 95% homology) to a Ct-089 sequence provided in the sequence listing herein, an immunogenic fragment thereof, or a polynucleotide having at least 90% homology (for example 95% homology) to a Ct-089 sequence provided in the sequence listing herein, or a fragment thereof which encodes an immunogenic fragment of the corresponding protein. In particular, the Ct-089 component will be derived from Chlamydia trachomatis serovar E.

(ii) the Ct-858 component will typically be a polypeptide having at least 90% homology (for example 95% homology) to a Ct-858 sequence provided in the sequence listing herein, an immunogenic fragment thereof, or a polynucleotide having at least 90% homology (for example 95% homology) to a Ct-858 sequence provided in the sequence listing herein, or a fragment thereof which encodes an immunogenic fragment of the corresponding protein. In particular, the Ct-858 component will be derived from Chlamydia trachomatis serovar E.

(iii) the Ct-875 component will typically be a polypeptide having at least 90% homology (for example 95% homology) to a Ct-875 sequence provided in the sequence listing herein, an immunogenic fragment thereof, or a polynucleotide having at least 90% homology (for example 95% homology) to a Ct-875 sequence provided in the sequence listing herein, or a fragment thereof which encodes an immunogenic fragment of the corresponding protein. In particular, the Ct-875 component will be derived from Chlamydia trachomatis serovar E.

(iv) the PmpDpd component will typically be a polypeptide having at least 90% homology (for example 95% homology) to a PmpDpd sequence provided in the sequence listing herein, an immunogenic fragment thereof, or a polynucleotide having at least 90% homology (for example 95% homology) to a PmpDpd sequence provided in the sequence listing herein, or a fragment thereof which encodes an immunogenic fragment of the corresponding protein. In particular, the PmpDpd component will be derived from Chlamydia trachomatis serovar LII.

(v) the PmpGpd component will typically be a polypeptide having at least 90% homology (for example 95% homology) to a PmpGpd sequence provided in the sequence listing herein, an immunogenic fragment thereof, or a polynucleotide having at least 90% homology (for example 95% homology) to a PmpGpd sequence provided in the sequence listing herein, or a fragment thereof which encodes an immunogenic fragment of the corresponding protein. In particular, the PmpGpd component will be derived from Chlamydia trachomatis serovar LII.

(vi) the Momp component will typically be a polypeptide having at least 90% homology (for example 95% homology) to a Momp sequence provided in the sequence listing herein, an immunogenic fragment thereof, or a polynucleotide having at least 90% homology (for example 95% homology) to a Momp sequence provided in the sequence listing herein, or a fragment thereof which encodes an immunogenic fragment of the corresponding protein. In particular, the Momp component will be derived from Chlamydia trachomatis serovar F.

(vii) the Swib component will typically be a polypeptide having at least 90% homology (for example 95% homology) to a Swib sequence provided in the sequence listing herein, an immunogenic fragment thereof, or a polynucleotide having at least 90% homology (for example 95% homology) to a Swib sequence provided in the sequence listing herein, or a fragment thereof which encodes an immunogenic fragment of the corresponding protein. In particular, the Swib component will be derived from Chlamydia trachomatis serovar LII.

(viii) the Ct-622 component will typically be a polypeptide having at least 90% homology (for example 95% homology) to a Ct-622 sequence provided in the sequence listing herein, an immunogenic fragment thereof, or a polynucleotide having at least 90% homology (for example 95% homology) to a Ct-622 sequence provided in the sequence listing herein, or a fragment thereof which encodes an immunogenic fragment of the corresponding protein. In particular, the Ct-622 component will be derived from Chlamydia trachomatis serovar E.

The immunogenic compositions of use in the present invention may further comprise other components designed to enhance the antigenicity of the antigens or to improve these antigens in other aspects, for example, the isolation of these antigens through addition of a stretch of histidine residues at one end of the antigen. The addition of a stretch of histidine residues at one end of the antigen may also improve expression. The immunogenic compositions of use in the invention can comprise additional copies of antigens, or additional polypeptides or polynucleotides from Chlamydia sp. The immunogenic compositions can also comprise additional heterologous polypeptides or polynucleotides from other non-Chlamydia sources. For example, the compositions of the invention can include polypeptides or nucleic acids encoding polypeptides, wherein the polypeptide enhances expression of the antigen, e.g., NS1, an influenza virus protein, or an immunogenic portion thereof (see, e.g. WO99/40188 and WO93/04175). The nucleic acids of the invention can be engineered based on codon preference in a species of choice, e.g., humans. Where the protein sequence for an antigen begins with a Met residue, it will be recognised that this residue can typically be omitted without detriment to the functional properties of the antigen.

### DEFINITIONS

“Fusion polypeptide” or “fusion protein” refers to a protein having at least two Chlamydia polypeptides (which may be the same, or may be different) covalently linked, either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. This term also refers to conservatively modified variants, polymorphic variants, alleles, mutants, subsequences, interspecies homologs, and immunogenic fragments of the antigens that make up the fusion protein. Fusion proteins of use in the invention can also comprise additional copies of a component antigen or immunogenic fragment thereof.

A polynucleotide sequence encoding a fusion protein hybridizes under stringent conditions to at least two nucleotide sequences, each encoding an antigen polypeptide selected from the group consisting of Ct-681 (Momp) or an immunogenic fragment thereof, Ct-871 (PmpG) or an immunogenic fragment thereof, Ct-812 (PmpD) or an immunogenic fragment thereof, Ct-089 or an immunogenic fragment thereof, Ct-858 or an immunogenic fragment thereof, Ct-875 or an immunogenic fragment thereof, Ct-460 (Swib) or an immunogenic fragment thereof, and Ct-622 or an immunogenic fragment thereof. The polynucleotide sequences encoding the individual antigens of the fusion polypeptide therefore include conservatively modified variants, polymorphic variants, alleles, mutants, subsequences, immunogenic fragments, and interspecies homologs of Ct-681 (Momp), Ct-871 (PmpG), Ct-812 (PmpD), Ct-089, Ct-858, Ct-875, Ct-460 (Swib), and Ct-622. The polynucleotide sequences encoding the individual polypeptides of the fusion protein can be in any order.

In some embodiments, the individual polypeptides of the fusion protein are in order (N- to C-terminus) from large to small. Large antigens are approximately 30 to 150 kD in size, medium antigens are approximately 10 to 30 kD in size, and small antigens are approximately less than 10 kD in size.

The sequence encoding the individual polypeptide may be as small as, e.g., an immunogenic fragment such as an individual CTL epitope encoding about 8 to 9 amino acids, or, e.g., an HTL or B cell epitope. The fragment may also include multiple epitopes. The T-helper cell epitopes are peptides bound to HLA class II molecules and recognized by T-helper cells. The prediction of putative T-helper cell epitopes may be performed using the TEPITOPE method described by Sturniolo et al. Nature Biotech. 1999 17:555-561.

A fusion polypeptide specifically binds to antibodies raised against at least two antigen polypeptides selected from Ct-681 (Momp) or an immunogenic fragment thereof, Ct-871 (PmpG) or an immunogenic fragment thereof (e.g. PmpGpd or an immunogenic fragment thereof), Ct-812 (PmpD) or an immunogenic fragment thereof (e.g. PmpDpd or an immunogenic fragment thereof), Ct-089 or an immunogenic fragment thereof, Ct-858 or an immunogenic fragment thereof, Ct-875 or an immunogenic fragment thereof, Ct-460 (Swib) or an immunogenic fragment thereof, and Ct-622 or an immunogenic fragment thereof. The antibodies can be polyclonal or monoclonal. Optionally, the fusion polypeptide specifically binds to antibodies raised against the fusion junction of the antigens, which antibodies do not bind to the antigens individually, i.e., when they are not part of a fusion protein. The fusion polypeptides optionally comprise additional polypeptides, e.g., three, four, five, six, or more polypeptides, up to about 25 polypeptides, optionally heterologous polypeptides or repeated homologous polypeptides, fused to the at least two antigens. The additional polypeptides of the fusion protein are optionally derived from Chlamydia as well as other sources, such as other bacterial, viral, or invertebrate, vertebrate, or mammalian sources. The individual polypeptides of the fusion protein can be in any order. As described herein, the fusion protein can also be linked to other molecules, including additional polypeptides. The compositions of use in the invention can also comprise additional polypeptides that are unlinked to the fusion proteins of the invention. These additional polypeptides may be heterologous or homologous polypeptides.

The term “fused” refers to the covalent linkage between two polypeptides in a fusion protein. The polypeptides are typically joined via a peptide bond, either directly to each other or via an amino acid linker. Optionally, the peptides can be joined via non-peptide covalent linkages known to those of skill in the art.

“FL” refers to full-length, i.e., a polypeptide that is the same length as the wild-type polypeptide.

The term “immunogenic fragment thereof” refers to a polypeptide comprising an epitope that is recognised by T lymphocytes, in particular cytotoxic T lymphocytes, helper T lymphocytes or B cells. Methods of determining epitope regions of a sequence are described elsewhere herein. Suitably, the immunogenic fragment will comprise at least 30%, suitably at least 50%, especially at least 75% and in particular at least 90% (e.g. 95% or 98%) of the amino acids in the reference sequence. Alternatively, the immunogenic fragment will comprise a stretch of at least 9, suitably at least 15 (for example at least 25 or at least 50, in particular at least 100) residues. The immunogenic fragment will suitably comprise all of the epitope regions of the reference sequence.

An adjuvant refers to the components in a vaccine or therapeutic composition that increase the specific immune response to the antigen (see, e.g., Edelman, AIDS Res. Hum Retroviruses 8:1409-1411 (1992)). Adjuvants induce immune responses of the Th1-type and Th-2 type response. Th1-type cytokines (e.g., IFN-γ, IL-2, and IL-12) tend to favour the induction of cell-mediated immune response to an administered antigen, while Th-2 type cytokines (e.g., IL-4, IL-5, Il-6, IL-10 and TNF-β tend to favour the induction of humoral immune responses. Any of a variety of adjuvants may be employed in the vaccines of this invention to enhance the immune response. Some adjuvants contain a substance designed to protect the antigen from rapid catabolism, such as metallic salt particles (e.g. aluminium hydroxide or aluminium phosphate) or mineral oil, and a specific or nonspecific stimulator of immune responses, such as lipid A, Bortadella pertussis or Mycobacterium tuberculosis. Suitable adjuvants are commercially available and include, for example, Freund's Incomplete Adjuvant and Freund's Complete Adjuvant (Difco Laboratories) and Merck Adjuvant 65 (Merck and Company, Inc., Rahway, N.J.). Other suitable adjuvants include monophosphoryl lipid A, 3D-MPL, saponins (e.g. Quil A, in particular the fraction of Quil A known as QS21, especially together with detoxifying components such as cholesterol which are described in WO96/033739), liposome formulations including SBAS1, oil in water emulsions including SBAS2 (Ling et al. Vaccine 1997 15:1562-1567) and CpG oligonucleotide (WO96/02555). Suitable adjuvants for use in the invention are discussed in more detail below.

“Nucleic acid” refers to deoxyribonucleotides or ribonucleotides and polymers thereof in either single- or double-stranded form. The term may also extend to encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Variants” or “Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognise that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

A polynucleotide of the invention may contain a number of silent variations (for example, 1-10, such as 1-5, in particular 1 or 2, and especially 1 codon(s) may be altered) when compared to the reference sequence. A polynucleotide of the invention may contain a number of non-silent conservative variations (for example, 1-10, such as 1-5, in particular 1 or 2, and especially 1 codon(s) may be altered) when compared to the reference sequence. Those skilled in the art will recognise that a particular polynucleotide sequence may contain both silent and non-silent conservative variations.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a functionally similar amino acid or the deletion/addition of residues which do not substantially impact the biological function of the variant. Conservative substitution tables providing functionally similar amino acids are well known in the art. Such conservatively modified variants are in addition to and do not exclude polymorphic variants, interspecies homologs, and alleles of the invention.

A polypeptide of the invention may contain a number of conservative variations (for example, 1-10, such as 1-5, in particular 1 or 2, and especially 1 amino acid residue(s) may be altered) when compared to the reference sequence. In general, such conservative substitutions will fall within one of the amino-acid groupings specified below, though in some circumstances other substitutions may be possible without substantially affecting the immunogenic properties of the antigen. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

Suitably amino-acid substitutions are restricted to non-epitope regions of an antigen.

Polypeptide sequence variants may also include those wherein additional amino acids are inserted compared to the reference sequence, for example, such insertions may occur at 1-10 locations (such as 1-5 locations, suitably 1 or 2 locations, in particular 1) and may involve the addition of 50 or fewer amino acids (such as 20 or fewer, in particular 10 or fewer, especially 5 or fewer) at each location. Suitably such insertions do not occur in the region of an epitope, and do not therefore have a significant impact on the immunogenic properties of the antigen. One example of insertions includes a short stretch of histidine residues (e.g. 1-6 residues) to aid expression and/or purification of the antigen in question.

Other polypeptide sequence variants include those wherein amino acids have been deleted compared to the reference sequence, for example, such deletions may occur at 1-10 locations (such as 1-5 locations, suitably 1 or 2 locations, in particular 1) and may, for example, involve the deletion of 50 or fewer amino acids (such as 20 or fewer, in particular 10 or fewer, especially 5 or fewer) at each location. Suitably such deletions do not occur in the region of an epitope, and do not therefore have a significant impact on the immunogenic properties of the antigen.

Methods of determining the epitope regions of an antigen are described and exemplified elsewhere herein.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” or “hybridizes under stringent conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ, is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to fusion proteins can be selected to obtain only those polyclonal antibodies that are specifically immunoreactive with fusion protein and not with individual components of the fusion proteins. This selection may be achieved by subtracting out antibodies that cross-react with the individual antigens. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

Polynucleotides may comprise a native sequence (i.e., an endogenous sequence that encodes an individual antigen or a portion thereof) or may comprise a variant of such a sequence. Polynucleotide variants may contain one or more substitutions, additions, deletions and/or insertions such that the biological activity of the encoded fusion polypeptide is not diminished, relative to a fusion polypeptide comprising native antigens. Variants preferably exhibit at least about 70% identity, more preferably at least about 80% identity and most preferably at least about 90% identity to a polynucleotide sequence that encodes a native polypeptide or a portion thereof.

The terms “identical” or percent “identity,” in the context of two or more nucleic acids or polypeptide sequences, refer to two or more sequences or subsequences that are the same or have a specified percentage of amino acid residues or nucleotides that are the same (i.e., 70% identity, optionally 75%, 80%, 85%, 90%, or 95% (e.g. 98%) identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 25 to about 50 amino acids or nucleotides in length, or optionally over a region that is 75-100 amino acids or nucleotides in length. Suitably the identity exists over the entire length of the reference sequence. Variant polynucleotide and polypeptide sequences having at least 70% identity, optionally 75%, 80%, 85%, 90%, or 95% (e.g. 98%) identity over a specified region of a reference sequence (e.g. the whole length) are of particular interest.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 25 to 500, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted by, for example, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### Polynucleotide Compositions

As used herein, the terms “DNA segment” and “polynucleotide” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the DNA segments of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

The terms “isolated,” “purified,” or “biologically pure” therefore refer to material that is substantially or essentially free from components that normally accompany it as found in its native state. Of course, this refers to the DNA segment as originally isolated, and does not exclude other isolated proteins, genes, or coding regions later added to the composition by the hand of man. Purity and homogeneity are typically determined using analytical chemistry techniques such as polyacrylamide gel electrophoresis or high performance liquid chromatography. A protein that is the predominant species present in a preparation is substantially purified. An isolated nucleic acid is separated from other open reading frames that flank the gene and encode proteins other than the gene.

As will be recognised by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a Chlamydia antigen or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished. The effect on the immunogenicity of the encoded polypeptide may generally be assessed as described herein. The term “variants” also encompasses homologous genes of xenogenic origin.

In additional embodiments, the present invention utilises isolated polynucleotides and polypeptides comprising various lengths of contiguous stretches of sequence identical to or complementary to one or more of the sequences disclosed herein. For example, polynucleotides that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated, for example polynucleotides that are optimized for human and/or primate codon selection. Further, alleles of the genes comprising the polynucleotide sequences provided herein are also of use. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

### Polynucleotide Identification and Characterization

Polynucleotides may be identified, prepared and/or manipulated using any of a variety of well-established techniques. For example, a polynucleotide may be identified, as described in more detail below, by screening a microarray of cDNAs. Such screens may be performed, for example, using a Synteni microarray (Palo Alto, Calif.) according to the manufacturer's instructions (and essentially as described by Schena et al., Proc. Natl. Acad. Sci. USA 93:10614-10619 (1996) and Heller et al., Proc. Natl. Acad. Sci. USA 94:2150-2155 (1997)). Alternatively, polynucleotides may be amplified from cDNA prepared from cells expressing the proteins described herein, such as C. trachomatis cells. Such polynucleotides may be amplified via polymerase chain reaction (PCR). For this approach, sequence-specific primers may be designed based on the sequences provided herein, and may be purchased or synthesized.

An amplified portion of a polynucleotide may be used to isolate a full-length gene from a suitable library (e.g., a C. trachomatis cDNA library) using well-known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labelled (e.g., by nick-translation or end-labelling with ³²P) using well-known techniques. A bacterial or bacteriophage library is then generally screened by hybridizing filters containing denatured bacterial colonies (or lawns containing phage plaques) with the labelled probe (see Sambrook et al., Molecular Cloning: A Laboratory Manual (1989)). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full-length cDNA molecule can be generated by ligating suitable fragments, using well-known techniques.

Alternatively, there are numerous amplification techniques for obtaining a full-length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186 (1988)), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19 (1991)) and walking PCR (Parker et al., Nucl. Acids. Res. 19:3055-60 (1991)). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

### Polynucleotide Expression in Host Cells

Polynucleotide sequences or fragments thereof which encode polypeptides, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life that is longer than that of a transcript generated from the naturally occurring sequence.

Moreover, the polynucleotide sequences can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

Natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

In order to express a desired polypeptide, the nucleotide sequences encoding the polypeptide, or functional equivalents, may be inserted into appropriate expression vector, i.e., a vector that contains the necessary elements for the transcription and translation of the inserted coding sequence. Methods that are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke &Schuster, J. Biol. Chem. 264:5503-5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. Other vectors containing constitutive or inducible promoters include GAP, PGK, GAL and ADH. For reviews, see Ausubel et al. (supra), Grant et al., Methods Enzymol. 153:516-544 (1987) and Romas et al. Yeast 8 423-88 (1992).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill Yearbook of Science and Technology pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available.

For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus that is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines that stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-32 (1977)) and adenine phosphoribosyltransferase (Lowy et al., Cell 22:817-23 (1990)) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). Recently, the use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol. 55:121-131 (1995)).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques that include membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences that direct secretion of the encoded polypeptide through a prokaryotic or eukaryotic cell membrane. Other recombinant constructions may be used to join sequences encoding a polypeptide of interest to nucleotide sequence encoding a polypeptide domain that will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath et al., Prot. Exp. Purif. 3:263-281 (1992) while the enterokinase cleavage site provides a means for purifying the desired polypeptide from the fusion protein. A discussion of vectors which contain fusion proteins is provided in Kroll et al., DNA Cell Biol. 12:441-453 (1993)).

### In Vivo Polynucleotide Delivery Techniques

In additional embodiments, genetic constructs comprising polynucleotides are introduced into cells in vivo. This may be achieved using any of a variety or well-known approaches, several of which are outlined below for the purpose of illustration.

1. Adenovirus

One of the preferred methods for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized.

The expression vector comprises a genetically engineered form of an adenovirus. Knowledge of the genetic organization of adenovirus, a 36 kb, linear, double-stranded DNA virus, allows substitution of large pieces of adenoviral DNA with foreign sequences up to 7 kb (Grunhaus & Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the currently preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain a conditional replication-defective adenovirus vector for use in the present invention, since Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus & Horwitz, 1992; Graham & Prevec, 1992). Recently, animal studies suggested that recombinant adenovirus could be used for gene therapy (Stratford-Perricaudet & Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993).

2. Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding one or more oligonucleotide or polynucleotide sequences of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas & Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

3. Adeno-Associated Viruses

AAV (Ridgeway, 1988; Hermonat & Muzycska, 1984) is a parovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka & McLaughlin, 1988).

The AAV DNA is approximately 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p19 results in production of rep proteins, and transcription from p40 produces the capsid proteins (Hermonat & Muzyczka, 1984).

There are several factors that prompted researchers to study the possibility of using rAAV as an expression vector. One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering antisense constructs.

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response.

4. Other Viral Vectors as Expression Constructs

Other viral vectors may be employed as expression constructs in the present invention for the delivery of oligonucleotide or polynucleotide sequences to a host cell. Vectors derived from viruses such as vaccinia virus (Ridgeway, 1988; Coupar et al., 1988), lentiviruses, polioviruses and herpes viruses may be employed. Other poxvirus derived vectors, such as fowl-pox derived vectors, may also be expected to be of use. They offer several attractive features for various mammalian cells (Friedmann, 1989; Ridgeway, 1988; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. (1991) introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Additional ‘viral’ vectors include virus like particles (VLPs) and phages.

5. Non-Viral Vectors

In order to effect expression of the oligonucleotide or polynucleotide sequences n, the expression construct must be delivered into a cell. This delivery may be accomplished in vitro, as in laboratory procedures for transforming cells lines, or in vivo or ex vivo, as in the treatment of certain disease states. As described above, one preferred mechanism for delivery is via viral infection where the expression construct is encapsulated in an infectious viral particle.

Once the expression construct has been delivered into the cell the nucleic acid encoding the desired oligonucleotide or polynucleotide sequences may be positioned and expressed at different sites. In certain embodiments, the nucleic acid encoding the construct may be stably integrated into the genome of the cell. This integration may be in the specific location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In certain embodiments, the expression construct comprising one or more oligonucleotide or polynucleotide sequences may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty & Reshef (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated.

### Polypeptide Compositions

Generally, a polypeptide composition will be a combination of isolated polypeptides or immunogenic fragments thereof. Alternatively, some or all of the polypeptide antigens in an inventive composition may be within a fusion protein. For example, in an inventive composition comprising three antigens: (i) the antigens may be provided in the form of three isolated polypeptides (ii) all three polypeptides antigens may be provided in a single fusion protein (iii) two of the antigens may be provided in a fusion protein, with the third provided in isolated form. The proteins/polypeptides of the combination may be encoded by a polynucleotide sequence or sequences disclosed herein or a sequence or sequences that hybridize under moderately stringent conditions to a polynucleotide sequence or sequences disclosed herein. Alternatively, the proteins/polypeptides may be defined as polypeptides each comprising a contiguous amino acid sequence from an amino acid sequence disclosed herein (i.e. an immunogenic fragment of a sequence disclosed herein), or which proteins/polypeptides each comprise an entire amino acid sequence disclosed herein.

Immunogenic portions may generally be identified using well-known techniques, such as those summarized in Paul, Fundamental Immunology, 3rd ed., 243-247 (1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques. An immunogenic portion of a Chlamydia sp. protein is a portion that reacts with such antisera and/or T-cells at a level that is not substantially less than the reactivity of the full-length polypeptide (e.g., in an ELISA and/or T-cell reactivity assay). Such immunogenic portions may react within such assays at a level that is similar to or greater than the reactivity of the full-length polypeptide. Such screens may generally be performed using methods well known to those of ordinary skill in the art, such as those described in Harlow & Lane, Antibodies: A Laboratory Manual (1988). For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

Polypeptides may be prepared using any of a variety of well-known techniques. Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast or a mammalian cell line such as COS or CHO. Supernatants from suitable host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Polypeptides, immunogenic fragments thereof which may have for example less than about 100 amino acids, or less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146 (1963). Equipment for automated synthesis of polypeptides is commercially available from suppliers such as Perkin Elmer/Applied BioSystems Division (Foster City, Calif.), and may be operated according to the manufacturer's instructions.

Within certain specific embodiments, a polypeptide may be a fusion protein that comprises multiple polypeptides as described herein, or that comprises at least one polypeptide as described herein and an unrelated sequence, such as a known protein. Such a fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Thus, a fusion protein may be expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides. Typically fusion proteins comprising two or more antigens may omit the initiation codon (Met) from the second and subsequent antigens.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46 (1985); Murphy et al., Proc. Natl. Acad. Sci. USA 83:8258-8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Thus the compositions according to the invention may comprise one or more fusion proteins. Such proteins comprise a polypeptide component of the composition as described herein together with an unrelated immunogenic protein. The immunogenic protein may for example be capable of eliciting a recall response. Examples of such proteins include tetanus, tuberculosis and hepatitis proteins (see, e.g., Stoute et al., New Engl. J. Med. 336:86-91 (1997)).

Within certain embodiments, an immunological fusion partner is derived from protein D, a surface protein of the gram-negative bacterium Haemophilus influenza B (WO 91/18926). A protein D derivative may comprise approximately the first third of the protein (e.g., the first N-terminal 100-110 amino acids), and a protein D derivative may be lipidated. Within certain embodiments, the first 109 residues of a lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292 (1986)). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798 (1992)). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### T Cells

Immunotherapeutic compositions may also, or alternatively, comprise T cells specific for a Chlamydia antigen. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding such a polypeptide, and/or an antigen presenting cell (APC) that expresses such a polypeptide. Such stimulation is performed under conditions and for a time sufficient to permit the generation of T cells that are specific for the polypeptide. Preferably, the polypeptide or polynucleotide is present within a delivery vehicle, such as a microsphere, to facilitate the generation of specific T cells. T cells are considered to be specific for a polypeptide if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070 (1994)). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a polypeptide (100 ng/ml-100 μg/ml, preferably 200 ng/ml-25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1 (1998)). T cells that have been activated in response to a polypeptide, polynucleotide or polypeptide-expressing APC may be CD4⁺ and/or CD8⁺. Protein-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺T cells that proliferate in response to a polypeptide, polynucleotide or APC can be expanded in number either in vitro or in vivo. Proliferation of such T cells in vitro may be accomplished in a variety of ways. For example, the T cells can be re-exposed to a polypeptide, or a short peptide corresponding to an immunogenic portion of such a polypeptide, with or without the addition of T cell growth factors, such as interleukin-2, and/or stimulator cells that synthesize the polypeptide. Alternatively, one or more T cells that proliferate in the presence of the protein can be expanded in number by cloning. Methods for cloning cells are well known in the art, and include limiting dilution.

### Pharmaceutical Compositions

In additional embodiments, the polynucleotide, polypeptide, T-cell and/or antibody compositions disclosed herein will be formulated in pharmaceutically-acceptable or physiologically-acceptable solutions for administration to a cell or an animal, either alone, or in combination with one or more other modalities of therapy.

It will also be understood that, if desired, the nucleic acid segments, RNA, DNA or PNA compositions that express a composition of polypeptides as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation. Other routes of administration include via the mucosal surfaces, for example intravaginal administration.

1. Oral Delivery

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. No. 5,641,515; U.S. Pat. No. 5,580,579 and U.S. Pat. No. 5,792,451, each specifically incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

2. Injectable Delivery

In certain circumstances it will be desirable to deliver the pharmaceutical compositions disclosed herein parenterally, intravenously, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety). Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active components in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

3. Mucosal Delivery

(i) Nasal Delivery

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

(ii) Intravaginal Delivery

In other embodiments of the invention the pharmaceutical compositions may be formulated for intravaginal delivery. Such formulations may be prepared as liquids, semi-solids or solids (including for example, creams, ointments, gels etc), or may be contained within a physical delivery system such as a pessary, sponge, vaginal ring or film.

(iii) Ocular Delivery

In further embodiments of the invention the pharmaceutical compositions may be formulated for ocular delivery. Such formulations will desirably be clear and colourless.

(iv) Rectal Delivery

In additional embodiments of the invention the pharmaceutical compositions may be formulated for rectal delivery.

5. Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the nucleic acids or constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath & Martin, 1986; Heath et al., 1986; Balazsovits et al., 1989; Fresta & Puglisi, 1996), radiotherapeutic agents (Pikul et al., 1987), enzymes (Imaizumi et al., 1990a; Imaizumi et al., 1990b), viruses (Faller & Baltimore, 1984), transcription factors and allosteric effectors (Nicolau & Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mon & Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 urn. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety).

### Vaccines

In certain preferred embodiments of the present invention, vaccines are provided. The vaccines will generally comprise one or more pharmaceutical compositions, such as those discussed above, in combination with an immunostimulant. An immunostimulant may be any substance that enhances or potentiates an immune response (including antibody and/or cell-mediated) to an exogenous antigen. Examples of immunostimulants include adjuvants, biodegradable microspheres (e.g., polylactic galactide) and liposomes (into which the compound is incorporated; see, e.g., Fullerton, U.S. Pat. No. 4,235,877). Vaccine preparation is generally described in, for example, Powell & Newman, eds., Vaccine Design (the subunit and adjuvant approach) (1995). Pharmaceutical compositions and vaccines within the scope of the present invention may also contain other compounds, which may be biologically active or inactive. For example, one or more immunogenic portions of other antigens may be present, either incorporated into a fusion polypeptide or as a separate compound, within the composition or vaccine.

Illustrative vaccines may contain DNA encoding two or more of the polypeptides as described above, such that the polypeptides are generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198 (1998), and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321 (1989); Flexner et al., Ann. N.Y. Acad. Sci. 569:86-103 (1989); Flexner et al., Vaccine 8:17-21 (1990); U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627 (1988); Rosenfeld et al., Science 252:431-434 (1991); Kolls et al., Proc. Natl. Acad. Sci. USA 91:215-219 (1994); Kass-Eisler et al., Proc. Natl. Acad. Sci. USA 90:11498-11502 (1993); Guzman et al., Circulation 88:2838-2848 (1993); and Guzman et al., Cir. Res. 73:1202-1207 (1993). Techniques for incorporating DNA into such expression systems are well known to those of ordinary skill in the art. The DNA may also be “naked,” as described, for example, in Ulmer et al., Science 259:1745-1749 (1993) and reviewed by Cohen, Science 259:1691-1692 (1993). The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

While any suitable carrier known to those of ordinary skill in the art may be employed in the vaccine compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. One may also employ a carrier comprising the particulate-protein complexes described in U.S. Pat. No. 5,928,647, which are capable of inducing a class I-restricted cytotoxic T lymphocyte responses in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium species or Mycobacterium derived proteins. For example, delipidated, deglycolipidated M. vaccae (“pVac”) can be used. In another embodiment, BCG is used as an adjuvant. In addition, the vaccine can be administered to a subject previously exposed to BCG. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition may be designed to induce an immune response predominantly of the Th1 type. High levels of Th1-type cytokines (e.g., IFN-γ, TNFα, IL-2 and IL-12) tend to favor the induction of cell-mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within one embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann & Coffman, Ann. Rev. Immunol. 7:145-173 (1989).

Suitable adjuvants for use in eliciting a predominantly Th1-type response include, for example, a combination of monophosphoryl lipid A, for example 3-de-O-acylated monophosphoryl lipid A (3D-MPL), together with an aluminum salt. MPL adjuvants are available from Corixa Corporation (now part of GlaxoSmithKline; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another suitable adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other suitable formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol^{R} to increase viscosity, or may be formulated in a dry powder form with a powder excipient such as lactose.

In one embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol containing liposomes, as described in WO 96/33739. Other suitable formulations comprise an oil-in-water emulsion and tocopherol. Another suitable adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 as disclosed in WO 00/09159. Suitably the formulation additionally comprises an oil in water emulsion and tocopherol.

Other suitable adjuvants include Montanide ISA 720 (Seppic, France), SAF (Chiron, Calif., United States), ISCOMS (CSL), MF-59 (Chiron), the SBAS series of adjuvants (SmithKline Beecham, Rixensart, Belgium), Detox (Corixa), RC-529 (Corixa) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1. SmithKline Beecham and Corixa Corporation are now part of GlaxoSmithKline.

Other suitable adjuvants include adjuvant molecules of the general formula (I):

HO(CH₂CH₂O)ₙ-A-R

wherein, n is 1-50, A is a bond or —C(O)—, R is C₁₋₅₀ alkyl or Phenyl C₁₋₅₀ alkyl.

A further adjuvant of interest is shiga toxin b chain, used for example as described in WO2005/112991.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438 (1996)) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see, e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain embodiments of the present invention use dendritic cells or progenitors thereof as antigen-presenting cells. Dendritic cells are highly potent APCs (Banchereau & Steinman, Nature 392:245-251 (1998)) and have been shown to be effective as a physiological adjuvant for eliciting prophylactic or therapeutic antitumor immunity (see Timmerman & Levy, Ann. Rev. Med. 50:507-529 (1999)). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600 (1998)).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allow a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1 BB).

APCs may generally be transfected with a polynucleotide encoding a protein (or portion or other variant thereof) such that the polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and Cell Biology 75:456-460 (1997). Antigen loading of dendritic cells may be achieved by incubating dendritic cells or progenitor cells with the polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

### EXAMPLES

The following examples are provided by way of illustration only and do not serve to limit the scope of the invention. Those skilled in the art will recognise that a variety of non-critical parameters are described which could be adapted to yield similar results.

### Example 1

### Ct-089, Ct-858 and Ct-875 Sequence Comparisons

Chlamydia trachomatis serovar E is a common oculogenital serovar and was chosen as a basis to which the other sequences would be compared.

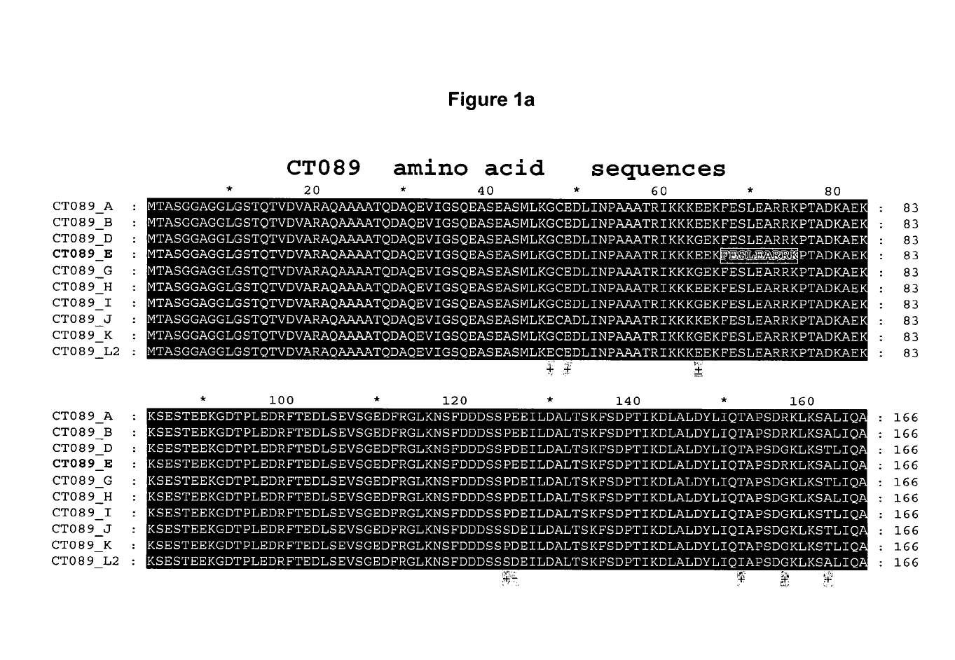

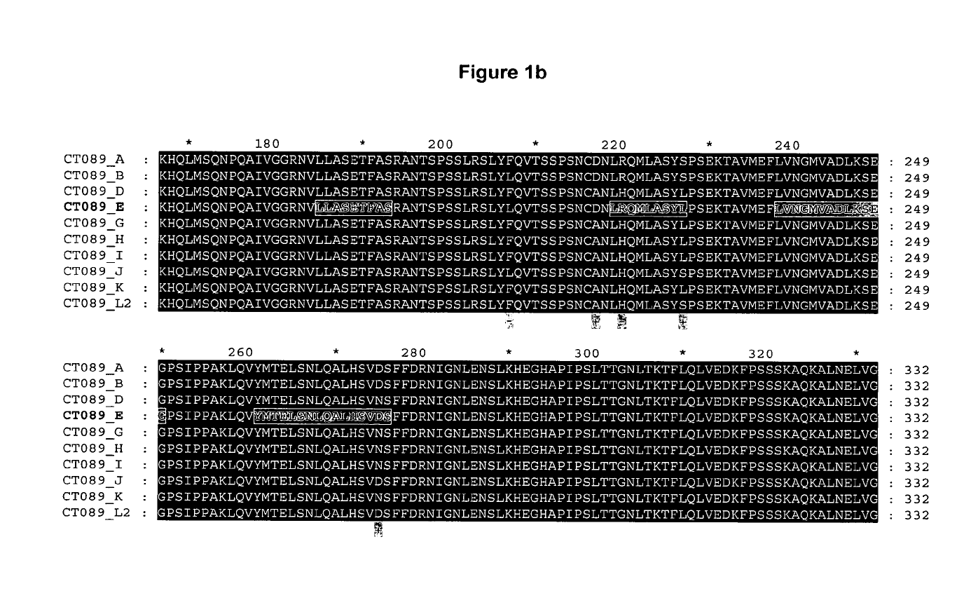

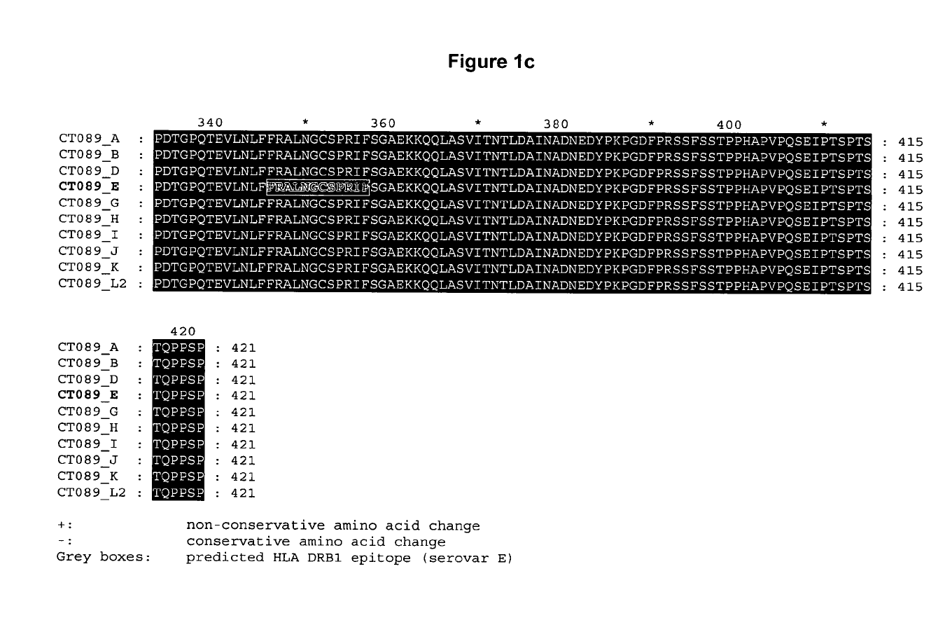

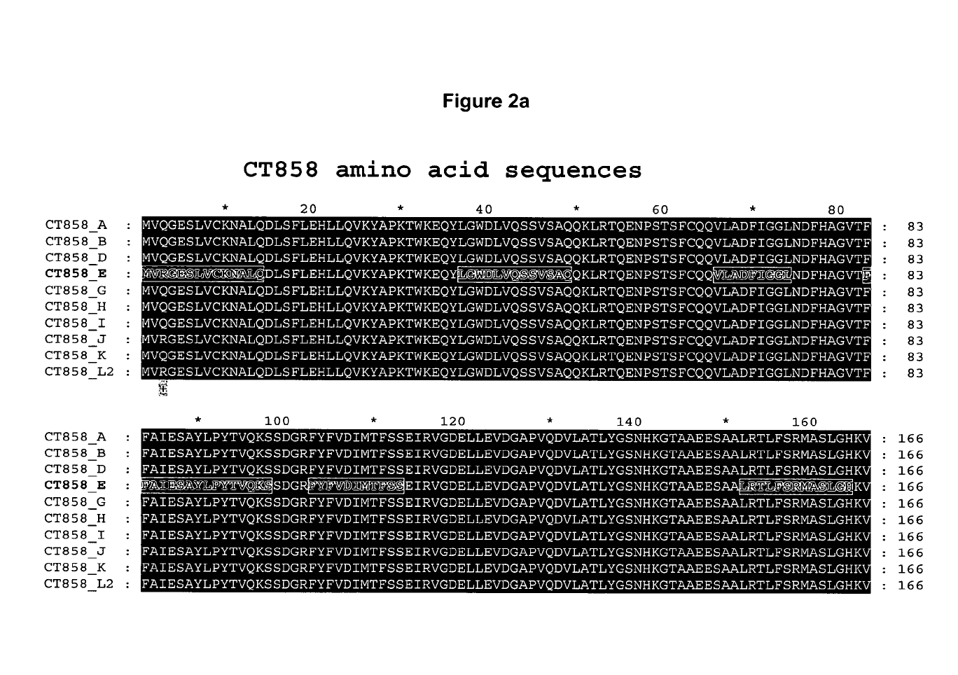

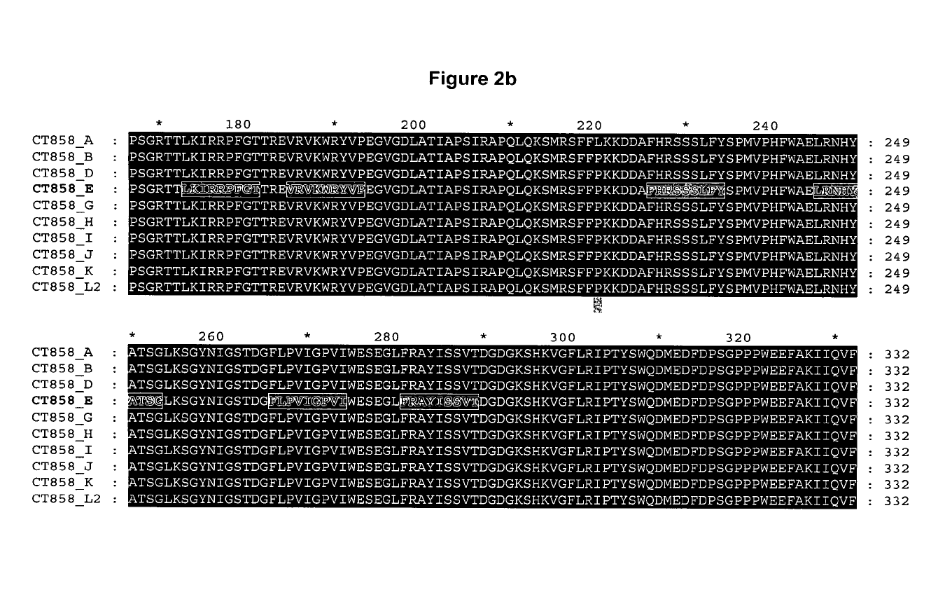

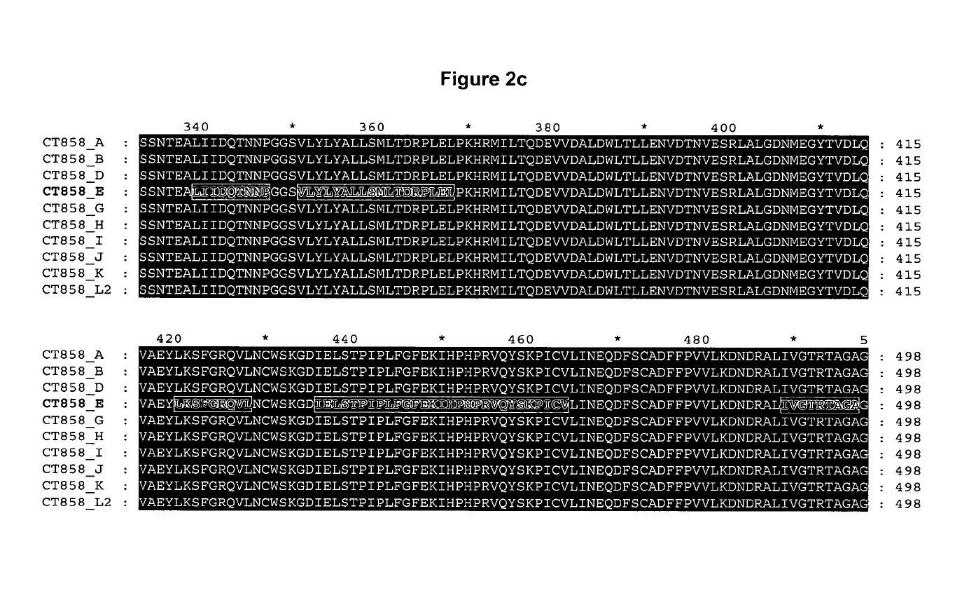

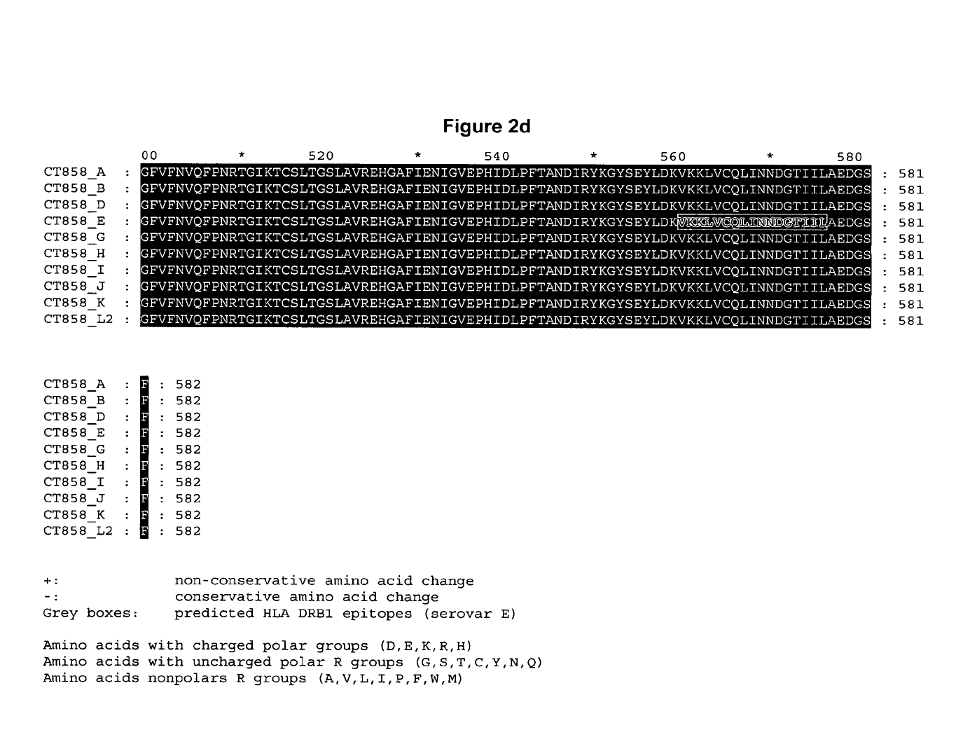

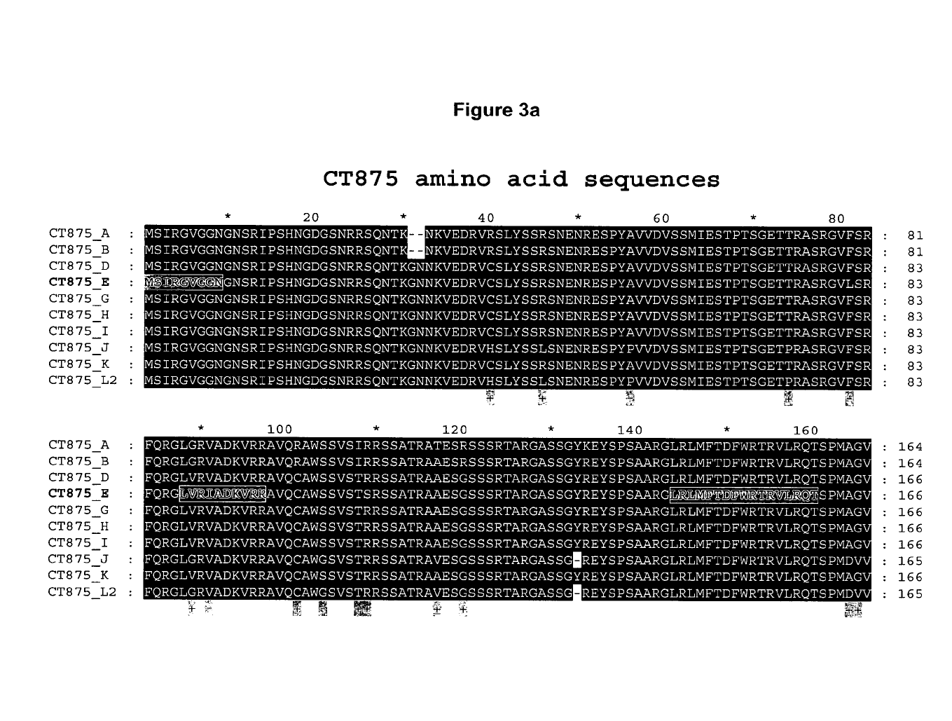

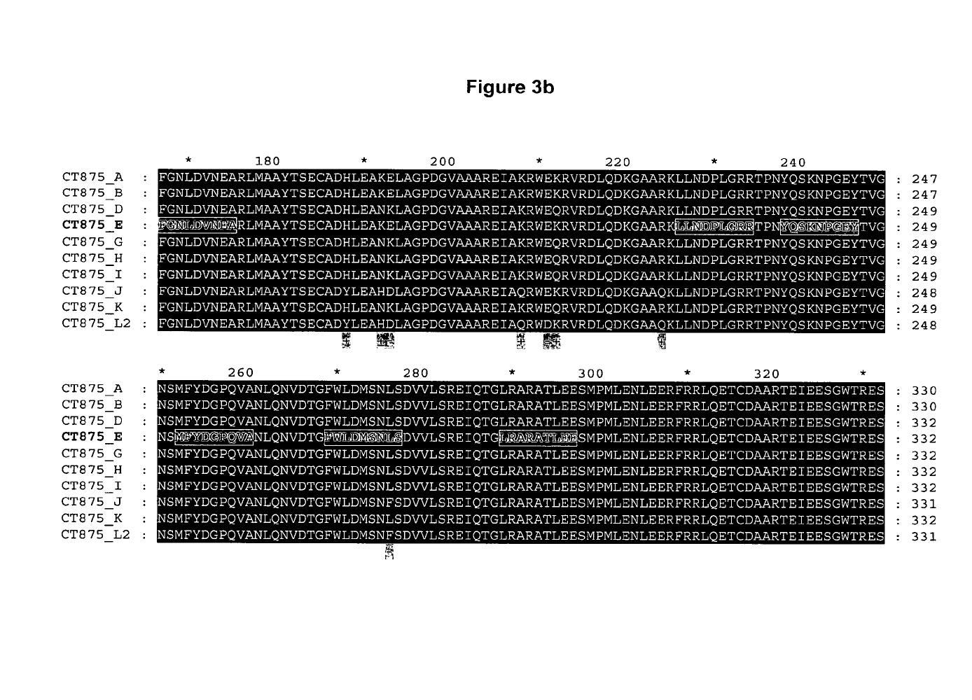

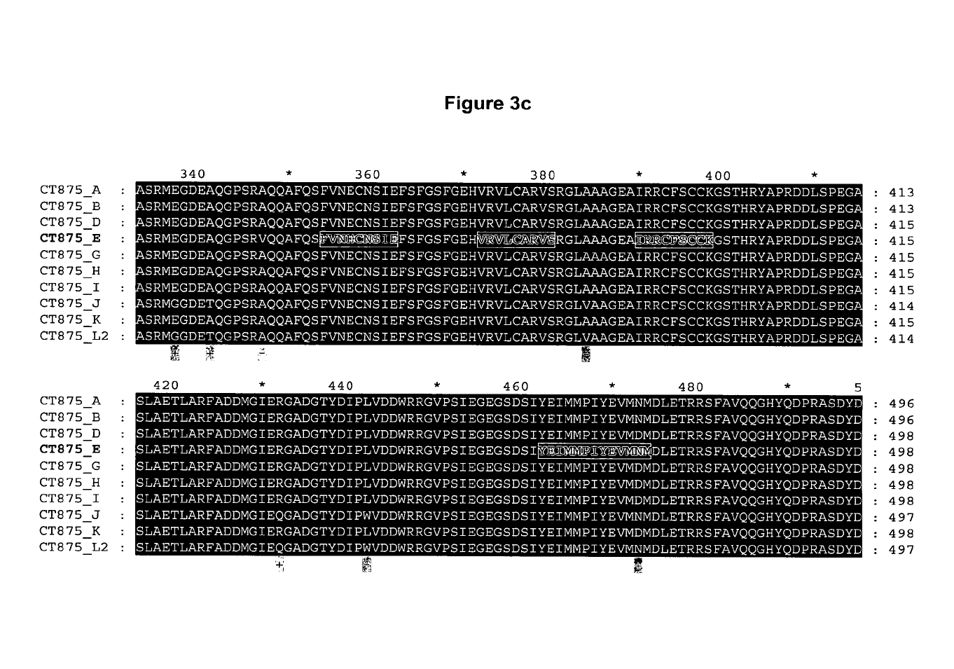

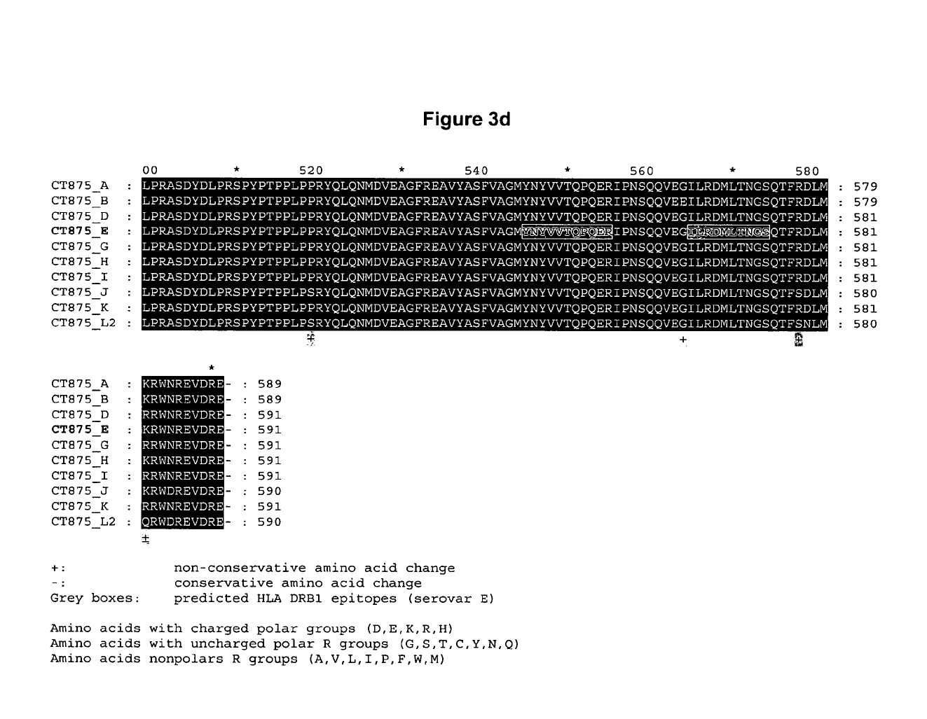

A multiple alignment of amino-acid sequences for comparison has been conducted using the CLUSTAL W program, available in the Lasergene software package, version 5.0 (sold by DNASTAR, Inc., Madison, Wis.)). The basic multiple alignment algorithm involves a three-step procedure: all pairs of sequences are aligned separately in order to calculate a distance matrix giving the divergence of each pair of sequences, then a guide tree is calculated from the distance matrix and finally the sequences are progressively aligned according to the guide tree. CLUSTAL W algorithm is described in Thompson et al., Nuc. Acids Res. 22: 4673-4680 (1994). The alignments are shown in FIGS. 1, 2a/2b and 3a/3b.

The T-helper cell epitopes are peptides bound to HLA class II molecules and recognized by T-helper cells. The prediction of putative T-helper cell epitopes, present on Ct-089, Ct-858 and Ct-875 Chlamydia trachomatis polypeptides from serovar E, was based on the TEPITOPE method described by Sturniolo et al., Nature Biotech. 17:555-561 (1999). The peptides comprising good, potential T-cell epitopes are highlighted (grey boxes) in FIGS. 1, 2a/2b and 3a/3b.

### Example 2

### Eliciting a Protective Immune Response Against Ocular Chlamydia trachomatis Infection in Mice

### Experiment Summary

Female C57BL/6 and C3H mice were vaccinated (two or three intramuscular immunisations, with two different dosage levels) using a combination of Ct-089, Ct-858 and Ct-875 proteins from serovar E formulated in adjuvant. A positive control group was vaccinated using UV attenuated elementary bodies from serovar A or K in adjuvant. A negative control group was vaccinated using adjuvant only.

Mice were infected by a single ocular challenge with ocular serovars A, B or oculogenital serovar K. The course of infection was monitored by performing ocular swabs.

### Method

### Test Subjects

Two hundred and forty, six week old female mice (consisting of one hundred and forty four C3H mice and ninety six C57BL/6 mice) were obtained from Charles River Laboratories (Wilmington, Mass.). Animals were divided into thirty groups of eight mice each (eighteen groups of C3H mice and twelve groups of C57BL/6 mice). Six experimental groups of C3H mice were used for challenge with each of serovars A, B or K. Six experimental groups of C57BL/6 mice were used for challenge with each of serovars A or K.

Four groups of mice in each subset were immunised according to the present invention (two or three immunizations, at low or high dosage). The remaining two groups in each subset were used for controls with UVEB in adjuvant or adjuvant alone.

Each group of mice was caged individually and housed under a 12 hour dark/12 hour light cycle.

### Bacteria Preparation

### Live Elementary Bodies (EB)

The Chlamydia trachomatis serovars A, B and K were obtained from the American Type Culture Collection (ATCC) and expanded before use in the challenge of mice. The original stock titres were 1.2×10⁷ IFU/ml for serovar K, 1.4×10⁷ IFU/ml for serovar B and 1.92×10⁹ IFU/ml for serovar A.

The stock serovars were raised in McCoy cells in 75 cm² culture flasks. Confluent cell monolayers in culture flasks were inoculated with the respective serovar, spun at 2000 rpm for one hour and incubated for 48 hours at 37° C. with 5% CO₂ in RPMI 1640 supplemented with 10% fetal bovine serum, 1 mM sodium pyruvate, 1×MEM NEA acids, 50 uM Bme β-mercaptoethanol, 10 mg/L of mycostatin and 10 mg/L of vancomycin. Cyclohexamide at 1 ug/ml was added before infection (cyclohexamide is a protein synthesis inhibitor which favours Chlamydial replication in order to establish infection). Chlamydial elementary bodies (EB) were harvested post infection by disruption of cell monolayers with 5 mm of glass beads and frozen in SPG at −80° C. To obtain high titres, serovars were cultured for at least four cycles on McCoy cells monolayers in culture flasks. Semi-purification was not performed unless 90 to 100% of the cells were infected in each culture flask upon examination under light microscopy.

Viable elementary bodies from at least twenty 75 cm² infected culture flasks were semi-purified over an initial 30% Hypaque gradient and secondarily on 52%, 44% and 40% Hypaque gradients, using ultracentrifugation for the gradients. The final pellet after two washes was resuspended in SPG (75 g sucrose, 0.52 g potassium phosphate, 2.3 g sodium phosphate dibasic heptahydrate and 0.72 g glutamic acid, pH 7.5, sterile) in cryovials and frozen at −80° C. for later use.

### UV-Attenuated Elementary Bodies (UVEB)

For the purposes of control immunisations, purified serovar A and K elementary bodies were inactivated under UV light. Thin layers of EB suspensions were placed in a six well plate directly under a UV lamp (Sanyo germicidal lamp) 1 inch from the light, for a period of 1 hour. The UVEBs were standardized according to protein content determined by BCA protein assay, aliquoted and frozen. The concentration of the stock UVEB for serovar A was 249.3 ug/ml and for serovar K was 5145 ug/ml.

A viability test for the UVEB was performed in McCoy cell monolayers.

### Vaccine Preparation

### Adjuvant Control

The adjuvant utilised was based upon a liposomal formulation containing 3D-MPL, QS21 and cholesterol. The final composition of the adjuvant solution was:

| | | |
|---|---|---|
| 3D-MPL | 100 | ug/ml |
| QS21 | 100 | ug/ml |
| DOPC | 2 | mg/ml |
| Cholesterol | 0.5 | mg/ml |

(DOPC = dioleoylphosphatidylcholine)

Phosphate buffered saline was prepared from 9 mM Na₂HPO₄, 48 mM KH₂PO₄ and 100 mM NaCl at pH 6.1.

A mixture of lipid, cholesterol and 3D-MPL was prepared in organic solvent, this was then dried under vacuum. PBS was then added and the vessel agitated until a suspension formed. This suspension was then microfluidised until a liposome size of around 100 nm was obtained (referred to as small unilamellar vesicles or SUV). Subsequently, the SUV were sterilized by passage through a 0.2 um filter.

Sterile SUV were mixed with the appropriate quantity of aqueous QS21 (at a concentration of 2 mg/ml) with the addition of phosphate buffered saline to obtain the final desired concentrations. The pH was then adjusted to 6.1 (+/−0.1) as necessary using sodium hydroxide or hydrochloric acid.

### UVEB in Adjuvant

10 ug of UVEB was formulated in a volume of 100 ul by the mixing of 50 ul of the required UVEB (i.e. stock UVEB concentration adjusted to 20 ug/ul) with 50 ul of double strength adjuvant.

### Ct-089, Ct-858 and Ct-875 Proteins in Adjuvant

Protein antigens were prepared using conventional means. Briefly, competent E. coli strains BL21 plys E, Tuner (DE3) and BL21 plys S were transformed with Ct-089, Ct-858 and Ct-875 expression plasmids respectively and grown on the appropriate antibiotic selection medium. The resulting expression clones were used in a mini-induction protocol, and protein yields analyzed by SDS-PAGE. If cells grew well during this process and proteins were induced by isopropyl-beta-D-thiogalactopyranoside (IPTG) in sufficient quantities to be detected on Coomassie blue-stained SDS gels, the clones were used in a large-scale induction experiment (IPTG, 1 mM). Following lysis of cells in a CHAPS solution and centrifugation, aliquots of the soluble and pellet fractions were analyzed by SDS-PAGE to determine whether the majority of the protein of interest was in the pellet or soluble fraction. The fraction containing the majority of each antigen was subjected to Ni-NTA column purification (after appropriate solubilisation of proteins). Aliquots of the preparations, including material from before Ni-NTA binding, column flow-through, column washes, and column elution fractions, were analyzed by SDS-PAGE. Fractions containing the eluted protein were combined, dialyzed against 10 mM Tris pH 8 or pH 10, filtered sterilized, and concentrated. The BCA protein assay was used on the concentrated Ct protein fractions, and purity was assessed by SDS-PAGE.

Two compositions containing Ct-089, Ct-858 and Ct-875 from Chlamydia trachomatis serovar E with adjuvant (as described above) were prepared. The first (lose dose) having 1.25 ug of each protein antigen in 100 ul of composition, the second (high dose) having 5 ug of each protein in 100 ul of composition.

### Immunisation and Challenge

### Anaesthetic

Prior to immunisations mice were anaesthetised by injectable anaesthetic (Ketaject-Xylaject 1:1 dose) with 30 ul given intraperitoneally per mouse.

Prior to ocular challenge and ocular swabs, mice were anaesthetised by injectable anaesthetic (Ketaject-Xylaject 1:1 dose) with 30 ul given intraperitoneally per mouse and 20 ul intramuscularly to each thigh.

### Immunisations

The immunisations were given once, twice or three times on days 0, 21 and 42 (as appropriate). Mice were injected intramuscularly using a total volume of 100 ul per mouse, injecting 50 ul of the formulation in each thigh.

Groups of mice receiving treatment according to the invention were intramuscularly immunised with the exemplary combination vaccine of three Chlamydia trachomatis proteins (Ct-089, Ct-858 and Ct-875, 1.25 ug of each for low dose, 5 ug of each for high dose) in 100 ul adjuvant formulation. Treatment mice were immunised with either two or three doses on days 0, 21 and also day 42 for those receiving three doses.

Positive control groups of mice were intramuscularly immunised with 10 ug of UVEB in 100 ul adjuvant formulation, receiving immunisations either once or three times on day 0 and also days 21 and 42 for those receiving three doses. The negative control groups were intramuscularly immunised with 100 ul adjuvant formulation, receiving immunisations three times on days 0, 21 and 42.

### Challenge

Freshly thawed Chlamydia trachomatis EB aliquots from serovar A, B or K were each separately diluted in cold SPG buffer to a final concentration of 5×10³ IFU in 5 ul. The inoculums were kept on ice during inoculations. Deeply anaesthetised mice were challenged on day 70 with 5×10³ IFU of the appropriate serovar, in 5 ul per eye, by topical application to the upper formix with a micropipette using new sterile pipette tips for each eye.

### Infection Monitoring

The course of infection following ocular exposure was monitored by performing ocular swabs on days 7, 14 and 21 following challenge and analysing the swabs from the presence of IFU.

At the end of the experiment terminal bleeding was performed by heart puncture under deep anaesthesia (obtaining up to 1 ml of blood each from each mouse). The samples were processed immediately and stored at −20°. Mice were then euthanized using CO₂.

### Swabs

The swabs (sterile polyester tipped applicators) were pre-wetted in 1 ml SPG in their respective cryovial. Each swab was rotated in the conjunctiva and eye lid 30 turns each area while each mouse was deeply anaesthetised. The swab was then placed in the respective cryovial and placed in dry ice. The cryovials containing the swabs were stored at −80° C.

Titration of swabs was performed with 24-well plates containing confluent monolayers of McCoy cells in medium with cyclohexamide (1 ug/ml). Once thawed, the cryovials containing the swabs were vortexed for 5 min in the presence of glass beads. 100 ul from each of the cryovials containing swabs was inoculated in one well on duplicate 24-well plates containing a McCoy cell monolayer in 1 ml medium with cyclohexamide. After centrifugation at 2000 rpm for 1 hr the plates were incubated at 37° C. and 5% CO₂. The monolayers were fixed in methanol at 48 hours after infection and stained by Evans Blue and FITC-conjugated anti-Chlamydia trachomatis antibody.

The monolayers were examined for inclusions by inverted fluorescence microscopy. The method used for calculating the number of IFU per swab consisted of counting the whole well under a fluorescence microscope and then multiplying by the dilution factor of 10. When no inclusion bodies were observed, an arbitrary value below the detection limit of 10 (usually 7) was used to represent the number of IFU/swab.

### ELISA

Enzyme-linked immunosorbent assay was performed on serum samples. Whole A or K EB separately diluted in 0.1 M phosphate-buffered saline (PBS) KPL Coating Solution Concentrate (pH 7.2 to 7.4) served as antigen (˜106 FU/well). Serial dilutions of serum (1:2) were done after blocking with PBS-0.05% Tween, 1% BSA, followed by sequential washes in PBS-0.05% Tween and the addition of alkaline-phosphatase conjugated secondary antibody to the wells IgG+IgM+IgA (Kirkegaard & Perry, Gaithersburg, Md.). Reactions were developed with nitrophenylphosphate in diethanolamine substrate buffer (KPL p-NPP microwell substrate system) and the absorbance in each well (OD₄₀₅) was taken after 30 to 60 min.

### Treatment Summary

| | | | Treatment | | | | | Challenge |
|---|---|---|---|---|---|---|---|---|
| | Subjects | | | | | | Inoculations | Serovar |
| Group | n | Strain | Antigen | Serovar | Amount | | (day) | (day 70) |
| 1 | 8 | C3H | — | — | — | | 3 (0, 21, 42) | K |
| 2 | 8 | C3H | UVEB | K | 10 | ug | 1 (0) | K |
| 3 | 8 | C3H | Ct-089, Ct-858, Ct-875 | E | 1.25 | ug | 2 (0, 21) | K |
| 4 | 8 | C3H | Ct-089, Ct-858, Ct-875 | E | 5 | ug | 2 (0, 21) | K |
| 5 | 8 | C3H | Ct-089, Ct-858, Ct-875 | E | 1.25 | ug | 3 (0, 21, 42) | K |
| 6 | 8 | C3H | Ct-089, Ct-858, Ct-875 | E | 5 | ug | 3 (0, 21, 42) | K |
| 7 | 8 | C57BL/6 | — | — | — | | 3 (0, 21, 42) | K |
| 8 | 8 | C57BL/6 | UVEB | K | 10 | ug | 3 (0, 21, 42) | K |
| 9 | 8 | C57BL/6 | Ct-089, Ct-858, Ct-875 | E | 1.25 | ug | 2 (0, 21) | K |
| 10 | 8 | C57BL/6 | Ct-089, Ct-858, Ct-875 | E | 5 | ug | 2 (0, 21) | K |
| 11 | 8 | C57BL/6 | Ct-089, Ct-858, Ct-875 | E | 1.25 | ug | 3 (0, 21, 42) | K |
| 12 | 8 | C57BL/6 | Ct-089, Ct-858, Ct-875 | E | 5 | ug | 3 (0, 21, 42) | K |
| 13 | 8 | C3H | — | — | — | | 3 (0, 21, 42) | A |
| 14 | 8 | C3H | UVEB | A | 10 | ug | 3 (0, 21, 42) | A |
| 15 | 8 | C3H | Ct-089, Ct-858, Ct-875 | E | 1.25 | ug | 2 (0, 21) | A |
| 16 | 8 | C3H | Ct-089, Ct-858, Ct-875 | E | 5 | ug | 2 (0, 21) | A |
| 17 | 8 | C3H | Ct-089, Ct-858, Ct-875 | E | 1.25 | ug | 3 (0, 21, 42) | A |
| 18 | 8 | C3H | Ct-089, Ct-858, Ct-875 | E | 5 | ug | 3 (0, 21, 42) | A |
| 19 | 8 | C57BL/6 | — | — | — | | 3 (0, 21, 42) | A |
| 20 | 8 | C57BL/6 | UVEB | A | 10 | ug | 3 (0, 21, 42) | A |
| 21 | 8 | C57BL/6 | Ct-089, Ct-858, Ct-875 | E | 1.25 | ug | 2 (0, 21) | A |
| 22 | 8 | C57BL/6 | Ct-089, Ct-858, Ct-875 | E | 5 | ug | 2 (0, 21) | A |
| 23 | 8 | C57BL/6 | Ct-089, Ct-858, Ct-875 | E | 1.25 | ug | 3 (0, 21, 42) | A |
| 24 | 8 | C57BL/6 | Ct-089, Ct-858, Ct-875 | E | 5 | ug | 3 (0, 21, 42) | A |
| 25 | 8 | C3H | — | — | — | | 3 (0, 21, 42) | B |
| 26 | 8 | C3H | UVEB | K | 10 | ug | 3 (0, 21, 42) | B |
| 27 | 8 | C3H | Ct-089, Ct-858, Ct-875 | E | 1.25 | ug | 2 (0, 21) | B |
| 28 | 8 | C3H | Ct-089, Ct-858, Ct-875 | E | 5 | ug | 2 (0, 21) | B |
| 29 | 8 | C3H | Ct-089, Ct-858, Ct-875 | E | 1.25 | ug | 3 (0, 21, 42) | B |
| 30 | 8 | C3H | Ct-089, Ct-858, Ct-875 | E | 5 | ug | 3 (0, 21, 42) | B |

### Results

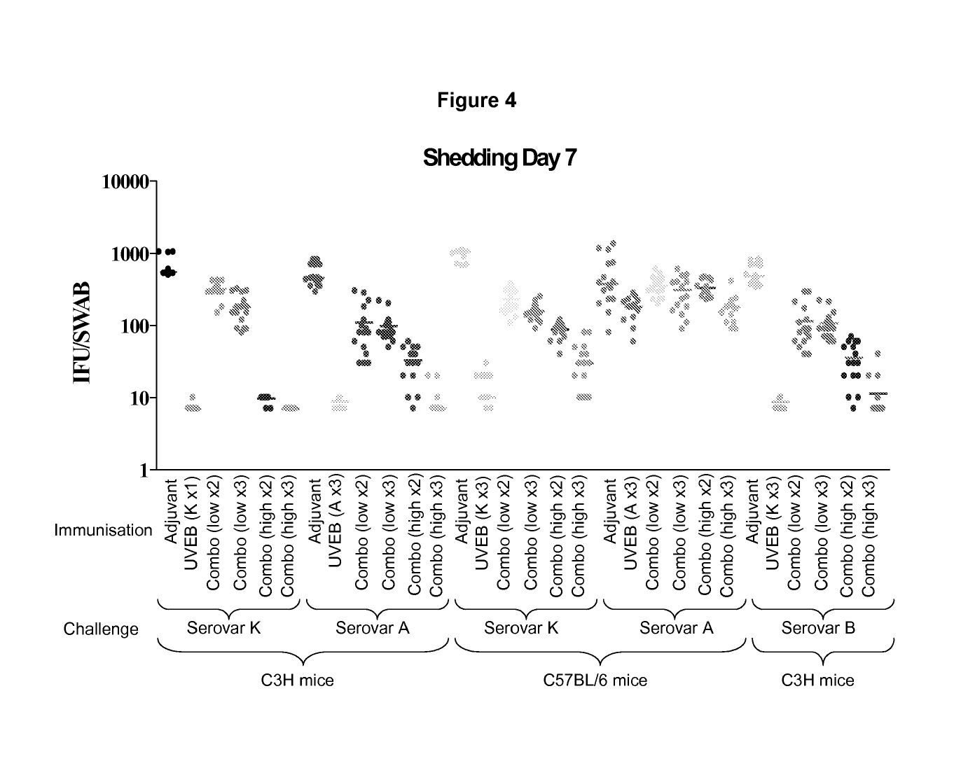

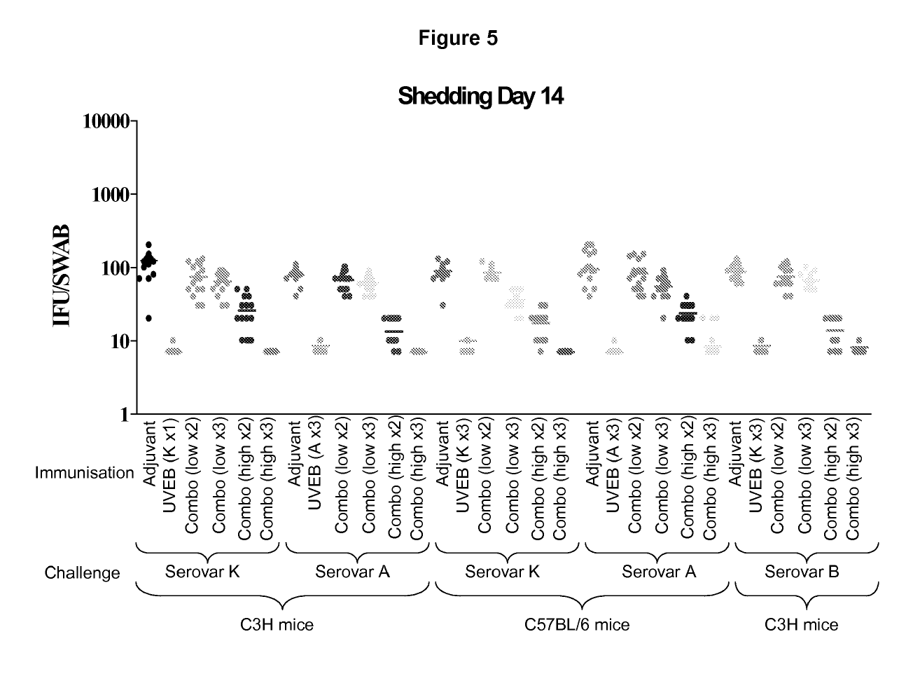

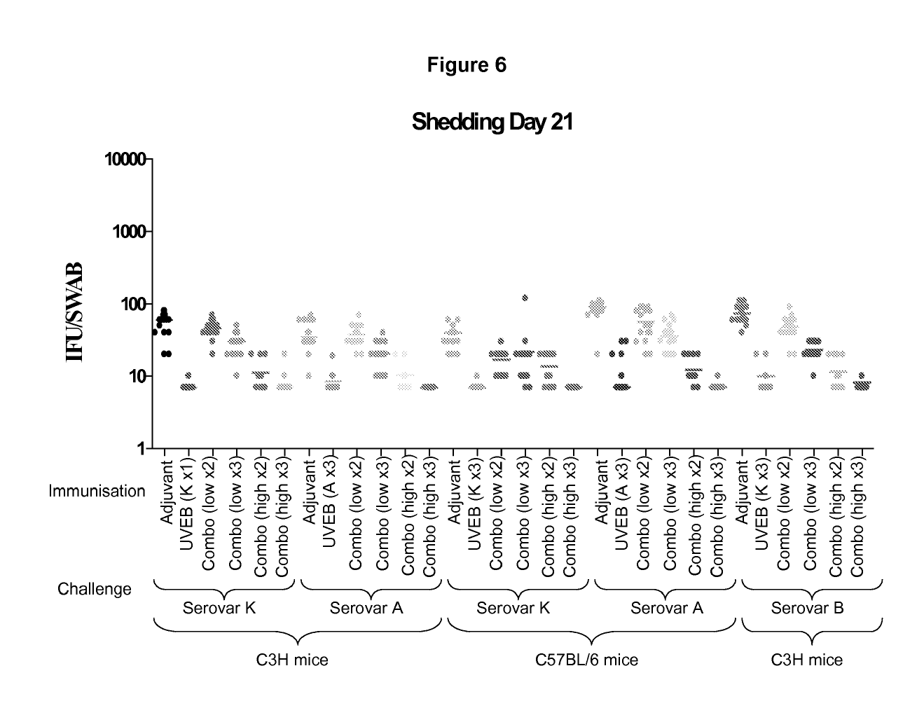

FIGS. 4 to 6 show the number of IFU present on ocular swabs taken respectively on days 7, 14 and 21 after challenge.

Statistical analysis of the data leads to the following key observations:

Comparison of Negative Control (Adjuvant Only) and Positive Control (UVEB in Adjuvant) Groups

Unpaired T tests show that UVEB A immunisation provides statistically significant protection compared to adjuvant only in C3H mice on days 7, 14 and 21 after challenge with serovar A (p<0.0001).

Unpaired T tests show that UVEB A immunisation provides statistically significant protection compared to adjuvant only in C57BL/6 mice on days 7, 14 and 21 after challenge with serovar A (p=0.0019 for day 7, p<0.0001 for days 14 and 21).

At days 7, 14 and 21, Anova-Dunnett's Multiple Comparison tests show UVEB A immunisation in both C3H and C57BL/6 groups provides statistically significant protection compared to adjuvant only after challenge with serovar A (p<0.01).

Unpaired T tests show that UVEB K immunisation provides statistically significant protection compared to adjuvant only in C3H mice on days 7, 14 and 21 after challenge with serovar K (p<0.0001).

Unpaired T tests show that UVEB K immunisation provides statistically significant protection compared to adjuvant only in C57BL/6 mice on days 7, 14 and 21 after challenge with serovar K (p<0.0001).

At day 7, 14 and 21, Anova-Dunnett's Multiple Comparison tests show UVEB K immunisation in both C3H and C57BL/6 groups provides statistically significant protection compared to adjuvant only after challenge with serovar K (p<0.01).

Comparison of Negative Control with Treatment Groups (i.e. ×3 Immunisations at High Dose)

Unpaired T tests comparing negative controls (i.e. adjuvant only) with immunisation according to the present invention using a combination of Ct-089, Ct-858 and Ct-875 proteins shows a significant difference in the protection conferred following challenge with serovar A or serovar K in both C3H and C57BL/6 mice at days 7, 14 and 21 (p<0.0001).

Anova-Tukey's Test comparing negative controls (i.e. adjuvant only) with immunisation according to the present invention using a combination of Ct-089, Ct-858 and Ct-875 proteins shows a significant difference in the protection conferred following challenge with serovar B in C3H mice at days 7, 14 and 21 (p<0.001).

At days 7, 14 and 21, Anova-Dunnett's Multiple Comparison tests show significant statistical differences in the protection conferred by the negative control when compared to the combination treatment in both C3H and C57BL/6 mice after challenge with serovar A (p<0.01).

Comparison of Positive Controls with Triple Immunised High Dose Treatment Groups

At days 7, 14 and 21, Anova-Dunnett's Multiple Comparison tests show no significant statistical differences (p>0.05) between the positive control (i.e. UVEB immunisation) and the corresponding combination treatment in C3H and C57BL/6 mice following challenge with serovar A.

At days 7, 14 and 21, Anova-Dunnett's Multiple Comparison tests show no significant statistical differences (p>0.05) between the positive control (i.e. UVEB immunisation) and the corresponding combination treatment in C3H and C57BL/6 mice following challenge with serovar K.

At days 7, 14 and 21, Anova-Dunnett's Multiple Comparison tests show no significant statistical differences (p>0.05) between the positive control (i.e. UVEB immunisation) and the corresponding combination treatment in C3H mice following challenge with serovar B.

### CONCLUSION

Adjuvant alone (negative control) is unable to confer protection against ocular infection.

UVEBs in adjuvant (positive control) from serovar A or K confer protection against ocular infection with serovars A and K respectively in both mice strains at all time points.

Treatment with immunogenic compositions according to the present invention (which are derived from serovar E in each case) results in statistically significant protection against ocular infection with either serovar A or serovar K in either mice strain at all time points when three high dose immunisations are provided. Similar levels of protection are observed in respect of serovar B challenge in C3H mice, although statistical analysis has not been performed to confirm the significance of this result.

Two high dose immunisations provide improved protection in all cases when compared to three low dose immunisations.

The results show that treatment according to the present invention provides substantial protection against ocular infection (equivalent to UVEB), it is capable of eliciting protection from serovars other than that from which the immunogenic composition is derived (i.e. cross-serovar protection from ocular infection). Furthermore, such protection may be achieved by administration via a non-ocular route.

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A method for the treatment or prevention of ocular Chlamydia trachomatis infection by the administration of an immunogenic composition comprising a Chlamydia trachomatis protein selected from the group consisting of Swib, Momp, Ct-858, Ct-875, Ct-622, Ct-089, passenger domain of PmpG (PmpGpd) and passenger domain of PmpD (PmpDpd).

2. An immunogenic composition comprising a Chlamydia trachomatis protein selected from the group consisting of Swib, Momp, Ct-858, Ct-875, Ct-622, Ct-089, passenger domain of PmpG (PmpGpd) and passenger domain of PmpD (PmpDpd).

3. (canceled)

4. The composition according to claim 2 wherein the immunogenic composition comprises two Chlamydia trachomatis proteins selected from the group consisting of Swib, Momp, Ct-858, Ct-875, Ct-622, Ct-089, passenger domain of PmpG (PmpGpd) and passenger domain of PmpD (PmpDpd).

5.-9. (canceled)

10. The composition according to claim 2, wherein the immunogenic composition comprises Ct-858 and Ct-875.

11. The composition according to claim 10, wherein the immunogenic composition comprises Ct-089, Ct-858 and Ct-875.

12. The composition according to claim 2 wherein the immunogenic composition further comprises a pharmaceutically acceptable diluent or carrier.

13. The composition according to claim 2 wherein the immunogenic composition further comprises an adjuvant.

14. (canceled)

15. The composition according to claim 14 wherein the adjuvant comprises 3D-MPL, QS21 or a combination of 3D-MPL and QS21.

16.-17. (canceled)

18. An immunogenic composition comprising a fusion protein, where said fusion protein comprises two proteins selected from the group consisting of Swib, Momp, Ct-858, Ct-875, Ct-622, Ct-089, passenger domain of (PmpGpd) and passenger domain of PmpD (PmpDpd).

19. The composition according to claim 2, wherein the immunogenic composition comprises Ct-858, Ct-875, and a Chlamydia polypeptide selected from the group consisting of CT-089, CT-622, and PmpDpD.

20. The composition according to claim 2, wherein the immunogenic composition comprises Ct-858, Ct-875, and two Chlamydia polypeptides selected from the group consisting of Momp, PmpDpd, PmpGpd, Ct-622, and Swib.

21. An immunogenic composition according to claim 2 comprising Momp, Ct-089, Ct-858, Swib and PmpDpd polypeptides.

22. (canceled)

23. A method for the prevention of ocular Chlamydial infection by a second Chlamydia trachomatis serovar, comprising the administration of an immunogenic composition comprising a Chlamydial protein derived from a first Chlamydia trachomatis serovar, said protein selected from the group consisting of Ct-089, Ct-858 and Ct-875.

24.-28. (canceled)

29. The method according to claim 23, wherein the immunogenic composition comprises Ct-089, Ct-858 and Ct-875.

30. The method according to claim 23, wherein the first Chlamydia trachomatis serovar is selected from serovars A, B, Ba, C, D, Da, E, F, G, H, I, Ia, J, Ja, K, L1, L2 and L3.

31. The method according to claim 23, wherein the first Chlamydia trachomatis serovar is selected from the Chlamydia trachomatis ocular serovars.

32. The method according to claim 31, wherein the first Chlamydia trachomatis serovar is selected from the Chlamydia trachomatis ocular serovars A, B, Ba and C.

33. The method according to claim 23, wherein the first Chlamydia trachomatis serovar is selected from the Chlamydia trachomatis oculogenital serovars.

34. The method according to claim 33, wherein the first Chlamydia trachomatis serovar is selected from the Chlamydia trachomatis oculogenital serovars D, Da, E, F, G, H, I, Ia, J, Ja and K.

35. The method according to claim 23, wherein the first Chlamydia trachomatis serovar is selected from the Chlamydia trachomatis LGV serovars.

36. The method according to claim 35, wherein the first Chlamydia trachomatis serovar is selected from the Chlamydia trachomatis LGV serovars L1, L2 and L3.

37.-43. (canceled)

44. The method according to claim 23, wherein the immunogenic composition further comprises an adjuvant.

45. (canceled)

46. The method according to claim 45, wherein the adjuvant comprises 3D-MPL, QS21 or a combination of 3D-MPL and QS21.

47.-48. (canceled)


SEQUENCE LISTING (126 sequences)

SEQ ID NO: 1 - DNA, 261 bp, Chlamydia trachomatis
  misc_feature: Ct-460, also known as Swib, from Serovar LGVII (LII or L2)
atgagtcaaaataagaactctgctttcatgcagcctgtgaacgtatccgctgatttagctgccatcgttggtgcaggacctatgcctcgcacagagatcattaagaaaatgtgggattacattaaggagaatagtcttcaagatcctacaaacaaacgtaatatcaatcccgatgataaattggctaaagtttttggaactgaaaaacctatcgatatgttccaaatgacaaaaatggtttctcaacacatcattaaataa

SEQ ID NO: 2 - protein, 86 aa, Chlamydia trachomatis
  misc_feature: Ct-460, also known as Swib, from Serovar LGVII (LII or L2)
MSQNKNSAFMQPVNVSADLAAIVGAGPMPRTEIIKKMWDYIKENSLQDPTNKRNINPDDKLAKVFGTEKPIDMFQMTKMVSQHIIK

SEQ ID NO: 3 - DNA, 1122 bp, Chlamydia trachomatis
  misc_feature: antigen known as Major Outer Membrane Protein (MOMP) from serovar F
ctgcctgtggggaatcctgctgaaccaagccttatgatcgacggaattctgtgggaaggtttcggcggagatccttgcgatccttgcaccacttggtgtgacgctatcagcatgcgtatgggttactatggtgactttgttttcgaccgtgttttgaaaacagatgtgaataaagagtttgaaatgggcgaggctttagccggagcttctgggaatacgacctctactctttcaaaattggtagaacgaacgaaccctgcatatggcaagcatatgcaagacgcagagatgtttaccaatgccgcttgcatgacattgaatatttgggatcgttttgatgtattctgtacattaggagccaccagtggatatcttaaaggaaattcagcatctttcaacttagttgggttattcggcgatggtgtaaacgccacgaaacctgctgcagatagtattcctaacgtgcagttaaatcagtctgtggtggaactgtatacagatactacttttgcttggagtgttggagctcgtgcagctttgtgggaatgtggatgtgcaactttaggagcttctttccaatatgctcaatctaaacctaaaatcgaagaattaaacgttctctgtaacgcagcagagtttactattaataaacctaaagggtatgtaggtaaggagtttcctcttgatcttacagcaggaacagatgcagcgacgggcactaaagatgcctctattgattaccatgagtggcaagcaagtttatctctttcttacagactcaatatgttcactccctacattggagttaaatggtctcgtgcaagctttgattctgatacaattcgtatagcccagccgaggttggtaacacctgttgtagatattacaacccttaacccaactattgcaggatgcggcagtgtagctggagctaacacggaaggacagatatctgatacaatgcaaatcgtctccttgcaattgaacaagatgaaatctagaaaatcttgcggtattgcagtaggaacaactattgtggatgcagacaaatacgcagttacagttgagactcgcttgatcgatgagagagctgctcacgtaaatgcacaattccgcttctag

SEQ ID NO: 4 - protein, 373 aa, Chlamydia trachomatis
  misc_feature: antigen known as Major Outer Membrane Protein (MOMP) from serovar F
LPVGNPAEPSLMIDGILWEGFGGDPCDPCTTWCDAISMRMGYYGDFVFDRVLKTDVNKEFEMGEALAGASGNTTSTLSKLVERTNPAYGKHMQDAEMFTNAACMTLNIWDRFDVFCTLGATSGYLKGNSASFNLVGLFGDGVNATKPAADSIPNVQLNQSVVELYTDTTFAWSVGARAALWECGCATLGASFQYAQSKPKIEELNVLCNAAEFTINKPKGYVGKEFPLDLTAGTDAATGTKDASIDYHEWQASLSLSYRLNMFTPYIGVKWSRASFDSDTIRIAQPRLVTPVVDITTLNPTIAGCGSVAGANTEGQISDTMQIVSLQLNKMKSRKSCGIAVGTTIVDADKYAVTVETRLIDERAAHVNAQFRF

SEQ ID NO: 5 - DNA, 1746 bp, Chlamydia trachomatis
  misc_feature: Ct-858 from serovar E
gtacgaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtgctcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacacctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttaa

SEQ ID NO: 6 - protein, 581 aa, Chlamydia trachomatis
  misc_feature: Ct-858 from serovar E
VRGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 7 - DNA, 1776 bp, Chlamydia trachomatis
  misc_feature: Ct-875 from serovar E
atgagcatcaggggagtaggaggcaacgggaatagtcgaatcccttctcataatggggatggatcgaatcgcagaagtcaaaatacgaagggtaataataaagttgaagatcgagtttgttctctatattcatctcgtagtaacgaaaatagagaatctccttatgcagtagtagacgtcagctctatgatcgagagcaccccaacgagtggagagacgacaagagcttcgcgtggagtgctcagtcgtttccaaagaggtttagtacgaatagctgacaaagtaagacgagctgttcagtgtgcgtggagttcagtctctacaagcagatcgtctgcaacaagagccgcagaatccggatcaagtagtcgtactgctcgtggtgcaagttctgggtatagggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaaggagttggctggccctgacggggtagcggccgcccgggaaattgctaaaagatgggagaaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagtacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagagacctgatgaagcgttggaatagagaagtcgatagggaataa

SEQ ID NO: 8 - protein, 591 aa, Chlamydia trachomatis
  misc_feature: Ct-875 from serovar E
MSIRGVGGNGNSRIPSHNGDGSNRRSQNTKGNNKVEDRVCSLYSSRSNENRESPYAVVDVSSMIESTPTSGETTRASRGVLSRFQRGLVRIADKVRRAVQCAWSSVSTSRSSATRAAESGSSSRTARGASSGYREYSPSAARGLRLMFTDFWRTRVLRQTSPMAGVFGNLDVNEARLMAAYTSECADHLEAKELAGPDGVAAAREIAKRWEKRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRVQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMNMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMKRWNREVDRE

SEQ ID NO: 9 - DNA, 1962 bp, Chlamydia trachomatis
  misc_feature: Ct-622 from serovar E
atggaatcaggaccagaatcagtttcttctaatcagagctcgatgaatccaattattaatgggcaaatcgcttctaattcggagaccaaagagtccacgaaggcgtccgaagcgagtccttcagcatcgtcctctgtaagcagctggagttttttatcctcagcaaagaatgcattaatctctcttcgtgatgccatcttgaataaaaattccagtccaacagactctctctctcaattagaggcctctacttctacctctacggttacacgtgtagcggcaaaagattatgatgaggctaaatcgaattttgatacggcgaaaagtggattagagaacgctaagacacttgctgaatacgaaacgaaaatggctgatttgatggcagctctccaagatatggagcgtttagctaattcagatcctagtaacaatcataccgaagaagtaaataatattaagaaagcgctcgaagcacaaaaagatactattgataagctgaataaactcgttacgctgcaaaatcagaataaatctttaacagaagtgttgaaaacaactgactctgcagatcagattccagcgattaatagtcagttagagatcaacaaaaattctgcagatcaaattatcaaagatctggaaagacaaaacataagttatgaagctgttctcactaacgcaggagaggttatcaaagcttcttctgaagcgggaattaagttaggacaagctttgcagtctattgtggatgctggggaccaaagtcaggctgcagttctgcaagcacagcaaaataatagcccagataatattgcagccacgaaggaattaattgatgctgctgaaacgaaggtaaacgagttaaaacaagagcatacagggctaacggactcgcctttagtgaaaaaagctgaggagcagattagtcaagcacaaaaagatattcaagagatcaaacctagtggttcggatattcctatcgttggtccgagtgggtcagctgcttccgcaggaagtgcggcaggagcgttgaaatcctctaacaattcaggaagaatttccttgttgcttgatgatgtagacaatgaaatggcagcgattgcactgcaaggttttcgatctatgatcgaacaatttaatgtaaacaatcctgcaacagctaaagagctacaagctatggaggctcagctgactgcgatgtcagatcaactggttggtgcggatggcgagctcccagccgaaatacaagcaatcaaagatgctcttgcgcaagctttgaaacaaccatcagcagatggtttggctacagctatgggacaagtggcttttgcagctgccaaggttggaggaggctccgcaggaacagctggcactgtccagatgaatgtaaaacagctttacaagacagcgttttcttcgacttcttccagctcttatgcagcagcactttccgatggatattctgcttacaaaacactgaactctttatattccgaaagcagaagcggcgtgcagtcagctattagtcaaactgcaaatcccgcgctttccagaagcgtttctcgttctggcatagaaagtcaaggacgcagtgcagatgctagccaaagagcagcagaaactattgtcagagatagccaaacgttaggtgatgtatatagccgcttacaggttctggattctttgatgtctacgattgtgagcaatccgcaagcaaatcaagaagagattatgcagaagctcacggcatctattagcaaagctccacaatttgggtatcctgctgttcagaattctgcggatagcttgcagaagtttgctgcgcaattggaaagagagtttgttgatggggaacgtagtctcgcagaatctcaagagaatgcgtttagaaaacagcccgctttcattcaacaggtgttggtaaacattgcttctctattctctggttatctttcttaa

SEQ ID NO: 10 - protein, 653 aa, Chlamydia trachomatis
  misc_feature: Ct-622 from serovar E
MESGPESVSSNQSSMNPIINGQIASNSETKESTKASEASPSASSSVSSWSFLSSAKNALISLRDAILNKNSSPTDSLSQLEASTSTSTVTRVAAKDYDEAKSNFDTAKSGLENAKTLAEYETKMADLMAALQDMERLANSDPSNNHTEEVNNIKKALEAQKDTIDKLNKLVTLQNQNKSLTEVLKTTDSADQIPAINSQLEINKNSADQIIKDLERQNISYEAVLTNAGEVIKASSEAGIKLGQALQSIVDAGDQSQAAVLQAQQNNSPDNIAATKELIDAAETKVNELKQEHTGLTDSPLVKKAEEQISQAQKDIQEIKPSGSDIPIVGPSGSAASAGSAAGALKSSNNSGRISLLLDDVDNEMAAIALQGFRSMIEQFNVNNPATAKELQAMEAQLTAMSDQLVGADGELPAEIQAIKDALAQALKQPSADGLATAMGQVAFAAAKVGGGSAGTAGTVQMNVKQLYKTAFSSTSSSSYAAALSDGYSAYKTLNSLYSESRSGVQSAISQTANPALSRSVSRSGIESQGRSADASQRAAETIVRDSQTLGDVYSRLQVLDSLMSTIVSNPQANQEEIMQKLTASISKAPQFGYPAVQNSADSLQKFAAQLEREFVDGERSLAESQENAFRKQPAFIQQVLVNIASLFSGYLS

SEQ ID NO: 11 - DNA, 2010 bp, Chlamydia trachomatis
  misc_feature: passenger domain of PmpG (Ct-871) from serovar LGVII
gcagaaatcatgattcctcaaggaatttacgatggggagacgttaactgtatcatttccctatactgttataggagatccgagtgggactactgttttttctgcaggagagttaacattaaaaaatcttgacaattctattgcagctttgcctttaagttgttttgggaacttattagggagttttactgttttagggagaggacactcgttgactttcgagaacatacggacttctacaaatggggcagctctaagtaatagcgctgctgatggactgtttactattgagggttttaaagaattatccttttccaattgcaattcattacttgccgtactgcctgctgcaacgactaataagggtagccagactccgacgacaacatctacaccgtctaatggtactatttattctaaaacagatcttttgttactcaataatgagaagttctcattctatagtaatttagtctctggagatgggggagctatagatgctaagagcttaacggttcaaggaattagcaagctttgtgtcttccaagaaaatactgctcaagctgatgggggagcttgtcaagtagtcaccagtttctctgctatggctaacgaggctcctattgcctttgtagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctaagcaaccaacaagtggacaggcttctaatacgagtaataattacggagatggaggagctatcttctgtaagaatggtgcgcaagcaggatccaataactctggatcagtttcctttgatggagagggagtagttttctttagtagcaatgtagctgctgggaaagggggagctatttatgccaaaaagctctcggttgctaactgtggccctgtacaatttttaaggaatatcgctaatgatggtggagcgatttatttaggagaatctggagagctcagtttatctgctgattatggagatattattttcgatgggaatcttaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccaaacttctaaaaattaacgatggtgaaggatacacaggggatattgtttttgctaatggaagcagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcagtcattggtagcacaactgctggttctgttacaattagtgggcctatcttttttgaggatttggatgatacagcttatgataggtatgattggctaggttctaatcaaaaaatcaatgtcctgaaattacagttagggactaagcccccagctaatgccccatcagatttgactctagggaatgagatgcctaagtatggctatcaaggaagctggaagcttgcgtgggatcctaatacagcaaataatggtccttatactctgaaagctacatggactaaaactgggtaa

SEQ ID NO: 12 - protein, 669 aa, Chlamydia trachomatis
  misc_feature: passenger domain of PmpG (Ct-871) from serovar LGVII
AEIMIPQGIYDGETLTVSFPYTVIGDPSGTTVFSAGELTLKNLDNSIAALPLSCFGNLLGSFTVLGRGHSLTFENIRTSTNGAALSNSAADGLFTIEGFKELSFSNCNSLLAVLPAATTNKGSQTPTTTSTPSNGTIYSKTDLLLLNNEKFSFYSNLVSGDGGAIDAKSLTVQGISKLCVFQENTAQADGGACQVVTSFSAMANEAPIAFVANVAGVRGGGIAAVQDGQQGVSSSTSTEDPVVSFSRNTAVEFDGNVARVGGGIYSYGNVAFLNNGKTLFLNNVASPVYIAAKQPTSGQASNTSNNYGDGGAIFCKNGAQAGSNNSGSVSFDGEGVVFFSSNVAAGKGGAIYAKKLSVANCGPVQFLRNIANDGGAIYLGESGELSLSADYGDIIFDGNLKRTAKENAADVNGVTVSSQAISMGSGGKITTLRAKAGHQILFNDPIEMANGNNQPAQSSKLLKINDGEGYTGDIVFANGSSTLYQNVTIEQGRIVLREKAKLSVNSLSQTGGSLYMEAGSTLDFVTPQPPQQPPAANQLITLSNLHLSLSSLLANNAVTNPPTNPPAQDSHPAVIGSTTAGSVTISGPIFFEDLDDTAYDRYDWLGSNQKINVLKLQLGTKPPANAPSDLTLGNEMPKYGYQGSWKLAWDPNTANNGPYTLKATWTKTG

SEQ ID NO: 13 - DNA, 3519 bp, Chlamydia trachomatis
  misc_feature: passenger domain of PmpD (Ct-812) from serovar LGVII
agttgcgtagatcttcatgctggaggacagtctgtaaatgagctggtatatgtaggccctcaagcggttttattgttagaccaaattcgagatctattcgttgggtctaaagatagtcaggctgaaggacagtataggttaattgtaggagatccaagttctttccaagagaaagatgcagatactcttcccgggaaggtagagcaaagtactttgttctcagtaaccaatcccgtggttttccaaggtgtggaccaacaggatcaagtctcttcccaagggttaatttgtagttttacgagcagcaaccttgattctccccgtgacggagaatcttttttaggtattgcttttgttggggatagtagtaaggctggaatcacattaactgacgtgaaagcttctttgtctggagcggctttatattctacagaagatcttatctttgaaaagattaagggtggattggaatttgcatcatgttcttctctagaacaggggggagcttgtgcagctcaaagtattttgattcatgattgtcaaggattgcaggttaaacactgtactacagccgtgaatgctgaggggtctagtgcgaatgatcatcttggatttggaggaggcgctttctttgttacgggttctctttctggagagaaaagtctctatatgcctgcaggagatatggtagttgcgaattgtgatggggctatatcttttgaaggaaacagcgcgaactttgctaatggaggagcgattgctgcctctgggaaagtgctttttgtcgctaatgataaaaagacttcttttatagagaaccgagctttgtctggaggagcgattgcagcctcttctgatattgcctttcaaaactgcgcagaactagttttcaaaggcaattgtgcaattggaacagaggataaaggttctttaggtggaggggctatatcttctctaggcaccgttcttttgcaagggaatcacgggataacttgtgataagaatgagtctgcttcgcaaggaggcgccatttttggcaaaaattgtcagatttctgacaacgaggggccagtggttttcagagatagtacagcttgcttaggaggaggcgctattgcagctcaagaaattgtttctattcagaacaatcaggctgggatttccttcgagggaggtaaggctagtttcggaggaggtattgcgtgtggatctttttcttccgcaggcggtgcttctgttttagggactattgatatttcgaagaatttaggcgcgatttcgttctctcgtactttatgtacgacctcagatttaggacaaatggagtaccagggaggaggagctctatttggtgaaaatatttctctttctgagaatgctggtgtgctcacctttaaagacaacattgtgaagacttttgcttcgaatgggaaaattctgggaggaggagcgattttagctactggtaaggtggaaattaccaataattccggaggaatttcttttacaggaaatgcgagagctccacaagctcttccaactcaagaggagtttcctttattcagcaaaaaagaagggcgaccactctcttcaggatattctgggggaggagcgattttaggaagagaagtagctattctccacaacgctgcagtagtatttgagcaaaatcgtttgcagtgcagcgaagaagaagcgacattattaggttgttgtggaggaggcgctgttcatgggatggatagcacttcgattgttggcaactcttcagtaagatttggtaataattacgcaatgggacaaggagtctcaggaggagctcttttatctaaaacagtgcagttagctggaaatggaagcgtcgatttttctcgaaatattgctagtttgggaggaggagctcttcaagcttctgaaggaaattgtgagctagttgataacggctatgtgctattcagagataatcgagggagggtttatgggggtgctatttcttgcttacgtggagatgtagtcatttctggaaacaagggtagagttgaatttaaagacaacatagcaacacgtctttatgtggaagaaactgtagaaaaggttgaagaggtagagccagctcctgagcaaaaagacaataatgagctttctttcttagggagtgtagaacagagttttattactgcagctaatcaagctcttttcgcatctgaagatggggatttatcacctgagtcatccatttcttctgaagaacttgcgaaaagaagagagtgtgctggaggagctatttttgcaaaacgggttcgtattgtagataaccaagaggccgttgtattctcgaataacttctctgatatttatggcggcgccatttttacaggttctcttcgagaagaggataagttagatgggcaaatccctgaagtcttgatctcaggcaatgcaggggatgttgttttttccggaaattcctcgaagcgtgatgagcatcttcctcatacaggtgggggagccatttgtactcaaaatttgacgatttctcagaatacagggaatgttctgttttataacaacgtggcctgttcgggaggagctgttcgtatagaggatcatggtaatgttcttttagaagcttttggaggagatattgtttttaaaggaaattcttctttcagagcacaaggatccgatgctatctattttgcaggtaaagaatcgcatattacagccctgaatgctacggaaggacatgctattgttttccacgacgcattagtttttgaaaatctaaaagaaaggaaatctgctgaagtattgttaatcaatagtcgagaaaatccaggttacactggatctattcgatttttagaagcagaaagtaaagttcctcaatgtattcatgtacaacaaggaagccttgagttgctaaatggagctacattatgtagttatggttttaaacaagatgctggagctaagttggtattggctgctggatctaaactgaagattttagattcaggaactcctgtacaagggcatgctatcagtaaacctgaagcagaaatcgagtcatcttctgaaccagagggtgcacattctctttggattgcgaagaatgctcaaacaacagttcctatggttgatatccatactatttctgtagatttagcctccttctcttctagtcaacaggaggggacagtagaagctcctcaggttattgttcctggaggaagttatgttcgatctggagagcttaatttggagttagttaacacaacaggtactggttatgaaaatcatgctttgttgaagaatgaggctaaagttccattgatgtctttcgttgcttctagtgatgaagcttcagccgaaatcagtaacttgtcggtttctgatttacagattcatgtagcaactccagagattgaagaagacacatacggccatatgggagattggtctgaggctaaaattcaagatggaactcttgtcattagttggaatcctactggataa

SEQ ID NO: 14 - protein, 1172 aa, Chlamydia trachomatis
  misc_feature: passenger domain of PmpD (Ct-812) from serovar LGVII
SCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSGGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGSVEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLKERKSAEVLLINSRENPGYTGSIRFLEAESKVPQCIHVQQGSLELLNGATLCSYGFKQDAGAKLVLAAGSKLKILDSGTPVQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASSDEASAEISNLSVSDLQIHVATPEIEEDTYGHMGDWSEAKIQDGTLVISWNPTG

SEQ ID NO: 15 - DNA, 1266 bp, Chlamydia trachomatis
  misc_feature: Ct-089 from serovar E
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgaagaaattctcgatgcgctcacaagtaaattttctgatcccacaataaaggatctagctcttgattatctaattcaaacagctccctctgataggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatctccaagtaacctcatccccctctaattgtgataatttacgtcaaatgcttgcttcttacttgccatcagagaaaaccgctgttatggagtttctagtaaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 16 - protein, 421 aa, Chlamydia trachomatis
  misc_feature: Ct-089 from serovar E
MTASGGAGGLGSTQTVDVARAQAAAATQDAQEVIGSQEASEASMLKGCEDLINPAAATRIKKKEEKFESLEARRKPTADKAEKKSESTEEKGDTPLEDRFTEDLSEVSGEDFRGLKNSFDDDSSPEEILDALTSKFSDPTIKDLALDYLIQTAPSDRKLKSALIQAKHQLMSQNPQAIVGGRNVLLASETFASRANTSPSSLRSLYLQVTSSPSNCDNLRQMLASYLPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 17-20 - skipped (4 entries)

SEQ ID NO: 21 - DNA, 1776 bp, Chlamydia trachomatis
  misc_feature: Ct-875 from serovar D
atgagcatcaggggagtaggaggcaacgggaatagtcgaatcccttctcataatggggatggatcgaatcgcagaagtcaaaatacgaagggtaataataaagttgaagatcgagtttgttctctatattcatctcgtagtaacgaaaatagagaatctccttatgcagtagtagacgtcagctctatgatcgagagcaccccaacgagtggagagacgacaagagcttcgcgtggagtgttcagtcgtttccaaagaggtttagtacgagtagctgacaaagtaagacgagctgttcagtgtgcgtggagttcagtctctacaagaagatcgtctgcaacaagagccgcagaatccggatcaagtagtcgtactgctcgtggtgcaagttctgggtatagggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaacaagttggctggccctgacggggtagcggccgcccgggaaattgctaaaagatgggagcaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagcacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatggatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagagacctgatgaggcgttggaatagagaagtcgatagggaataa

SEQ ID NO: 22 - protein, 591 aa, Chlamydia trachomatis
  misc_feature: Ct-875 from serovar D
MSIRGVGGNGNSRIPSHNGDGSNRRSQNTKGNNKVEDRVCSLYSSRSNENRESPYAVVDVSSMIESTPTSGETTRASRGVFSRFQRGLVRVADKVRRAVQCAWSSVSTRRSSATRAAESGSSSRTARGASSGYREYSPSAARGLRLMFTDFWRTRVLRQTSPMAGVFGNLDVNEARLMAAYTSECADHLEANKLAGPDGVAAAREIAKRWEQRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMDMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMRRWNREVDRE

SEQ ID NO: 23-26 - skipped (4 entries)

SEQ ID NO: 27 - DNA, 3042 bp, Chlamydia trachomatis
  misc_feature: PmpG (Ct-871) from serovar D
atgcaaacgtctttccataagttctttctttcaatgattctagcttattcttgctgctctttaagtgggggggggtatgcagcagaaatcatgattcctcaaggaatttacgatggggagacgttaactgtatcatttccctatactgttataggagatccgagtgggactactgttttttctgcaggagagttaacgttaaaaaatcttgacaattctattgcagctttgcctttaagttgttttgggaacttattagggagttttactgttttagggagaggacactcgttgactttcgagaacatacggacttctacaaatggagctgcactaagtgacagcgctaatagcgggttatttactattgagggttttaaagaattatctttttccaattgcaactcattacttgccgtactgcctgctgcaacgactaataatggtagccagactccgacgacaacatctacaccgtctaatggtactatttattctaaaacagatcttttgttactcaataatgagaagttctcattctatagtaatttagtctctggagatgggggagctatagatgctaagagcttaacggttcaaggaattagcaagctttgtgtcttccaagaaaatactgctcaagctgatgggggagcttgtcaagtagtcaccagtttctctgctatggctaacgaggctcctattgcctttatagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctgagcaaccaacaaatggacaggcttctaatacgagtgataattacggagatggaggagctatcttctgtaagaatggtgcgcaagcagcaggatccaataactctggatcagtttcctttgatggagagggagtagttttctttagtagcaatgtagctgctgggaaagggggagctatttatgccaaaaagctctcggttgctaactgtggccctgtacaattcttagggaatatcgctaatgatggtggagcgatttatttaggagaatctggagagctcagtttatctgctgattatggagatattattttcgatgggaatcttaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccgaacctctaaaaattaacgatggtgaaggatacacaggggatattgtttttgctaatggaaacagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcaatcattggtagcacaactgctggttctgttacaattagtgggcctatcttttttgaggatttggatgatacagcttatgataggtatgattggctaggttctaatcaaaaaatcgatgtcctgaaattacagttagggactcagccctcagctaatgccccatcagatttgactctagggaatgagatgcctaagtatggctatcaaggaagctggaagcttgcgtgggatcctaatacagcaaataatggtccttatactctgaaagctacatggactaaaactgggtataatcctgggcctgagcgagtagcttctttggttccaaatagtttatggggatccattttagatatacgatctgcgcattcagcaattcaagcaagtgtggatgggcgctcttattgtcgaggattatgggtttctggagtttcgaatttcttctatcatgaccgcgatgctttaggtcagggatatcggtatattagtgggggttattccttaggagcaaactcctactttggatcatcgatgtttggtctagcatttaccgaagtatttggtagatctaaagattatgtagtgtgtcgttccaatcatcatgcttgcataggatccgtttatctatctaccaaacaagctttatgtggatcctatttgttcggagatgcgtttatccgtgctagctacgggtttgggaaccagcatatgaaaacctcatacacatttgcagaggagagcgatgttcgttgggataataactgtctggttggagagattggagtgggattaccgattgtgattactccatctaagctctatttgaatgagttgcgtcctttcgtgcaagctgagttttcttatgccgatcatgaatcttttacagaggaaggcgatcaagctcgggcattcaggagtggacatctcatgaatctatcagttcctgttggagtaaaatttgatcgatgttctagtacacaccctaataaatatagctttatgggggcttatatctgtgatgcttatcgcaccatctctgggactcagacaacactcctatcccatcaagagacatggacaacagatgcctttcatttggcaagacatggagtcatagttagagggtctatgtatgcttctctaacaagcaatatagaagtatatggccatggaagatatgagtatcgagatacttctcgaggttatggtttgagtgcaggaagtaaagtccggttctaa

SEQ ID NO: 28 - protein, 1013 aa, Chlamydia trachomatis
  misc_feature: PmpG (Ct-871) from serovar D
MQTSFHKFFLSMILAYSCCSLSGGGYAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLYQNVTIEQGRIVLREKAKLSVNSLSQTGGSLYMEAGSTLDFVTPQPPQQPPAANQLITLSNLHLSLSSLLANNAVTNPPTNPPAQDSHPAIIGSTTAGSVTISGPIFFEDLDDTAYDRYDWLGSNQKIDVLKLQLGTQPSANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFTEVFGRSKDYVVCRSNHHACIGSVYLSTKQALCGSYLFGDAFIRASYGFGNQHMKTSYTFAEESDVRWDNNCLVGEIGVGLPIVITPSKLYLNELRPFVQAEFSYADHESFTEEGDQARAFRSGHLMNLSVPVGVKFDRCSSTHPNKYSFMGAYICDAYRTISGTQTTLLSHQETWTTDAFHLARHGVIVRGSMYASLTSNIEVYGHGRYEYRDTSRGYGLSAGSKVRF

SEQ ID NO: 29-32 - skipped (4 entries)

SEQ ID NO: 33 - DNA, 1806 bp, Chlamydia trachomatis
  misc_feature: Ct-858 from serovar D
atgaaaatgaataggatttggctattactgcttaccttttcttctgccatacattctcctgtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttaa

SEQ ID NO: 34 - protein, 601 aa, Chlamydia trachomatis
  misc_feature: Ct-858 from serovar D
MKMNRIWLLLLTFSSAIHSPVQGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 35-40 - skipped (6 entries)

SEQ ID NO: 41 - DNA, 4596 bp, Chlamydia trachomatis
  misc_feature: PmpD (Ct-812) from serovar D
atgagttccgagaaagatataaaaagcacctgttctaagttttctttgtctgtagtagcagctatccttgcctctgttagcgggttagctagttgcgtagatcttcatgctggaggacagtctgtaaatgagctggtatatgtaggccctcaagcggttttattgttagaccaaattcgagatctattcgttgggtctaaagatagtcaggctgaaggacagtataggttaattgtaggagatccaagttctttccaagagaaagatgcggatactcttcccgggaaggtagagcaaagtactttgttctcagtaaccaatcccgtggttttccaaggtgtggaccaacaggatcaagtctcttcccaagggttaatttgtagttttacgagcagcaaccttgattctcctcgtgacggagaatcttttttaggtattgcttttgttggggatagtagtaaggctggaatcacattaactgacgtgaaagcttctttgtctggagcggctttatattctacagaagatcttatctttgaaaagattaagggtggattggaatttgcatcatgttcttctctagaacaggggggagcttgtgcagctcaaagtattttgattcatgattgtcaaggattgcaggttaaacactgtactacagccgtgaatgctgaggggtctagtgcgaatgatcatcttggatttggaggaggcgctttctttgttacgggttctctttctggagagaaaagtctctatatgcctgcaggagatatggtagttgcgaattgtgatggggctatatcttttgaaggaaacagcgcgaactttgctaatggaggagcgattgctgcctctgggaaagtgctttttgtcgctaatgataaaaagacttcttttatagagaaccgagctttgtctggaggagcgattgcagcctcttctgatattgcctttcaaaactgcgcagaactagttttcaaaggcaattgtgcaattggaacagaggataaaggttctttaggtggaggggctatatcttctctaggcaccgttcttttgcaagggaatcacgggataacttgtgataagaatgagtctgcttcgcaaggaggcgccatttttggcaaaaattgtcagatttctgacaacgaggggccagtggttttcagagatagtacagcttgcttaggaggaggcgctattgcagctcaagaaattgtttctattcagaacaatcaggctgggatttccttcgagggaggtaaggctagtttcggaggaggtattgcgtgtggatctttttcttccgcaggtggtgcttctgttttagggaccattgatatttcgaagaatttaggcgcgatttcgttctctcgtactttatgtacgacctcagatttaggacaaatggagtaccagggaggaggagctctatttggtgaaaatatttctctttctgagaatgctggtgtgctcacctttaaagacaacattgtgaagacttttgcttcgaatgggaaaattctgggaggaggagcgattttagctactggtaaggtggaaattactaataattccgaaggaatttcttttacaggaaatgcgagagctccacaagctcttccaactcaagaggagtttcctttattcagcaaaaaagaagggcgaccactctcttcaggatattctgggggaggagcgattttaggaagagaagtagctattctccacaacgctgcagtagtatttgagcaaaatcgtttgcagtgcagcgaagaagaagcgacattattaggttgttgtggaggaggcgctgttcatgggatggatagcacttcgattgttggcaactcttcagtaagatttggtaataattacgcaatgggacaaggagtctcaggaggagctcttttatctaaaacagtgcagttagctgggaatggaagcgtcgatttttctcgaaatattgctagtttgggaggaggagctcttcaagcttctgaaggaaattgtgagctagttgataacggctatgtgctattcagagataatcgagggagggtttatgggggtgctatttcttgcttacgtggagatgtagtcatttctggaaacaagggtagagttgaatttaaagacaacatagcaacacgtctttatgtggaagaaactgtagaaaaggttgaagaggtagagccagctcctgagcaaaaagacaataatgagctttctttcttagggagagcagaacagagttttattactgcagctaatcaagctcttttcgcatctgaagatggggatttatcacctgagtcatccatttcttctgaagaacttgcgaaaagaagagagtgtgctggaggagctatttttgcaaaacgggttcgtattgtagataaccaagaggccgttgtattctcgaataacttctctgatatttatggcggcgccatttttacaggttctcttcgagaagaggataagttagatgggcaaatccctgaagtcttgatctcaggcaatgcaggggatgttgttttttccggaaattcctcgaagcgtgatgagcatcttcctcatacaggtgggggagccatttgtactcaaaatttgacgatttctcagaatacagggaatgttctgttttataacaacgtggcctgttcgggaggagctgttcgtatagaggatcatggtaatgttcttttagaagcttttggaggagatattgtttttaaaggaaattcttctttcagagcacaaggatccgatgctatctattttgcaggtaaagaatcgcatattacagccctgaatgctacggaaggacatgctattgttttccacgacgcattagtttttgaaaatctagaagaaaggaaatctgctgaagtattgttaatcaatagtcgagaaaatccaggttacactggatctattcgatttttagaagcagaaagtaaagttcctcaatgtattcatgtacaacaaggaagccttgagttgctaaatggagccacattatgtagttatggttttaaacaagatgctggagctaagttggtattggctgctggagctaaactgaagattttagattcaggaactcctgtacaacaagggcatgctatcagtaaacctgaagcagaaatcgagtcatcttctgaaccagagggtgcacattctctttggattgcgaagaatgctcaaacaacagttcctatggttgatatccatactatttctgtagatttagcctccttctcttctagtcaacaggaggggacagtagaagctcctcaggttattgttcctggaggaagttatgttcgatctggagagcttaatttggagttagttaacacaacaggtactggttatgaaaatcatgctttattgaagaatgaggctaaagttccattgatgtctttcgttgcttctggtgatgaagcttcagccgaaatcagtaacttgtcggtttctgatttacagattcatgtagtaactccagagattgaagaagacacatacggccatatgggagattggtctgaggctaaaattcaagatggaactcttgtcattagttggaatcctactggatatcgattagatcctcaaaaagcaggggctttagtatttaatgcattatgggaagaaggggctgtcttgtctgctctgaaaaatgcacgctttgctcataatctcactgctcagcgtatggaattcgattattctacaaatgtgtggggattcgcctttggtggtttccgaactctatctgcagagaatctggttgctattgatggatacaaaggagcttatggtggtgcttctgctggagtcgatattcaattgatggaagattttgttctaggagttagtggagctgctttcctaggtaaaatggatagtcagaagtttgatgcggaggtttctcggaagggagttgttggttctgtatatacaggatttttagctggatcctggttcttcaaaggacaatatagccttggagaaacacagaacgatatgaaaacgcgttatggagtactaggagagtcgagtgcttcttggacatctcgaggagtactggcagatgctttagttgaataccgaagtttagttggtcctgtgagacctactttttatgctttgcatttcaatccttatgtcgaagtatcttatgcttctatgaaattccctggctttacagaacaaggaagagaagcgcgttcttttgaagacgcttcccttaccaatatcaccattcctttagggatgaagtttgaattggcgttcataaaaggacagttttcagaggtgaactctttgggaataagttatgcatgggaagcttatcgaaaagtagaaggaggcgcggtgcagcttttagaagctgggtttgattgggagggagctccaatggatcttcctagacaggagctgcgtgtcgctctggaaaataatacggaatggagttcttacttcagcacagtcttaggattaacagctttttgtggaggatttacttctacagatagtaaactaggatatgaggcgaatactggattgcgattgatcttttaa

SEQ ID NO: 42 - protein, 1531 aa, Chlamydia trachomatis
  misc_feature: PmpD (Ct-812) from serovar D
MSSEKDIKSTCSKFSLSVVAAILASVSGLASCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRMEFDYSTNVWGFAFGGFRTLSAENLVAIDGYKGAYGGASAGVDIQLMEDFVLGVSGAAFLGKMDSQKFDAEVSRKGVVGSVYTGFLAGSWFFKGQYSLGETQNDMKTRYGVLGESSASWTSRGVLADALVEYRSLVGPVRPTFYALHFNPYVEVSYASMKFPGFTEQGREARSFEDASLTNITIPLGMKFELAFIKGQFSEVNSLGISYAWEAYRKVEGGAVQLLEAGFDWEGAPMDLPRQELRVALENNTEWSSYFSTVLGLTAFCGGFTSTDSKLGYEANTGLRLIF

SEQ ID NO: 43-46 - skipped (4 entries)

SEQ ID NO: 47 - DNA, 1185 bp, Chlamydia trachomatis
  misc_feature: Major Outer Membrane Protein (Momp) or Ct-681 from serovar LGVII
atgaaaaaactcttgaaatcggtattagtgtttgccgctttgagttctgcttcctccttgcaagctctgcctgtggggaatcctgctgaaccaagccttatgatcgacggaattctatgggaaggtttcggcggagatccttgcgatccttgcaccacttggtgtgacgctatcagcatgcgtatgggttactatggtgactttgttttcgaccgtgttttgcaaacagatgtgaataaagaattccaaatgggtgccaagcctacaactgctacaggcaatgctgcagctccatccacttgtacagcaagagagaatcctgcttacggccgacatatgcaggatgctgagatgtttacaaatgctgcttacatggcattgaatatttgggatcgttttgatgtattctgtacattaggagccaccagtggatatcttaaaggaaattcagcatctttcaacttagttggcttattcggagataatgagaaccatgctacagtttcagatagtaagcttgtaccaaatatgagcttagatcaatctgttgttgagttgtatacagatactacttttgcttggagtgctggagctcgtgcagctttgtgggaatgtggatgcgcgactttaggcgcttctttccaatacgctcaatccaagcctaaagtcgaagaattaaacgttctctgtaacgcagctgagtttactatcaataagcctaaaggatatgtagggcaagaattccctcttgatcttaaagcaggaacagatggtgtgacaggaactaaggatgcctctattgattaccatgaatggcaagcaagtttagctctctcttacagactgaatatgttcactccctacattggagttaaatggtctcgagcaagttttgatgcagacacgattcgtattgctcagccgaagtcagctacaactgtctttgatgttaccactctgaacccaactattgctggagctggcgatgtgaaagctagcgcagagggtcagctcggagataccatgcaaatcgtttccttgcaattgaacaagatgaaatctagaaaatcttgcggtattgcagtaggaacaactattgtggatgcagacaaatacgcagttacagttgagactcgcttgatcgatgagagagctgctcacgtaaatgcacaattccgcttctaa

SEQ ID NO: 48 - protein, 394 aa, Chlamydia trachomatis
  misc_feature: Major Outer Membrane Protein (Momp) or Ct-681 from serovar LGVII
MKKLLKSVLVFAALSSASSLQALPVGNPAEPSLMIDGILWEGFGGDPCDPCTTWCDAISMRMGYYGDFVFDRVLQTDVNKEFQMGAKPTTATGNAAAPSTCTARENPAYGRHMQDAEMFTNAAYMALNIWDRFDVFCTLGATSGYLKGNSASFNLVGLFGDNENHATVSDSKLVPNMSLDQSVVELYTDTTFAWSAGARAALWECGCATLGASFQYAQSKPKVEELNVLCNAAEFTINKPKGYVGQEFPLDLKAGTDGVTGTKDASIDYHEWQASLALSYRLNMFTPYIGVKWSRASFDADTIRIAQPKSATTVFDVTTLNPTIAGAGDVKASAEGQLGDTMQIVSLQLNKMKSRKSCGIAVGTTIVDADKYAVTVETRLIDERAAHVNAQFRF

SEQ ID NO: 49 - DNA, 1194 bp, Chlamydia trachomatis
  misc_feature: Major Outer Membrane Protein (Momp) or Ct-681 from serovar J
atgaaaaaactcttgaaatcggtattagtatttgccgctttgagttctgcttcctccttgcaagctctgcctgtggggaatcctgctgaaccaagccttatgatcgacggaattctgtgggaaggtttcggtggagatccttgcgatccttgcaccacttggtgtgacgctatcagcatgcgtatgggttactatggtgactttgttttcgaccgtgttttgaaaacagatgtgaataaagaatttcagatgggagcggcgcctactaccagcgatgtagcaggcttacaaaacgatccaacaacaaatgttgctcgtccaaatcccgcttatggcaaacacatgcaagatgctgaaatgtttacgaacgctgcttacatggcattaaatatctgggatcgttttgatgtattttgtacattgggagcaactaccggttatttaaaaggaaactccgcttccttcaacttagttggattattcggaacaaaaacacaagcttctagctttaatacagcgaatctttttcctaacactgctttgaatcaagctgtggttgagctttatacagacactacctttgcttggagcgtaggtgctcgtgcagctctctgggaatgtgggtgtgcaacgttaggagcttctttccaatatgctcaatctaaacctaaagtagaagagttaaatgttctttgtaatgcatccgaatttactattaataagccgaaaggatatgttggggcggaatttccacttgatattaccgcaggaacagaagctgcgacagggactaaggatgcctctattgactaccatgagtggcaagcaagtttagccctttcttacagattaaatatgttcactccttacattggagttaaatggtctagagtaagttttgatgccgacacgatccgtatcgctcagcctaaattggctgaagcaatcttggatgtcactactctaaacccgaccatcgctggtaaaggaactgtggtcgcttccggaagcgaaaacgacctggctgatacaatgcaaatcgtttccttgcagttgaacaagatgaaatctagaaaatcttgcggtattgcagtaggaacgactattgtagatgcagacaaatacgcagttacagttgagactcgcttgatcgatgagagagcagctcacgtaaatgcacaattccgcttctaa

SEQ ID NO: 50 - protein, 397 aa, Chlamydia trachomatis
  misc_feature: Major Outer Membrane Protein (Momp) or Ct-681 from serovar J
MKKLLKSVLVFAALSSASSLQALPVGNPAEPSLMIDGILWEGFGGDPCDPCTTWCDAISMRMGYYGDFVFDRVLKTDVNKEFQMGAAPTTSDVAGLQNDPTTNVARPNPAYGKHMQDAEMFTNAAYMALNIWDRFDVFCTLGATTGYLKGNSASFNLVGLFGTKTQASSFNTANLFPNTALNQAVVELYTDTTFAWSVGARAALWECGCATLGASFQYAQSKPKVEELNVLCNASEFTINKPKGYVGAEFPLDITAGTEAATGTKDASIDYHEWQASLALSYRLNMFTPYIGVKWSRVSFDADTIRIAQPKLAEAILDVTTLNPTIAGKGTVVASGSENDLADTMQIVSLQLNKMKSRKSCGIAVGTTIVDADKYAVTVETRLIDERAAHVNAQFRF

SEQ ID NO: 51 - DNA, 1194 bp, Chlamydia trachomatis
  misc_feature: Major Outer Membrane Protein (Momp) or Ct-681 from serovar H
atgaaaaaactcttgaaatcggtattagtatttgccgctttgagttctgcttcctccttgcaagctctgcctgtggggaatcctgctgaaccaagccttatgatcgacggaattctgtgggaaggttttggcggagatccttgcgatccttgcgccacttggtgtgacgctatcagcatgcgtgttggttactacggagactttgttttcgaccgtgttttgaaaactgatgtgaataaagaatttcagatgggagcggcgcctactaccaacgatgcagcagacttacaaaacgatccaaaaacaaatgttgctcgtccaaatcccgcttatggcaaacacatgcaagatgctgaaatgtttacgaacgctgcttacatggcattaaatatctgggatcgttttgatgtattttgtacattgggagcaactaccggttatttaaaaggaaactccgcttccttcaacttagttggattattcggaacaaaaacaaaatcttctgattttaatacagcgaagcttgttcctaacattgctttgaatcgagctgtggttgagctttatacagacactacctttgcttggagcgtaggtgctcgtgcagctctctgggaatgtgggtgtgcaacgttaggagcttctttccaatatgctcaatctaaacctaaagtagaagagttaaatgttctttgtaatgcatccgaatttactattaataagccgaaaggatatgttggggcggaatttccacttgatattaccgcaggaacagaagctgcgacagggactaaggatgcctctattgactaccatgagtggcaagcaagtttagccctttcttacagactaaatatgttcactccttacattggagttaaatggtctagagtaagttttgatgccgacacgatccgtatcgctcagcctaaattggctgaagcaatcttggatgtcactactctaaacccgaccatcgctggtaaaggaactgtggtcgcttccggaagcgataacgacctggctgatacaatgcaaatcgtttccttgcagttgaacaagatgaaatctagaaaatcttgcggtattgcagtaggaacgactattgtagatgcagacaaatacgcagttacagttgagactcgcttgatcgatgagagagcagctcacgtaaatgcacaattccgcttctaa

SEQ ID NO: 52 - protein, 397 aa, Chlamydia trachomatis
  misc_feature: Major Outer Membrane Protein (Momp) or Ct-681 from serovar H
MKKLLKSVLVFAALSSASSLQALPVGNPAEPSLMIDGILWEGFGGDPCDPCATWCDAISMRVGYYGDFVFDRVLKTDVNKEFQMGAAPTTNDAADLQNDPKTNVARPNPAYGKHMQDAEMFTNAAYMALNIWDRFDVFCTLGATTGYLKGNSASFNLVGLFGTKTKSSDFNTAKLVPNIALNRAVVELYTDTTFAWSVGARAALWECGCATLGASFQYAQSKPKVEELNVLCNASEFTINKPKGYVGAEFPLDITAGTEAATGTKDASIDYHEWQASLALSYRLNMFTPYIGVKWSRVSFDADTIRIAQPKLAEAILDVTTLNPTIAGKGTVVASGSDNDLADTMQIVSLQLNKMKSRKSCGIAVGTTIVDADKYAVTVETRLIDERAAHVNAQFRF

SEQ ID NO: 53 - DNA, 1182 bp, Chlamydia trachomatis
  misc_feature: Major Outer Membrane Protein (Momp) or Ct-681 from serovar E
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcaccacttggtgtgacgctatcagcatgcgtatgggttactatggtgactttgttttcgaccgtgttttgaaaacagatgtgaataaagaattccaaatgggtgacaagcctacaagtactacaggcaatgctacagctccaaccactcttacagcaagagagaatcctgcttacggccgacatatgcaggatgctgagatgtttacaaatgccgcttgcatggcattgaatatttgggatcgctttgatgtattctgtacactaggagcctctagcggataccttaaaggaaactctgcttctttcaatttagttggattgtttggagataatgaaaatcaaagcacggtcaaaacgaattctgtaccaaatatgagcttagatcaatctgttgttgaactttacacagatactgccttctcttggagcgtgggcgctcgagcagctttgtgggagtgcggatgtgcgactttaggggcttctttccaatacgctcaatctaaacctaaagtcgaagaattaaacgttctctgtaacgcagctgagtttactatcaataagcctaaaggatatgtagggcaagaattccctcttgcactcatagcaggaactgatgcagcgacgggcactaaagatgcctctattgattaccatgagtggcaagcaagtttagctctctcttacagattgaatatgttcactccctacattggagttaaatggtctcgagcaagttttgatgccgatacgattcgtatagcccagccaaaatcagctacagctatctttgatactaccacgcttaacccaactattgctggagctggcgatgtgaaagctagcgcagagggtcagctcggagataccatgcaaatcgtctccttgcaattgaacaagatgaaatctagaaaatcttgcggtattgcagtaggaacgactattgtagatgcagacaaatacgcagttacagttgagactcgcttgatcgatgagagagctgctcacgtaaatgcacaattccgcttctaa

SEQ ID NO: 54 - protein, 393 aa, Chlamydia trachomatis
  misc_feature: Major Outer Membrane Protein (Momp) or Ct-681 from serovar E
MKKLLKSVLVFAALSSASSLQALPVGNPAEPSLMIDGILWEGFGGDPCDPCTTWCDAISMRMGYYGDFVFDRVLKTDVNKEFQMGDKPTSTTGNATAPTTLTARENPAYGRHMQDAEMFTNAACMALNIWDRFDVFCTLGASSGYLKGNSASFNLVGLFGDNENQSTVKTNSVPNMSLDQSVVELYTDTAFSWSVGARAALWECGCATLGASFQYAQSKPKVEELNVLCNAAEFTINKPKGYVGQEFPLALIAGTDAATGTKDASIDYHEWQASLALSYRLNMFTPYIGVKWSRASFDADTIRIAQPKSATAIFDTTTLNPTIAGAGDVKASAEGQLGDTMQIVSLQLNKMKSRKSCGIAVGTTIVDADKYAVTVETRLIDERAAHVNAQFRF

SEQ ID NO: 55 - DNA, 1179 bp, Chlamydia trachomatis
  misc_feature: Major Outer Membrane Protein (Momp) or Ct-681 from serovar D
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcgccacttggtgtgacgctatcagcatgcgtgttggttactacggagactttgttttcgaccgtgttttgaaaactgatgtgaataaagaatttcagatgggtgccaagcctacaactgatacaggcaatagtgcagctccatccactcttacagcaagagagaatcctgcttacggccgacatatgcaggatgctgagatgtttacaaatgccgcttgcatggcattgaatatttgggatcgttttgatgtattctgtacattaggagccaccagtggatatcttaaaggaaactctgcttctttcaatttagttggattgtttggagataatgaaaatcaaaaaacggtcaaagcggagtctgtaccaaatatgagctttgatcaatctgttgttgagttgtatacagatactacttttgcgtggagcgtcggcgctcgcgcagctttgtgggaatgtggatgtgcaactttaggagcttcattccaatatgctcaatctaaacctaaagtagaagaattaaacgttctctgcaatgcagcagagtttactattaataaacctaaagggtatgtaggtaaggagtttcctcttgatcttacagcaggaacagatgctgcgacaggaactaaggatgcctctattgattaccatgaatggcaagcaagtttagctctctcttacagactgaatatgttcactccctacattggagttaaatggtctcgagcaagctttgatgccgatacgattcgtatagcccagccaaaatcagctacagctatttttgatactaccacgcttaacccaactattgctggagctggcgatgtgaaaactggcgcagagggtcagctcggagacacaatgcaaatcgtttccttgcaattgaacaagatgaaatctagaaaatcttgcggtattgcagtaggaacaactattgtggatgcagacaaatacgcagttacagttgagactcgcttgatcgatgagagagcagctcacgtaaatgcacaattccgcttc

SEQ ID NO: 56 - protein, 393 aa, Chlamydia trachomatis
  misc_feature: Major Outer Membrane Protein (Momp) or Ct-681 from serovar D
MKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 57 - DNA, 1944 bp, Chlamydia trachomatis
  misc_feature: Ct-622 from serovar D
atggaatcaggaccagaatcagtttcttctaatcagagctcgatgaatccaattattaatgggcaaatcgcttctaattcggagaccaaagagtccacgaaggagtcagaagcgagtccttcagcatcgtcctctgtaagcagctggagttttttatcctcagcaaagcatgcattaatctctcttcgtgatgccatcttgaataaaaattctagtccaacagactctctctctcaattagaggcctctacttctacctctacggttacacgtgtagctgcgcgagattataatgaggctaaatcgaattttgatacggcgaaaagtggattagagaacgctacgacacttgctgaatacgagacgaaaatggctgatttaatggcagctctccaagatatggagcgtttggctaaacagaaggctgaagttacaagaattaaagaagctcttcaagagaaacaagaggttattgataagctcaatcagttagttaaacttgaaaaacagaatcagactttaaaggaaactttaacaaccacagactctgcagatcagattccagcgattaatagtcagttagagatcaacaaaaattctgcagatcaaattatcaaagatctggaaggacaaaacataagttatgaagctgttctcactaacgcaggagaggttatcaaagcttcttctgaagcgggaattaagttaggacaagctttgcagtctattgtggatgctggggatcaaagccaggctgcagttcttcaagcacagcaaaataatagcccagataatatcgcagccacgaagaaattaattgatgctgctgaaacgaaggtaaacgagttaaaacaagagcatacagggctaacggactcgcctttagtgaaaaaagctgaggagcagattagtcaagcacaaaaagatattcaagagatcaaacctagtggttcggatattcctatcgttggtccgagtgggtcagctgcttccgcaggaagtgcggtaggagcgttgaaatcctctaacaattcaggaagaatttccttgttgcttgatgatgtagacaatgaaatggcagcgattgcaatgcaaggttttcgatctatgatcgaacaatttaatgtaaacaatcctgcaacagctaaagagctacaagctatggaggctcagctgactgcgatgtcagatcaactggttggtgcggatggcgagctcccagccgaaatacaagcaatcaaagatgctcttgcgcaagctttgaaacaaccatcaacagatggtttagctacagctatgggacaagtggcttttgcagctgccaaggttggaggaggctccgcaggaacagctggcactgtccagatgaatgtaaaacagctttacaagacagcgttttcttcgacttcttccagctcttatgcagcagcactttccgatggatattctgcttacaaaacactgaactctttatattccgaaagcagaagcggcgtgcagtcagctattagtcaaactgcaaatcccgcgctttccagaagcgtttctcgttctggcatagaaagtcaaggacgcagtgcagatgctagccaaagagcagcagaaactattgtcagagatagccaaacgttaggtgatgtatatagccgcttacaggttctggattctttgatgtctacgattgtgagcaatccgcaagtaaatcaagaagagattatgcagaagctcacggcatctattagcaaagctccacaatttgggtatcctgctgttcagaattctgcggatagcttgcagaagtttgctgcgcaattggaaagagagtttgttgatggggaacgtagtctcgcagaatctcgagagaatgcgtttagaaaacagcccgctttcattcaacaggtgttggtaaacattgcttctctattctctggttatctttcttaa

SEQ ID NO: 58 - protein, 647 aa, Chlamydia trachomatis
  misc_feature: Ct-622 from serovar D
MESGPESVSSNQSSMNPIINGQIASNSETKESTKESEASPSASSSVSSWSFLSSAKHALISLRDAILNKNSSPTDSLSQLEASTSTSTVTRVAARDYNEAKSNFDTAKSGLENATTLAEYETKMADLMAALQDMERLAKQKAEVTRIKEALQEKQEVIDKLNQLVKLEKQNQTLKETLTTTDSADQIPAINSQLEINKNSADQIIKDLEGQNISYEAVLTNAGEVIKASSEAGIKLGQALQSIVDAGDQSQAAVLQAQQNNSPDNIAATKKLIDAAETKVNELKQEHTGLTDSPLVKKAEEQISQAQKDIQEIKPSGSDIPIVGPSGSAASAGSAVGALKSSNNSGRISLLLDDVDNEMAAIAMQGFRSMIEQFNVNNPATAKELQAMEAQLTAMSDQLVGADGELPAEIQAIKDALAQALKQPSTDGLATAMGQVAFAAAKVGGGSAGTAGTVQMNVKQLYKTAFSSTSSSSYAAALSDGYSAYKTLNSLYSESRSGVQSAISQTANPALSRSVSRSGIESQGRSADASQRAAETIVRDSQTLGDVYSRLQVLDSLMSTIVSNPQVNQEEIMQKLTASISKAPQFGYPAVQNSADSLQKFAAQLEREFVDGERSLAESRENAFRKQPAFIQQVLVNIASLFSGYLS

SEQ ID NO: 59-62 - skipped (4 entries)

SEQ ID NO: 63 - DNA, 261 bp, Chlamydia trachomatis
  misc_feature: Ct-460, also known as Swib, from serovar D
atgagtcaaaataagaactctgctttcatgcagcctgtgaacgtatccgctgatttagctgccatcgttggtgcaggacctatgcctcgcacagagatcattaagaaaatgtgggattacattaagaagaatggccttcaagatcctacaaacaaacgtaatatcaatcccgatgataaattggctaaagtttttggaactgaaaaacctatcgatatgttccaaatgacaaaaatggtttctcaacacatcattaaataa

SEQ ID NO: 64 - protein, 86 aa, Chlamydia trachomatis
  misc_feature: Ct-460, also known as Swib, from serovar D
MSQNKNSAFMQPVNVSADLAAIVGAGPMPRTEIIKKMWDYIKKNGLQDPTNKRNINPDDKLAKVFGTEKPIDMFQMTKMVSQHIIK

SEQ ID NO: 65-70 - skipped (6 entries)

SEQ ID NO: 71 - DNA, 1266 bp, Chlamydia trachomatis
  misc_feature: Ct-089 from serovar D
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaaggagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgacgaaattctcgatgcgctcacaagtaaattttctgatcccacaataaaggatctagctcttgattatctaattcaaacagctccctctgatgggaaacttaagtccactctcattcaggcaaagcatcaactgatgagccagaatcctcaggcgattgttggaggacgcaatgttctgttagcttcagaaacctttgcttccagagcaaatacatctccttcatcgcttcgctccttatatttccaagtaacctcatccccctctaattgcgctaatttacatcaaatgcttgcttcttacttgccatcagagaaaaccgctgttatggagtttctagtaaatggcatggtagcagatttaaaatcggagggcccttccattcctcctgcaaaattgcaagtatatatgacggaactaagcaatctccaagccttacactctgtaaatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 72 - protein, 421 aa, Chlamydia trachomatis
  misc_feature: Ct-089 from serovar D
MTASGGAGGLGSTQTVDVARAQAAAATQDAQEVIGSQEASEASMLKGCEDLINPAAATRIKKKGEKFESLEARRKPTADKAEKKSESTEEKGDTPLEDRFTEDLSEVSGEDFRGLKNSFDDDSSPDEILDALTSKFSDPTIKDLALDYLIQTAPSDGKLKSTLIQAKHQLMSQNPQAIVGGRNVLLASETFASRANTSPSSLRSLYFQVTSSPSNCANLHQMLASYLPSEKTAVMEFLVNGMVADLKSEGPSIPPAKLQVYMTELSNLQALHSVNSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 73-78 - skipped (6 entries)

SEQ ID NO: 79 - DNA, 1266 bp, Chlamydia trachomatis
  misc_feature: Ct-089 from serovar A
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgaagaaattctcgatgcgctcacaagtaaattttctgatcccacaataaaggatctagctcttgattatctaattcaaacagctccctctgataggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatttccaagtaacctcatccccctctaattgtgataatttacgtcaaatgcttgcttcttactcgccatcagagaaaaccgctgttatggagtttctagtaaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 80 - protein, 421 aa, Chlamydia trachomatis
  misc_feature: Ct-089 from serovar A
MTASGGAGGLGSTQTVDVARAQAAAATQDAQEVIGSQEASEASMLKGCEDLINPAAATRIKKKEEKFESLEARRKPTADKAEKKSESTEEKGDTPLEDRFTEDLSEVSGEDFRGLKNSFDDDSSPEEILDALTSKFSDPTIKDLALDYLIQTAPSDRKLKSALIQAKHQLMSQNPQAIVGGRNVLLASETFASRANTSPSSLRSLYFQVTSSPSNCDNLRQMLASYSPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 81 - DNA, 1266 bp, Chlamydia trachomatis
  misc_feature: Ct-089 from serovar B
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgaagaaattctcgatgcgctcacaagtaaattttctgatcccacaataaaggatctagctcttgattatctaattcaaacagctccctctgataggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatctccaagtaacctcatccccctctaattgtgataatttacgtcaaatgcttgcttcttactcgccatcagagaaaaccgctgttatggagtttctagtaaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 82 - protein, 421 aa, Chlamydia trachomatis
  misc_feature: Ct-089 from serovar B
MTASGGAGGLGSTQTVDVARAQAAAATQDAQEVIGSQEASEASMLKGCEDLINPAAATRIKKKEEKFESLEARRKPTADKAEKKSESTEEKGDTPLEDRFTEDLSEVSGEDFRGLKNSFDDDSSPEEILDALTSKFSDPTIKDLALDYLIQTAPSDRKLKSALIQAKHQLMSQNPQAIVGGRNVLLASETFASRANTSPSSLRSLYLQVTSSPSNCDNLRQMLASYSPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 83 - DNA, 1266 bp, Chlamydia trachomatis; identical to SEQ ID NO: 71
  misc_feature: Ct-089 from serovar G

SEQ ID NO: 84 - protein, 421 aa, Chlamydia trachomatis; identical to SEQ ID NO: 72
  misc_feature: Ct-089 from serovar G

SEQ ID NO: 85 - DNA, 1266 bp, Chlamydia trachomatis
  misc_feature: Ct-089 from serovar H
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgacgaaattctcgatgcgctcacaagtaaattttctgatcccacaataaaggatctagctcttgattatctaattcaaacagctccctctgatgggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatttccaagtaacctcatccccctctaattgcgctaatttacatcaaatgcttgcttcttacttgccatcagagaaaaccgctgttatggagtttctagtaaatggcatggtagcagatttaaaatcggagggcccttccattcctcctgcaaaattgcaagtatatatgacggaactaagcaatctccaagccttacactctgtaaatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 86 - protein, 421 aa, Chlamydia trachomatis
  misc_feature: Ct-089 from serovar H
MTASGGAGGLGSTQTVDVARAQAAAATQDAQEVIGSQEASEASMLKGCEDLINPAAATRIKKKEEKFESLEARRKPTADKAEKKSESTEEKGDTPLEDRFTEDLSEVSGEDFRGLKNSFDDDSSPDEILDALTSKFSDPTIKDLALDYLIQTAPSDGKLKSALIQAKHQLMSQNPQAIVGGRNVLLASETFASRANTSPSSLRSLYFQVTSSPSNCANLHQMLASYLPSEKTAVMEFLVNGMVADLKSEGPSIPPAKLQVYMTELSNLQALHSVNSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 87 - DNA, 1266 bp, Chlamydia trachomatis; identical to SEQ ID NO: 71
  misc_feature: Ct-089 from serovar I

SEQ ID NO: 88 - protein, 421 aa, Chlamydia trachomatis; identical to SEQ ID NO: 72
  misc_feature: Ct-089 from serovar I

SEQ ID NO: 89 - DNA, 1266 bp, Chlamydia trachomatis
  misc_feature: Ct-089 from serovar J
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaagaatgtgcggatctcataaatcctgcagctgcaacccgaatcaaaaaaaaaaaagagaagtttgaatcattagaagctcgtcgcaaaccaacagcggataaagcagaaaagaaatccgagagcacagaggaaaaaggcgatactcctcttgaagatcgtttcacagaagatctttccgaagtctctggagaagattttcgaggattgaaaaattcgttcgatgatgattcttcttctgacgaaattctcgatgcgctcacaagtaaattttctgatcccacaataaaggatctagctcttgattatctaattcaaatagctccctctgatgggaaacttaagtccactctcattcaggcaaagcatcaactgatgagccagaatcctcaggcgattgttggaggacgcaatgttctgttagcttcagaaacctttgcttccagagcaaatacatctccttcatcgcttcgctccttatatctccaagtaacctcatccccctctaattgcgctaatttacatcaaatgcttgcttcttactcgccatcagagaaaaccgctgttatggagtttctagtgaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 90 - protein, 421 aa, Chlamydia trachomatis
  misc_feature: Ct-089 from serovar J
MTASGGAGGLGSTQTVDVARAQAAAATQDAQEVIGSQEASEASMLKECADLINPAAATRIKKKKEKFESLEARRKPTADKAEKKSESTEEKGDTPLEDRFTEDLSEVSGEDFRGLKNSFDDDSSSDEILDALTSKFSDPTIKDLALDYLIQIAPSDGKLKSTLIQAKHQLMSQNPQAIVGGRNVLLASETFASRANTSPSSLRSLYLQVTSSPSNCANLHQMLASYSPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 91 - DNA, 1266 bp, Chlamydia trachomatis; identical to SEQ ID NO: 71
  misc_feature: Ct-089 from serovar K

SEQ ID NO: 92 - protein, 421 aa, Chlamydia trachomatis; identical to SEQ ID NO: 72
  misc_feature: Ct-089 from serovar K

SEQ ID NO: 93 - DNA, 1266 bp, Chlamydia trachomatis
  misc_feature: Ct-089 from serovar L2
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaagaatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctctggagaagattttcgaggattgaaaaattcgttcgatgatgattcttcttctgacgaaattctcgatgcgctcacaagtaaattttctgatcccacaataaaggatctagctcttgattatctaattcaaatagctccctctgatgggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatttccaagtaacctcatccccctctaattgcgctaatttacatcaaatgcttgcttcttactcgccatcagagaaaaccgctgttatggagtttctagtgaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccggatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 94 - protein, 421 aa, Chlamydia trachomatis
  misc_feature: Ct-089 from serovar L2
MTASGGAGGLGSTQTVDVARAQAAAATQDAQEVIGSQEASEASMLKECEDLINPAAATRIKKKEEKFESLEARRKPTADKAEKKSESTEEKGDTPLEDRFTEDLSEVSGEDFRGLKNSFDDDSSSDEILDALTSKFSDPTIKDLALDYLIQIAPSDGKLKSALIQAKHQLMSQNPQAIVGGRNVLLASETFASRANTSPSSLRSLYFQVTSSPSNCANLHQMLASYSPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 95 - DNA, 1749 bp, Chlamydia trachomatis
  misc_feature: Ct-858 from serovar A
atggtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccttaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggartctcgccttgctctgggagacaacatggaaggatatactgtggatctacaggttgccgagtatttaaaaagctttggacgtcaagtattgaattgttggagtaaaggggatattgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttag

SEQ ID NO: 96 - protein, 580 aa, Chlamydia trachomatis
  misc_feature: Ct-858 from serovar A
MVQGESLVCKNALQDLSFLEHLLQVKYAPKTWKEQYLGWDLVQSSVSAQQKLRTQENPSTSFCQQVLADFIGQDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFLKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 97 - DNA, 1749 bp, Chlamydia trachomatis
  misc_feature: Ct-858 from serovar B
atggtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggaatctcgccttgctctgggagacaacatggaaggatatactgtggatctacaggttgccgagtatttaaaaagctttggacgtcaagtattgaattgttggagtaaaggggatatcgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttag

SEQ ID NO: 98 - protein, 580 aa, Chlamydia trachomatis
  misc_feature: Ct-858 from serovar B
MVQGESLVCKNALQDLSFLEHLLQVKYAPKTWKEQYLGWDLVQSSVSAQQKLRTQENPSTSFCQQVLADFIGQDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 99 - DNA, 1749 bp, Chlamydia trachomatis
  misc_feature: Ct-858 from serovar G
atggtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttag

SEQ ID NO: 100 - protein, 580 aa, Chlamydia trachomatis; identical to SEQ ID NO: 98
  misc_feature: Ct-858 from serovar G

SEQ ID NO: 101 - DNA, 1749 bp, Chlamydia trachomatis; identical to SEQ ID NO: 99
  misc_feature: Ct-858 from serovar H

SEQ ID NO: 102 - protein, 580 aa, Chlamydia trachomatis; identical to SEQ ID NO: 98
  misc_feature: Ct-858 from serovar H

SEQ ID NO: 103 - DNA, 1749 bp, Chlamydia trachomatis; identical to SEQ ID NO: 99
  misc_feature: Ct-858 from serovar I

SEQ ID NO: 104 - protein, 580 aa, Chlamydia trachomatis; identical to SEQ ID NO: 98
  misc_feature: Ct-858 from serovar I

SEQ ID NO: 105 - DNA, 1749 bp, Chlamydia trachomatis
  misc_feature: Ct-858 from serovar J
atggtacgaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtgctcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacacctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttag

SEQ ID NO: 106 - protein, 580 aa, Chlamydia trachomatis
  misc_feature: Ct-858 from serovar J
MVRGESLVCKNALQDLSFLEHLLQVKYAPKTWKEQYLGWDLVQSSVSAQQKLRTQENPSTSFCQQVLADFIGQDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFLKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 107 - DNA, 1749 bp, Chlamydia trachomatis; identical to SEQ ID NO: 99
  misc_feature: Ct-858 from serovar K

SEQ ID NO: 108 - protein, 580 aa, Chlamydia trachomatis; identical to SEQ ID NO: 96
  misc_feature: Ct-858 from serovar K

SEQ ID NO: 109 - DNA, 1749 bp, Chlamydia trachomatis; identical to SEQ ID NO: 105
  misc_feature: Ct-858 from serovar L2

SEQ ID NO: 110 - protein, 580 aa, Chlamydia trachomatis; identical to SEQ ID NO: 106
  misc_feature: Ct-858 from serovar L2

SEQ ID NO: 111 - DNA, 1770 bp, Chlamydia trachomatis
  misc_feature: Ct-875 from serovar A
atgagcatcaggggagtaggaggcaacgggaatagtcgaatcccttctcataatggggatggatcgaatcgcagaagtcaaaatacgaagaataaagttgaagatcgagttcgttctctatattcatctcgtagtaacgaaaatagagaatctccttatgcagtagtagacgtcagctctatgatcgagagcaccccaacgagtggagagacgacaagagcttcgcgtggagtattcagtcgtttccaaagaggtttaggacgagtagctgacaaagtaagacgagctgttcagcgtgcgtggagttcagtctctataagaagatcgtctgcaacaagagccacagaatccagatcaagtagtcgtactgctcgtggtgcaagttctgggtataaggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaaggagttggctggccctgacggggtagcggccgcccgggaaattgctaaaagatgggagaaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagcacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagagacctgatgaagcgttggaatagagaagtcgatagggaataa

SEQ ID NO: 112 - protein, 581 aa, Chlamydia trachomatis
  misc_feature: Ct-875 from serovar A
MSIRGVGGNGNSRIPSHNGDGSNRRSQNTKNKVEDRVRSKSRSNENRESPYAVVDVSSMIESTPTSGETTRASRGVFSRFQRGLGRVADKVRRAVQRAWSSVSIRRSSATRATESRSSSRTRASSGYKEYSPSARLRLMFTDFWRTRVLRQTSPMAGVFGNLDVNEARLMAAYTSECADHLEAKELAGPDGVAAAREIAKRWEKRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTYAPRDDLSPEGASLAETLARFADDMGIERGADGTYDIPLVDDWRRGVPSIEGEGSDSIYEIMMPIYEVMNMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMKRWNREVDRE

SEQ ID NO: 113 - DNA, 1770 bp, Chlamydia trachomatis
  misc_feature: Ct-875 from serovar B
atgagcatcaggggagtaggaggcaacgggaatagtcgaatcccttctcataatggggatggatcgaatcgcagaagtcaaaatacgaagaataaagttgaagatcgagttcgttctctatattcatctcgtagtaacgaaaatagagaatctccttatgcagtagtagacgtcagctctatgatcgagagcaccccaacgagtggagagacgacaagagcttcgcgtggagtattcagtcgtttccaaagaggtttaggacgagtagctgacaaagtaagacgagctgttcagcgtgcgtggagttcagtctctataagaagatcgtctgcaacaagagccgcagaatccagatcaagtagtcgtactgctcgtggtgcaagttctgggtatagggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaaggagttggctggccctgacggggtagcggccgcccgggaaattgctaaaagatgggagaaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagcacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtacaattatgtagtgacacagccgcaagagcgtattcccaatagtcagcaggtggaagagattctgcgtgatatgcttaccaacgggtcacagacatttagagacctgatgaagcgttggaatagagaagtcgatagggaataa

SEQ ID NO: 114 - protein, 581 aa, Chlamydia trachomatis
  misc_feature: Ct-875 from serovar B
MSIRGVGGNGNSRIPSHNGDGSNRRSQNTKNKVEDRVRSKSRSNENRESPYAVVDVSSMIESTPTSGETTRASRGVFSRFQRGLGRVADKVRRAVQRAWSSVSIRRSSATRAAESRSSSRTRASSGYREYSPSARLRLMFTDFWRTRVLRQTSPMAGVFGNLDVNEARLMAAYTSECADHLEAKELAGPDGVAAAREIAKRWEKRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTYAPRDDLSPEGASLAETLARFADDMGIERGADGTYDIPLVDDWRRGVPSIEGEGSDSIYEIMMPIYEVMNMDLETRRSFAVQQGHYQDPRASDYDLPRASDYDLPRSPYPTPPLPPRYQLQNMDVEAGFREAVYASFVAGMYNYVVTQPQERIPNSQQVEEILRDMLTNGSQTFRDLMKRWNREVDRE

SEQ ID NO: 115 - DNA, 1776 bp, Chlamydia trachomatis; identical to SEQ ID NO: 21
  misc_feature: Ct-875 from serovar G

SEQ ID NO: 116 - protein, 583 aa, Chlamydia trachomatis
  misc_feature: Ct-875 from serovar G
MSIRGVGGNGNSRIPSHNGDGSNRRSQNTKGNNKVEDRVCSKSRSNENRESPYAVVDVSSMIESTPTSGETTRASRGVFSRFQRGLVRVADKVRRAVQCAWSSVSTRRSSATRAAESGSSSRTRASSGYREYSPSARLRLMFTDFWRTRVLRQTSPMAGVFGNLDVNEARLMAAYTSECADHLEANKLAGPDGVAAAREIAKRWEQRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTYAPRDDLSPEGASLAETLARFADDMGIERGADGTYDIPLVDDWRRGVPSIEGEGSDSIYEIMMPIYEVMDMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMRRWNREVDRE

SEQ ID NO: 117 - DNA, 1776 bp, Chlamydia trachomatis
  misc_feature: Ct-875 from serovar H
atgagcatcaggggagtaggaggcaacgggaatagtcgaatcccttctcataatggggatggatcgaatcgcagaagtcaaaatacgaagggtaataataaagttgaagatcgagtttgttctctatattcatctcgtagtaacgaaaatagagaatctccttatgcagtagtagacgtcagctctatgatcgagagcaccccaacgagtggagagacgacaagagcttcgcgtggagtgttcagtcgtttccaaagaggtttagtacgagtagctgacaaagtaagacgagctgttcagtgtgcgtggagttcagtctctacaagaagatcgtctgcaacaagagccgcagaatccggatcaagtagtcgtactgctcgtggtgcaagttctgggtatagggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaacaagttggctggccctgacggggtagcggccgcccgggaaattgctaaaagatgggagcaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagcacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatggatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtataattatgtagtgacacagccgcaagagcgtattcccaatagtcagcaggtggaagggattctgcgtgatatgcttaccaacgggtcacagacatttagagacctgatgaagcgttggaatagagaagtcgatagggaataa

SEQ ID NO: 118 - protein, 583 aa, Chlamydia trachomatis
  misc_feature: Ct-875 from serovar H
MSIRGVGGNGNSRIPSHNGDGSNRRSQNTKGNNKVEDRVCSKSRSNENRESPYAVVDVSSMIESTPTSGETTRASRGVFSRFQRGLVRVADKVRRAVQCAWSSVSTRRSSATRAAESGSSSRTRASSGYREYSPSARLRLMFTDFWRTRVLRQTSPMAGVFGNLDVNEARLMAAYTSECADHLEANKLAGPDGVAAAREIAKRWEQRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTYAPRDDLSPEGASLAETLARFADDMGIERGADGTYDIPLVDDWRRGVPSIEGEGSDSIYEIMMPIYEVMDMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMKRWNREVDRE

SEQ ID NO: 119 - DNA, 1776 bp, Chlamydia trachomatis; identical to SEQ ID NO: 21
  misc_feature: Ct-875 from serovar I

SEQ ID NO: 120 - protein, 583 aa, Chlamydia trachomatis; identical to SEQ ID NO: 116
  misc_feature: Ct-875 from serovar I

SEQ ID NO: 121 - DNA, 1773 bp, Chlamydia trachomatis
  misc_feature: Ct-875 from serovar J
atgagcatcaggggagtaggaggcaacgggaatagtcgaatcccttctcataatggggatggatcgaatcgcagaagtcaaaatacgaagggtaataataaagttgaagatcgagttcattctctatattcatctcttagtaacgaaaatagagaatctccttatccagtagtagacgtcagctctatgatcgagagcaccccaacgagtggagagacgccaagagcttcgcgtggagtgttcagtcgtttccaaagaggtttaggacgagtagctgacaaagtaagacgagctgttcagtgtgcgtggggttcagtctctacaagaagatcgtctgcaacaagagccgtagaatccggatcaagtagtcgtactgctcgtggtgcaagttctgggagggagtattctccttcagcagctagagggctgcgtcttatgttcacagatttctggagaactcgggttttacgccagacctctcctatggatgtagtttttgggaatcttgatgtgaacgaggctcgtttgatggctgcttacacaagtgagtgcgcggattatttagaagcgcacgatttggctggccctgacggggtagcggccgcccgggaaattgctcaaagatgggagaaaagagttagagatctacaagataaaggtgctgcacaaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatttctcagacgttgtattatccagagagattcaaacagggcttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatgggaggcgatgagacgcaaggaccttctagagcacaacaagcttttcagagctttgtaaatgaatgtaatagcatcgagttctcatttgggagctttggagagcatgtgcgagttctctgcgctagagtatcacgaggattagttgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcaaggtgctgatggaacctacgatattccttgggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgccttctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagcgacctgatgaagcgttgggatagagaagtcgatagggaataa

SEQ ID NO: 122 - protein, 582 aa, Chlamydia trachomatis
  misc_feature: Ct-875 from serovar J
MSIRGVGGNGNSRIPSHNGDGSNRRSQNTKGNNKVEDRVHSKSLSNENRESPYPVVDVSSMIESTPTSGETPRASRGVFSRFQRGLGRVADKVRRAVQCAWGSVSTRRSSATRAVESGSSSRTRASSGREYSPSARLRLMFTDFWRTRVLRQTSPMDVVFGNLDVNEARLMAAYTSECADYLEAHDLAGPDGVAAAREIAQRWEKRVRDLQDKGAAQKLLNDPLGRRTPNYQSKNPGEYTVGNSMFYDGPQVANLQNVDTGFWLDMSNFSDVVLSREIQTGLRARATLEESMPMLENLEERFRRLQETCDAARTEIEESGWTRESASRMGGDETQGPSRAQQAFQSFVNECNSIEFSFGSFGEHVRVLCARVSRGLVAAGEAIRRCFSCCKGSTYAPRDDLSPEGASLAETLARFADDMGIEQGADGTYDIPWVDDWRRGVPSIEGEGSDSIYEIMMPIYEVMNMDLETRRSFAVQQGHYQDPRASDYDLPRASDYDLPRSPYPTPPLPSRYQLQNMDVEAGFREAVYASFVAGMYNYVVTQPQERIPNSQQVEGILRDMLTNGSQTFSDLMKRWDREVDRE

SEQ ID NO: 123 - DNA, 1776 bp, Chlamydia trachomatis; identical to SEQ ID NO: 21
  misc_feature: Ct-875 from serovar K

SEQ ID NO: 124 - protein, 583 aa, Chlamydia trachomatis; identical to SEQ ID NO: 116
  misc_feature: Ct-875 from serovar K

SEQ ID NO: 125 - DNA, 1773 bp, Chlamydia trachomatis
  misc_feature: Ct-875 from serovar L2
atgagcatcaggggagtaggaggcaacgggaatagtcgaatcccttctcataatggggatggatcgaatcgcagaagtcaaaatacgaagggtaataataaagttgaagatcgagttcattctctatattcatctcttagtaacgaaaatagagaatctccttatccagtagtagacgtcagctctatgatcgagagcaccccaacgagtggagagacgccaagagcttcgcgtggagtgttcagtcgtttccaaagaggtttaggacgagtagctgacaaagtaagacgagctgttcagtgtgcgtggggttcagtctctacaagaagatcgtctgcaacaagagccgtagaatccggatcaagtagtcgtactgctcgtggtgcaagttctgggagggagtattctccttcagcagctagagggctgcgtcttatgttcacagatttctggagaactcgggttttacgccagacctctcctatggatgtagtttttgggaatcttgatgtgaacgaggctcgtttgatggctgcttacacaagtgagtgcgcggattatttagaagcgcacgatttggctggccctgacggggtagcggccgcccgggaaattgctcaaagatgggataaaagagttagagatctacaagataaaggtgctgcacaaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatttctcagacgttgtattatccagagagattcaaacagggcttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatgggaggcgatgagacgcaaggaccttctagagcacaacaagcttttcagagctttgtaaatgaatgtaatagcatcgagttctcatttgggagctttggagagcatgtgcgagttctctgcgctagagtatcacgaggattagttgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcaaggtgctgatggaacctacgatattccttgggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgccttctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagcaacctgatgcagcgttgggatagagaagtcgatagggaataa

SEQ ID NO: 126 - protein, 582 aa, Chlamydia trachomatis
  misc_feature: Ct-875 from serovar L2
MSIRGVGGNGNSRIPSHNGDGSNRRSQNTKGNNKVEDRVHSKSLSNENRESPYPVVDVSSMIESTPTSGETPRASRGVFSRFQRGLGRVADKVRRAVQCAWGSVSTRRSSATRAVESGSSSRTRASSGREYSPSARLRLMFTDFWRTRVLRQTSPMDVVFGNLDVNEARLMAAYTSECADYLEAHDLAGPDGVAAAREIAQRWDKRVRDLQDKGAAQKLLNDPLGRRTPNYQSKNPGEYTVGNSMFYDGPQVANLQNVDTGFWLDMSNFSDVVLSREIQTGLRARATLEESMPMLENLEERFRRLQETCDAARTEIEESGWTRESASRMGGDETQGPSRAQQAFQSFVNECNSIEFSFGSFGEHVRVLCARVSRGLVAAGEAIRRCFSCCKGSTYAPRDDLSPEGASLAETLARFADDMGIEQGADGTYDIPWVDDWRRGVPSIEGEGSDSIYEIMMPIYEVMNMDLETRRSFAVQQGHYQDPRASDYDLPRASDYDLPRSPYPTPPLPSRYQLQNMDVEAGFREAVYASFVAGMYNYVVTQPQERIPNSQQVEGILRDMLTNGSQTFSNLMQRWDREVDRE